SEQUENCE LISTING (119 sequences)

SEQ ID NO: 1 - protein, 5 aa, synthetic construct
  source 1..5: note = HT12-GL HCDR1
IYAMT

SEQ ID NO: 2 - protein, 17 aa, synthetic construct
  source 1..17: note = HT12-GL HCDR2
GISAGGDSTYYADSVKG

SEQ ID NO: 3 - protein, 10 aa, synthetic construct
  source 1..10: note = HT12-GL HCDR3
DRGYYYGMDV

SEQ ID NO: 4 - protein, 16 aa, synthetic construct
  source 1..16: note = HT12-GL LCDR1
RSRQSLLRSNGYNYLD

SEQ ID NO: 5 - protein, 7 aa, synthetic construct
  source 1..7: note = HT12-GL LCDR2
LGSNRAS

SEQ ID NO: 6 - protein, 9 aa, synthetic construct
  source 1..9: note = HT12-GL LCDR3
MQALQTPYT

SEQ ID NO: 7 - protein, 119 aa, synthetic construct
  source 1..119: note = HT12-GL VH
EVQLLESGGGLVQPGGSLRLSCAASGFTFNIYAMTWVRQAPGKGLEWVSGISAGGDSTYYADSVKGRFTISRDNSKNTLYLQMNSLRAEDTAVYYCAKDRGYYYGMDVWGQGTTVTVSS

SEQ ID NO: 8 - protein, 112 aa, synthetic construct
  source 1..112: note = HT12-GL VL
DIVMTQSPLSLPVTPGEPASISCRSRQSLLRSNGYNYLDWYLQKPGQSPQLLIYLGSNRASGVPDRFSGSGSGTDFTLKISRVEAEDVGVYYCMQALQTPYTFGQGTKLEIK

SEQ ID NO: 9 - protein, 5 aa, synthetic construct
  source 1..5: note = J13 HCDR1
SYAMS

SEQ ID NO: 10 - protein, 17 aa, synthetic construct
  source 1..17: note = J13 HCDR2
AISGSGDSTYYADSVKG

SEQ ID NO: 11 - protein, 10 aa, synthetic construct
  source 1..10: note = J13 HCDR3
DRGFYYGMDV

SEQ ID NO: 12 - protein, 16 aa, synthetic construct
  source 1..16: note = J13 LCDR1
RSSQSLLRSNGYNYLD

SEQ ID NO: 13 - protein, 7 aa, synthetic construct
  source 1..7: note = J13 LCDR2
LGSNRAS

SEQ ID NO: 14 - protein, 9 aa, synthetic construct
  source 1..9: note = J13 LCDR3
MQALQTPFT

SEQ ID NO: 15 - protein, 119 aa, synthetic construct
  source 1..119: note = J13 VH
EVQLVESGGGLVQPGGSLRLSCVVSGFTFSSYAMSWVRQAPGKGLEWVSAISGSGDSTYYADSVKGRFTFSRDNSKNTLYLQMNSLRAEDTAVYYCTKDRGFYYGMDVWGQGTMVTVSS

SEQ ID NO: 16 - protein, 112 aa, synthetic construct
  source 1..112: note = J13 VL
DVVMTQSPLSLPVTPGEPASISCRSSQSLLRSNGYNYLDWYLQKPGQSPQLLIYLGSNRASGVPDRFSGSGSGTDFTLKISRVEAEDVGVYYCMQALQTPFTFGQGTRLEIK

SEQ ID NO: 17 - protein, 113 aa, synthetic construct
  source 1..113: note = 008QH7-J13 Vk
DVVMTQSPLSLPVTPGEPASISCRSSQSLLRSNGYNYLDWYLQKPGQSPQLLIYLGSNRASGVPDRFSGSGSGTDFTLKISRVEAEDVGVYYCMQALQTPFTFGQGTRLEIKR

SEQ ID NO: 18 - protein, 119 aa, synthetic construct
  source 1..119: note = 008QH7-J13 VH Germline
EVQLVESGGGLVQPGGSLRLSCAASGFTFSSYAMSWVRQAPGKGLEWVSAISGSGDSTYYADSVKGRFTFSRDNSKNTLYLQMNSLRAEDTAVYYCTKDRGFYYGMDVWGQGTTVTVSS

SEQ ID NO: 19 - protein, 113 aa, synthetic construct; identical to SEQ ID NO: 17
  source 1..113: note = 008QH7-J13 VL Germline

SEQ ID NO: 20-63 - skipped (44 entries)

SEQ ID NO: 64 - protein, 116 aa, synthetic construct
  source 1..116: note = 008QH7-B21_VH
QVQLQESGPGLVKPSETLSLTCTVSGGSISSYYWSWIRQPAGKGLEWIGRIYTSESTNYNPSLKSRVTMSVDTSKNQFSLKLNSVTAADTAVYYCARAPYGGLDYWGQGTLVTVSS

SEQ ID NO: 65 - protein, 107 aa, synthetic construct
  source 1..107: note = 008QH7-B21_Vk
DIQMTQSPSSLSASVGDRVTITCRASQGIRNDLGWYQQKPGKVPKLLIYAASSLQSGVPSRFSGSGSGTDFTLTISSLQPEDFATYYCLQDYNYPYTFGPGTKVDIK

SEQ ID NO: 66 - skipped

SEQ ID NO: 67 - protein, 113 aa, synthetic construct
  source 1..113: note = HT1-2108R-02-Vk_GL
DIVMTQSPLSLPVTPGEPASISCRSSQSLLHSNGYNHLDWYLQKPGQSPQLLIYLGSNRASGVPDRFSGSGSGTDFTLKISRVEAEDVGVYYCMQALQTLTWTFGQGTKVEIK

SEQ ID NO: 68 - protein, 119 aa, synthetic construct
  source 1..119: note = HT4-2108R-04-VH_GL
EVQLLESGGGLVQPGGSLRLSCAASGFTFSIYAMSWVRQAPGKGLEWVSAISGGGDSTYNADSVKGRFTISRDNSKNTLYLQMNSLRAEDTAVYYCAKDRGYYYGMVVWGQGTTVTVSS

SEQ ID NO: 69 - protein, 112 aa, synthetic construct
  source 1..112: note = HT4-2108R-38-Vk_GL
DIVMTQSPLSLPVTPGEPASISCRSSQSLLRSNGYNYLDWYLQKPGQSPQLLIYLGSNRASGVPDRFSGSGSGTDFTLKISRVEAEDVGVYYCMQALQTPYTFGQGTKLEIK

SEQ ID NO: 70 - protein, 119 aa, synthetic construct
  source 1..119: note = HT6-2108R-06-VH_GL
EVQLLESGGGLVQPGGSLRLSCAASGFTFNSYAMSWVRQAPGKGLEWVSAVSGGGDTTYYAESVKGRFTISRDNSKNTLYLQMNSLRAEDTAVYYCAKDRGYYYGMAVWGQGTTVTVSS

SEQ ID NO: 71 - protein, 112 aa, synthetic construct
  source 1..112: note = HT6-2108R-05-Vk_GL
DIVMTQSPLSLPVTPGEPASISCRSSQSLLRSNGYHYLDWYLQKPGQSPQLLIYLGSNRAPGVPDRFSGSGSGTDFTLKISRVEAEDVGVYYCMQALQTPYTFGQGTKLEIK

SEQ ID NO: 72 - protein, 119 aa, synthetic construct
  source 1..119: note = HT7-2108R-07-VH_GL
EVQLLESGGGLVQPGGSLRLSCAASGFTFSHYAMSWVRQAPGKGLEWVSAISGGGDRTFYADSVKGRFTISRDNSKNTLYLQMNSLRAEDTAVYYCAKDRGFYYGMDVWGQGTTVTVSS

SEQ ID NO: 73 - protein, 112 aa, synthetic construct
  source 1..112: note = HT7-2108R-35-Vk_GL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPYTFGQGTKLEIK

SEQ ID NO: 74 - protein, 119 aa, synthetic construct
  source 1..119: note = HT8-2108R-08-VH_GL
EVQLLESGGGLVQPGGSLRLSCAASGFTFSIYAMSWVRQAPGKGLEWVSAISGGGDTTYYADSMKGRFTISRDNSKNTLYLQMNSLRAEDTAVYYCAKDRGYYYGMDVWGQGTTVTVSS

SEQ ID NO: 75 - protein, 112 aa, synthetic construct; identical to SEQ ID NO: 73
  source 1..112: note = HT8-2108R-35-Vk_GL

SEQ ID NO: 76 - protein, 119 aa, synthetic construct
  source 1..119: note = HT10-2108R-10-VH_GL
EVQLLESGGGLVQPGGSLRLSCAASGFTFSHYAMTWVRQAPGKGLEWISAISGGGDSTYYADSVKGRFTISRDNSKNTLYLQMNSLRAEDTAVYYCAKDRGYYYGMDDWGQGTTVTVSS

SEQ ID NO: 77 - protein, 112 aa, synthetic construct; identical to SEQ ID NO: 73
  source 1..112: note = HT10-2108R-35-Vk_GL

SEQ ID NO: 78 - skipped

SEQ ID NO: 79 - protein, 107 aa, synthetic construct
  source 1..107: note = HT11-2108R-11-Vk_GL
DIQMTQSPSSLSASVGDRVTITCRASQGINNYLAWYQQKPGKVPKLLIYAASTLHSGVPSRFSGSGSATDFTLTISSLQPEDVATYYCQKYNSAPFTFGPGTKVDIK

SEQ ID NO: 80 - protein, 119 aa, synthetic construct
  source 1..119: note = HT14-2108R-14-VH_GL
EVQLLESGGGLVQPGGSLRLSCAASGFTFNIYAMTWVRQAPGKGLEWVSGISGGGDSTYYADSVKGRFTMSRDNSKNTLYLQMNSLRAEDTAVYYCAKDRGYYYGMDVWGQGTTVTVSS

SEQ ID NO: 81 - protein, 112 aa, synthetic construct
  source 1..112: note = HT14-2108R-14-Vk_GL
DIVMTQSPLSLPVTPGEPASISCKSRQSLLRSNGYNYLDWYLQKPGQSPQLLIYLGSNRASGVPDRFSGSGSGTDFTLKISRVEAEDVGVYYCMQALQTPYTFGQGTKLEIK

SEQ ID NO: 82 - protein, 119 aa, synthetic construct
  source 1..119: note = HT16-2108R-16-VH_GL
EVQLLESGGGLVQPGGSLRLSCAASGFTFSNYAMTWVRQAPGKGLEWVSAISGGGDTTYYADSVKGRFTISRDNSKNTLYLQMNSLRAEDTAVYYCAKDRGYYYGMDVWGQGTTVTVSS

SEQ ID NO: 83 - protein, 112 aa, synthetic construct; identical to SEQ ID NO: 69
  source 1..112: note = HT16-2108R-38-Vk_GL

SEQ ID NO: 84 - skipped

SEQ ID NO: 85 - protein, 107 aa, synthetic construct
  source 1..107: note = HT22-2108R-20-Vk_GL-H
EIVLTQSPATLSSSPGERATLSCRASQSVSSYLAWYQQKPGQVPRLLIYDASNRATGIPARFSGSGSGTDFTLTISNLEPEDFAVYYCQQRSSWPLTFGGGTEVEIK

SEQ ID NO: 86 - skipped

SEQ ID NO: 87 - protein, 107 aa, synthetic construct
  source 1..107: note = HT22-2108R-20-Vk_GL
EIVLTQSPATLSLSPGERATLSCRASQSVSSYLAWYQQKPGQAPRLLIYDASNRATGIPARFSGSGSGTDFTLTISSLEPEDFAVYYCQQRSSWPLTFGGGTEVEIK

SEQ ID NO: 88 - protein, 119 aa, synthetic construct
  source 1..119: note = HT28-2108R-28-VH_GL
EVQLLESGGGLVQPGGSLRLSCAASGFTFSSYAMTWVRQAPGKGLEWVSAISGGGDTTYCADSVKGRFTISRDNSKNTLYLQMNSLRAEDTAVYYYAKDRGYYYGMDVWGQGTTVTVSS

SEQ ID NO: 89 - protein, 112 aa, synthetic construct; identical to SEQ ID NO: 69
  source 1..112: note = HT28-2108R-38-Vk_GL

SEQ ID NO: 90 - protein, 119 aa, synthetic construct
  source 1..119: note = HT29-2108R-29-VH_GL
EVQLLESGGGLVQPGGSLRLSCAASGFAFSHYAMSWVRQAPGKGLEWVSAISGGGDSTYYADSVKGRFTISRDNSKNTLYLQMNSLRAEDTAVYYCAKDRGYYYGMDVWGQGTTVTVSS

SEQ ID NO: 91 - protein, 112 aa, synthetic construct; identical to SEQ ID NO: 69
  source 1..112: note = HT29-2108R-38-Vk_GL

SEQ ID NO: 92 - protein, 119 aa, synthetic construct
  source 1..119: note = HT30-2108R-30-VH_GL
EVQLLESGGGLVQPGGSLRLSCAASGFTFSIYAMSWVRQAPGKGLEWVSAITAGGDTTYYAVSVKGRFTISRDNSKNTLYLQMNSLRAEDTAVYYCAKDRGYYYGMDVWGQGTTVTVSS

SEQ ID NO: 93 - protein, 112 aa, synthetic construct; identical to SEQ ID NO: 73
  source 1..112: note = HT30-2108R-35-Vk_GL

SEQ ID NO: 94 - protein, 119 aa, synthetic construct
  source 1..119: note = HT31-2108R-31-VH_GL
EVQLLESGGGLVQPGGSLRLSCAASGFTFSIYAMSWVRQAPGKGLEWVSAISGGGDSTYYADSVKGRFTISRDNSKNTLYLQMNSLRAEDTAVYYCAKDRGFYYGMDVWGQGTTVTVSS

SEQ ID NO: 95 - protein, 112 aa, synthetic construct; identical to SEQ ID NO: 69
  source 1..112: note = HT31-2108R-38-Vk_GL

SEQ ID NO: 96 - protein, 119 aa, synthetic construct
  source 1..119: note = HT32-2108R-32-VH_GL
EVQLLESGGGLVQPGGSLRLSCAASGFTFSSYAMSWVRQAPGKGLEWVSAISGGGDTTYYADSVKGRFTISRDNSKNTLYLQMNSLRAEDTAVYYCAKDRGFYYGMDVWGQGTTVTVSS

SEQ ID NO: 97 - protein, 112 aa, synthetic construct; identical to SEQ ID NO: 69
  source 1..112: note = HT32-2108R-38-Vk_GL

SEQ ID NO: 98 - protein, 119 aa, synthetic construct
  source 1..119: note = HT33-2108R-33-VH_GL
EVQLLESGGGLVQPGGSLRLSCAASGFTFGHYAMSWVRQAPGKGLEWVSAISGGGDYTYYADSVKGRLTISRDNSKNTLYLQMNSLRAEDTAVYYCAKDRGYYYGMDVWGQGTTVTVSS

SEQ ID NO: 99 - protein, 112 aa, synthetic construct
  source 1..112: note = HT33-2108R-33-Vk_GL
DIVMTQSPLSLPVTPGEPASISCRSSQSLLHSNGYKYLDWYLQKPGQSPQLLIYLGSNRASGVPDRFSGSGSGTDFTLKISRVEAEDVGVYYCMQALQIPYTFGQGTKLEIK

SEQ ID NO: 100 - protein, 119 aa, synthetic construct
  source 1..119: note = HT34-2108R-34-VH_GL-L
EVQLLESGGGLVQPGGSLRLSCAASGFTFSHYAMTWVRQAPGKGLEWVSAISGGGDTTYYADSVKGRFTISRDNSKTTLYLQMNSLRAEDTAVYYCAKDRGYYYGMDVWGQGTTVTVSS

SEQ ID NO: 101 - protein, 112 aa, synthetic construct
  source 1..112: note = HT34-2108R-34-Vk_GL-L
DIVMTQSPLSLPVTPGEPASISCRSGQSLLRSNGYNYLDWYLQKPGQSPQLLIYLGSNRASGVPDRFSGSGSGTDFTLKISRVEAEDVGVYYCMQALQTPFTFGPGTKVDIK

SEQ ID NO: 102 - protein, 119 aa, synthetic construct
  source 1..119: note = HT34-2108R-34-VH_GL
EVQLLESGGGLVQPGGSLRLSCAASGFTFSHYAMTWVRQAPGKGLEWVSAISGGGDTTYYADSVKGRFTISRDNSKNTLYLQMNSLRAEDTAVYYCAKDRGYYYGMDVWGQGTTVTVSS

SEQ ID NO: 103 - protein, 112 aa, synthetic construct; identical to SEQ ID NO: 101
  source 1..112: note = HT34-2108R-34-Vk_GL

SEQ ID NO: 104 - protein, 119 aa, synthetic construct
  source 1..119: note = HT35-2108R-35-VH_GL
EVQLLESGGGLVQPGGSLRLSCAASGFTFSHYAMTWVRQAPGKGLEWISAISGGGDSTYYADSVKGRFTISRDNSKNTLYLQMNSLRAEDTAVYYCAKDRGYYYGMDVWGQGTTVTVSS

SEQ ID NO: 105 - protein, 112 aa, synthetic construct; identical to SEQ ID NO: 73
  source 1..112: note = HT35-2108R-35-Vk_GL

SEQ ID NO: 106 - protein, 119 aa, synthetic construct
  source 1..119: note = HT36-2108R-36-VH_GL
EVQLLESGGGLVQPGGSLRLSCAASGFTFSIYAMSWVRQAPGKGLEWVSAISGGGDTTYYADSVKGRFTSSRDNSKNTLYLQMNSLRAEDTAVYFCAKDRGFYYGMDVWGQGTTVTVSS

SEQ ID NO: 107 - protein, 112 aa, synthetic construct
  source 1..112: note = HT36-2108R-36-Vk_GL
DIVMTQSPLSLPVTPGEPASISCRSSQSLLRSNGYIYLDWYLQKPGQSPQLLIYLGSNRASGVPDRFSGSGSGTDFTLKISRVEAEDVGVYYCMQALQTPYTFGQGTKLEIK

SEQ ID NO: 108 - protein, 119 aa, synthetic construct
  source 1..119: note = HT37-2108R-37-VH_GL-L
EVQLLESGGGLVQPGGSLRLSCAASGFTFSIYAMTWVRQAPGKGLEWVSAISAGGDNTYNADSVKGRFTISRDNSKITLYLQMNSLRAEDTAVYYCAKDRGYYYGMDVWGQGTTVTVSS

SEQ ID NO: 109 - protein, 112 aa, synthetic construct; identical to SEQ ID NO: 69
  source 1..112: note = HT37-2108R-38-Vk_GL-L

SEQ ID NO: 110 - protein, 119 aa, synthetic construct
  source 1..119: note = HT37-2108R-37-VH_GL
EVQLLESGGGLVQPGGSLRLSCAASGFTFSIYAMTWVRQAPGKGLEWVSAISAGGDNTYNADSVKGRFTISRDNSKNTLYLQMNSLRAEDTAVYYCAKDRGYYYGMDVWGQGTTVTVSS

SEQ ID NO: 111 - protein, 112 aa, synthetic construct; identical to SEQ ID NO: 69
  source 1..112: note = HT37-2108R-38-Vk_GL

SEQ ID NO: 112 - protein, 119 aa, synthetic construct
  source 1..119: note = HT38-2108R-38-VH_GL
EVQLLESGGGLVQPGGSLRLSCAASGFTFSSYAMSWVRQAPGKGLEWVSAISGGGDSTYYTDSVKGRFTISRDNSKNTLYLQMNSLRAEDTAVYYCAKDRGFYYGMDVWGQGTTVTVSS

SEQ ID NO: 113 - protein, 112 aa, synthetic construct; identical to SEQ ID NO: 69
  source 1..112: note = HT38-2108R-38-Vk_GL

SEQ ID NO: 114 - protein, 119 aa, synthetic construct; identical to SEQ ID NO: 112
  source 1..119: note = HT38-2108R-38-VH_GL-Q

SEQ ID NO: 115 - protein, 112 aa, synthetic construct
  source 1..112: note = HT38-2108R-38-Vk_GL-Q
DIVMTQSPLSLPVTPGEPASISCRSSQSLLRSQGYNYLDWYLQKPGQSPQLLIYLGSNRASGVPDRFSGSGSGTDFTLKISRVEAEDVGVYYCMQALQTPYTFGQGTKLEIK

SEQ ID NO: 116 - protein, 449 aa, synthetic construct
  source 1..449: note = HT12-GL Heavy Chain
EVQLLESGGGLVQPGGSLRLSCAASGFTFNIYAMTWVRQAPGKGLEWVSGISAGGDSTYYADSVKGRFTISRDNSKNTLYLQMNSLRAEDTAVYYCAKDRGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 219 aa, synthetic construct
  source 1..219: note = HT12-GL Light Chain
DIVMTQSPLSLPVTPGEPASISCRSRQSLLRSNGYNYLDWYLQKPGQSPQLLIYLGSNRASGVPDRFSGSGSGTDFTLKISRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - DNA, 1347 bp, synthetic construct
  source 1..1347: mol_type = other DNA; note = HT12-GL Heavy Chain nucleotides
gaggttcagcttctggaatctgggggcggtcttgtacagccggggggctcccttcgcctgtcttgtgccgcatctgggttcacgtttaatatctatgcaatgacgtgggtgcgacaagcgcctgggaaaggattggagtgggtgtcagggatttctgctggaggtgattcaacatactacgcggatagtgtcaaaggcagattcacgataagccgggataattcaaaaaacacgctttacctgcaaatgaactccttgcgggctgaagacacagcagtctattactgtgcgaaggatcggggctactactacgggatggatgtttggggccaggggacaaccgtcacggtctcatccgcgtcgaccaagggcccatccgtcttccccctggcaccctcctccaagagcacctctgggggcacagcggccctgggctgcctggtcaaggactacttccccgaaccggtgacggtgtcctggaactcaggcgctctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtctacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 119 - DNA, 657 bp, synthetic construct
  source 1..657: mol_type = other DNA; note = HT12-GL Light Chain nucleotides
gacatcgttatgacccaaagtcctctctcactccccgttacccctggagaaccagcaagcattagttgcaggagccgacagagcctgttgcggtctaatggctataattatctggattggtatctgcagaaacctggtcaatcaccacagcttctcatatatctgggaagcaaccgcgcttccggtgtgccagaccgcttctcaggatctggttctggcacagatttcacgttgaaaatcagccgagttgaagcggaggatgtgggtgtctattactgcatgcaagcactgcagacaccatatacgttcggccaaggaacaaaactggaaattaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2025/0051463 A1
(43) Pub. Date: Feb. 13, 2025

(54) CD123 ANTIBODY-DRUG CONJUGATES AND METHODS OF USING THE SAME

(71) Applicant: ASTRAZENECA AB, Södertälje (SE)

(72) Inventors: Dipannita DUTTA, Gaithersburg, MD (US); Ryan FLEMING, Gaithersburg, MD (US); Pan PAN, Gaithersburg, MD (US); Marcio ANDRADE CAMPOS, Cambridge (GB); Casey DANIELS, Gaithersburg, MD (US); Gullu GORGUN, Waltham, MA (US)

(21) Appl. No.: 18/785,065

(22) Filed: Jul. 26, 2024

Related U.S. Application Data
(60) Provisional application No. 63/635,835, filed on Apr. 18, 2024.
(60) Provisional application No. 63/607,876, filed on Dec. 8, 2023.
(60) Provisional application No. 63/516,678, filed on Jul. 31, 2023.

(51) Int. Cl. C07K 16/28; A61K 31/635; A61K 31/706; A61K 47/68; A61P 35/02

(52) U.S. Cl. CPC C07K 16/2866; A61K 31/635; A61K 31/706; A61K 47/68037; A61K 47/6849; A61K 47/6889; A61P 35/02; C07K 2317/569; C07K 2317/622

(57) ABSTRACT

The present disclosure generally relates to antibodies, antigen binding fragments thereof, polypeptides, and immunoconjugates that bind to CD123 antigen (the α chain of the interleukin-3 receptor) and deliver a payload to CD123 expressing cells. The payload is a topoisomerase inhibitor that efficiently kills CD123 expressing tumor cells while sparing hematopoietic stem cells and mature hematopoietic cells.

### CROSS-REFERENCE TO RELATED APPLICATIONS

This application claims priority from U.S. provisional application Nos. 63/516,678 filed Jul. 31, 2023; 63/607,876 filed Dec. 8, 2023; and 63/635,835 filed Apr. 18, 2024, each of which are incorporated herein by reference.

### REFERENCE TO SEQUENCE LISTING

The content of the electronically submitted sequence listing (Name: CD123-100-WO-PCT_ST26.xml, Size: 90.9 KB, and Date of Creation: Apr. 11, 2024) submitted in this application is incorporated herein by reference in its entirety.

### BACKGROUND OF THE DISCLOSURE

Antibody drug conjugates (ADC) have emerged as a powerful tool in the fight against various types of diseases, including cancer. The antibody component of ADC mediates selective targeting of specific cells and a cytotoxic drug component allows the selective killing of the targeted cell. However, efficiency of target cell killing and ADC stability remain issues to be addressed.

CD123 is expressed in hematologic cancers with limited expression in normal hematopoietic cells. CD123 has been targeted by antibody drug conjugates (ADC), e.g., a CD123-alkylator ADC (e.g., pivekimab sunirine), but low stability and high toxicity require the use of low doses with low response rates. Therefore, improved CD123-based therapies are needed.

### SUMMARY OF THE DISCLOSURE

Provided is an antibody or antigen binding fragment thereof comprising an antigen binding domain that comprises: (i) a variable heavy chain region (VH) comprising a VH complementarity determining region (CDR) 1 comprising an amino acid sequence selected from SEQ ID NOs: 1 and 9, a VH-CDR2 comprising an amino acid sequence selected from SEQ ID NOs: 2 and 10, and a VH-CDR3 comprising an amino acid sequence selected from SEQ ID NOs: 3 and 11, and (ii) a variable light chain region (VL) comprising a VL-CDR1 comprising an amino acid sequence selected from SEQ ID NOs: 4 and 12, a VL-CDR2 comprising an amino acid sequence selected from SEQ ID NOs: 5 and 13, and a VL-CDR3 comprising an amino acid sequence selected from SEQ ID NOs: 6 and 14.

In some aspects, the antibody or antigen binding fragment thereof described herein specifically binds CD123.

In some aspects, the antigen binding-domain comprises a Fab, Fab′, F(ab′)2, Fd, Fv, single-chain fragment variable (scFv), single chain antibody, VHH, vNAR, nanobody (single-domain antibody), or any combination thereof.

In some aspects, the antigen binding domain comprises a scFv.

In some aspects, the antigen binding domain comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antigen binding domain comprises a VH-CDR1 comprising the amino acid sequence set forth in SEQ ID NO: 9, a VH-CDR2 comprising the amino acid sequence set forth in SEQ ID NO: 10, a VH-CDR3 comprising the amino acid sequence set forth in SEQ ID NO: 11, a VL-CDR1 comprising the amino acid sequence set forth in SEQ ID NO: 12, a VL-CDR2 comprising the amino acid sequence set forth in SEQ ID NO: 13, and a VL-CDR3 comprising the amino acid sequence set forth in SEQ ID NO: 14.

In some aspects, the antigen binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7 and 15.

In some aspects, the antigen binding domain comprises a VH comprising an amino acid sequence selected from SEQ ID NOs: 7 and 15.

In some aspects, the antigen binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8 and 16.

In some aspects, the antigen binding domain comprises a VL comprising an amino acid sequence selected from SEQ ID NOs: 8 and 16.

In some aspects, the antigen binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8.

In some aspects, the antigen binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 15, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16.

In some aspects, the antigen binding domain comprises a VH comprising an amino acid sequence set forth in SEQ ID NO: 7 and a VL comprising an amino acid sequence set forth in SEQ ID NO: 8.

In some aspects, the antigen binding domain comprises a VH comprising an amino acid sequence set forth in SEQ ID NO: 15 and a VL comprising an amino acid sequence set forth in SEQ ID NO: 16.

Further provided is an antibody or antigen binding fragment thereof comprising an antigen binding domain that comprises: a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NO: 64, 68, 70, 72, 74, 76, 80, 82, 88, 90, 92, 94, 96, 98, 100, 102, 104, 106, 108, 110, 112, and 114; and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NO: 65, 67, 69, 71, 73, 75, 77, 79, 81, 83, 85, 89, 91, 93, 95, 97, 99, 101, 103, 105, 107, 109, 111, 113, and 115.

In some aspects, the antigen binding domain of the antibody or antigen binding fragment comprises:
a VH comprising an amino acid sequence set forth in SEQ ID NO: 64 and a VL comprising an amino acid sequence set forth in SEQ ID NO: 65;
a VH comprising an amino acid sequence set forth in SEQ ID NO: 68 and a VL comprising an amino acid sequence set forth in SEQ ID NO: 69;
a VH comprising an amino acid sequence set forth in SEQ ID NO: 70 and a VL comprising an amino acid sequence set forth in SEQ ID NO: 71;
a VH comprising an amino acid sequence set forth in SEQ ID NO: 72 and a VL comprising an amino acid sequence set forth in SEQ ID NO: 73;
a VH comprising an amino acid sequence set forth in SEQ ID NO: 74 and a VL comprising an amino acid sequence set forth in SEQ ID NO: 75;
a VH comprising an amino acid sequence set forth in SEQ ID NO: 76 and a VL comprising an amino acid sequence set forth in SEQ ID NO: 77;
a VH comprising an amino acid sequence set forth in SEQ ID NO: 80 and a VL comprising an amino acid sequence set forth in SEQ ID NO: 81;
a VH comprising an amino acid sequence set forth in SEQ ID NO: 82 and a VL comprising an amino acid sequence set forth in SEQ ID NO: 83;
a VH comprising an amino acid sequence set forth in SEQ ID NO: 88 and a VL comprising an amino acid sequence set forth in SEQ ID NO: 89;
a VH comprising an amino acid sequence set forth in SEQ ID NO: 90 and a VL comprising an amino acid sequence set forth in SEQ ID NO: 91;
a VH comprising an amino acid sequence set forth in SEQ ID NO: 92 and a VL comprising an amino acid sequence set forth in SEQ ID NO: 93;
a VH comprising an amino acid sequence set forth in SEQ ID NO: 94 and a VL comprising an amino acid sequence set forth in SEQ ID NO: 95;
a VH comprising an amino acid sequence set forth in SEQ ID NO: 96 and a VL comprising an amino acid sequence set forth in SEQ ID NO: 97;
a VH comprising an amino acid sequence set forth in SEQ ID NO: 98 and a VL comprising an amino acid sequence set forth in SEQ ID NO: 99;
a VH comprising an amino acid sequence set forth in SEQ ID NO: 100 and a VL comprising an amino acid sequence set forth in SEQ ID NO: 101;
a VH comprising an amino acid sequence set forth in SEQ ID NO: 102 and a VL comprising an amino acid sequence set forth in SEQ ID NO: 103;
a VH comprising an amino acid sequence set forth in SEQ ID NO: 104 and a VL comprising an amino acid sequence set forth in SEQ ID NO: 105;
a VH comprising an amino acid sequence set forth in SEQ ID NO: 106 and a VL comprising an amino acid sequence set forth in SEQ ID NO: 107;
a VH comprising an amino acid sequence set forth in SEQ ID NO: 108 and a VL comprising an amino acid sequence set forth in SEQ ID NO: 109;
a VH comprising an amino acid sequence set forth in SEQ ID NO: 110 and a VL comprising an amino acid sequence set forth in SEQ ID NO: 111;
a VH comprising an amino acid sequence set forth in SEQ ID NO: 112 and a VL comprising an amino acid sequence set forth in SEQ ID NO: 113; or
a VH comprising an amino acid sequence set forth in SEQ ID NO: 114 and a VL comprising an amino acid sequence set forth in SEQ ID NO: 115.

In some aspects, the antibody binding domain, e.g., the antibody or antibody binding fragment, comprises a VH comprising an amino acid sequence set forth in SEQ ID NO: 64 and a VL comprising an amino acid sequence set forth in SEQ ID NO: 65.

In some aspects, the antibody binding domain, e.g., the antibody or antibody binding fragment, comprises a VH comprising an amino acid sequence set forth in SEQ ID NO: 68 and a VL comprising an amino acid sequence set forth in SEQ ID NO: 69.

In some aspects, the antibody binding domain, e.g., the antibody or antibody binding fragment, comprises a VH comprising an amino acid sequence set forth in SEQ ID NO: 70 and a VL comprising an amino acid sequence set forth in SEQ ID NO: 71.

In some aspects, the antibody binding domain, e.g., the antibody or antibody binding fragment, comprises a VH comprising an amino acid sequence set forth in SEQ ID NO: 72 and a VL comprising an amino acid sequence set forth in SEQ ID NO: 73.

In some aspects, the antibody binding domain, e.g., the antibody or antibody binding fragment, comprises a VH comprising an amino acid sequence set forth in SEQ ID NO: 74 and a VL comprising an amino acid sequence set forth in SEQ ID NO: 75.

In some aspects, the antibody binding domain, e.g., the antibody or antibody binding fragment, comprises a VH comprising an amino acid sequence set forth in SEQ ID NO: 76 and a VL comprising an amino acid sequence set forth in SEQ ID NO: 77.

In some aspects, the antibody binding domain, e.g., the antibody or antibody binding fragment, comprises a VH comprising an amino acid sequence set forth in SEQ ID NO: 80 and a VL comprising an amino acid sequence set forth in SEQ ID NO: 81.

In some aspects, the antibody binding domain, e.g., the antibody or antibody binding fragment, comprises a VH comprising an amino acid sequence set forth in SEQ ID NO: 82 and a VL comprising an amino acid sequence set forth in SEQ ID NO: 83.

In some aspects, the antibody binding domain, e.g., the antibody or antibody binding fragment, comprises a VH comprising an amino acid sequence set forth in SEQ ID NO: 88 and a VL comprising an amino acid sequence set forth in SEQ ID NO: 89.

In some aspects, the antibody binding domain, e.g., the antibody or antibody binding fragment, comprises a VH comprising an amino acid sequence set forth in SEQ ID NO: 90 and a VL comprising an amino acid sequence set forth in SEQ ID NO: 91.

In some aspects, the antibody binding domain, e.g., the antibody or antibody binding fragment, comprises a VH comprising an amino acid sequence set forth in SEQ ID NO: 92 and a VL comprising an amino acid sequence set forth in SEQ ID NO: 93.

In some aspects, the antibody binding domain, e.g., the antibody or antibody binding fragment, comprises a VH comprising an amino acid sequence set forth in SEQ ID NO: 94 and a VL comprising an amino acid sequence set forth in SEQ ID NO: 95.

In some aspects, the antibody binding domain, e.g., the antibody or antibody binding fragment, comprises a VH comprising an amino acid sequence set forth in SEQ ID NO: 96 and a VL comprising an amino acid sequence set forth in SEQ ID NO: 97.

In some aspects, the antibody binding domain, e.g., the antibody or antibody binding fragment, comprises a VH comprising an amino acid sequence set forth in SEQ ID NO: 98 and a VL comprising an amino acid sequence set forth in SEQ ID NO: 99.

In some aspects, the antibody binding domain, e.g., the antibody or antibody binding fragment, comprises a VH comprising an amino acid sequence set forth in SEQ ID NO: 100 and a VL comprising an amino acid sequence set forth in SEQ ID NO: 101.

In some aspects, the antibody binding domain, e.g., the antibody or antibody binding fragment, comprises a VH comprising an amino acid sequence set forth in SEQ ID NO: 102 and a VL comprising an amino acid sequence set forth in SEQ ID NO: 103.

In some aspects, the antibody binding domain, e.g., the antibody or antibody binding fragment, comprises a VH comprising an amino acid sequence set forth in SEQ ID NO: 104 and a VL comprising an amino acid sequence set forth in SEQ ID NO: 105.

In some aspects, the antibody binding domain, e.g., the antibody or antibody binding fragment, comprises a VH comprising an amino acid sequence set forth in SEQ ID NO: 106 and a VL comprising an amino acid sequence set forth in SEQ ID NO: 107.

In some aspects, the antibody binding domain, e.g., the antibody or antibody binding fragment, comprises a VH comprising an amino acid sequence set forth in SEQ ID NO: 108 and a VL comprising an amino acid sequence set forth in SEQ ID NO: 109.

In some aspects, the antibody binding domain, e.g., the antibody or antibody binding fragment, comprises a VH comprising an amino acid sequence set forth in SEQ ID NO: 110 and a VL comprising an amino acid sequence set forth in SEQ ID NO: 111.

In some aspects, the antibody binding domain, e.g., the antibody or antibody binding fragment, comprises a VH comprising an amino acid sequence set forth in SEQ ID NO: 112 and a VL comprising an amino acid sequence set forth in SEQ ID NO: 113.

In some aspects, the antibody binding domain, e.g., the antibody or antibody binding fragment, comprises a VH comprising an amino acid sequence set forth in SEQ ID NO: 114 and a VL comprising an amino acid sequence set forth in SEQ ID NO: 115.

Further provided is a polynucleotide encoding an antibody or antigen binding fragment thereof described herein.

Also provided is a cell comprising a polynucleotide described herein.

Further provided is a conjugate of formula I: A-(DL)ₚ or a pharmaceutically acceptable salt or solvate thereof, wherein A is an antibody or antigen binding fragment thereof as described herein and DL is a Drug Linker unit that is of formula II:

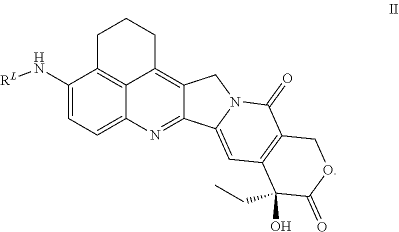

In some aspects, R^{L} is a linker for connection to the antibody or antigen binding fragment thereof, wherein R^{L} is of formula IIa:

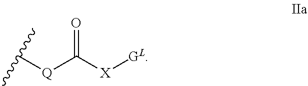

In some aspects, Q of formula IIa is:

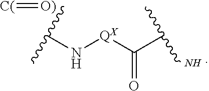

In some aspects, Q^{X} of Q is such that Q is an amino acid residue, a dipeptide residue, a tripeptide residue or a tetrapeptide residue, wherein the superscripted labels ^{C(═O)} and ^{NH} indicate the group to which the atoms are bound.

In some aspects, X of formula IIa is:

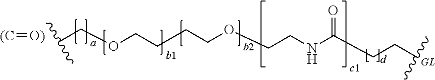

In some aspects, in X, a is from 0 to 5, b1 is from 0 to 16, b2 is from 0 to 16, c1 is 0 or 1, d is from 0 to 5; and GL is a linker for connecting to the antibody or antigen binding fragment thereof as described herein.

In some aspects, Q of formula IIa is an amino acid residue selected from: Phe, Lys, Val, Ala, Cit, Leu, Ile, Arg, and Trp.

In some aspects, Q of formula IIa is a dipeptide residue selected from: NH-Phe-Lys-C═O, NH-Val-Ala-C═O, NH-Val-Lys-C═O, NH-Ala-Lys-C═O, NH-Val-Cit-C═O, NH-Phe-Cit-C═O, NH-Leu-Cit-C═O, NH-Ile-Cit-C═O, NH-Phe-Arg-C═O, NH-Trp-Cit-C═O, and NH-Gly-Val-C═O.

In some aspects, Q of formula IIa is a tripeptide residue selected from: NH-Glu-Val-Ala-C═O, NH-Glu-Val-Cit-C═O, NH-αGlu-Val-Ala-C═O, and NH-αGlu-Val-Cit-C═O.

In some aspects, Q of formula IIa is a tetrapeptide residue selected from: NH-Gly-Gly-Phe-Gly-C═O; and NH-Gly-Phe-Gly-Gly-C═O, wherein NH represents the N-terminus, and C═O represents the C-terminus of the residue.

In some aspects, a of X is 0 to 3; 0 or 1.

In some aspects, b1 of X is 0 to 8; 0; 2; 3; 4; 5; or 8.

In some aspects, b2 of X is 0 to 8; 2; 3; 4; 5; or 8.

In some aspects, c1 of X is 0; 1; or 2.

In some aspects, d of X is 0 to 3; 0, 1, or 2.

In some aspects, a is 0, b1 is 0, c1 is 1, d is 2, and b2 is 0, 2, 3, 4, 5, or 8.

In some aspects, a is 1, b2 is 0, c1 is 0, d is 0, and b1 is 0, 2, 3, 4, 5, or 8.

In some aspects, a is 0, b1 is 0, c1 is 0, d is 1, and b2 is 0, 2, 3, 4, 5, or 8.

In some aspects, b1 is 0, b2 is 0, c1 is 0, one of a and d is 0, and the other of a and d is 1 or 5.

In some aspects, a is 1, b2 is 0, c1 is 0, d is 2, and b1 is 0, 2, 3, 4, 5, or 8.

In some aspects, a is 0, b1 is 0, b2 is 8, c1 is 1, and d is 0.

In some aspects, GL is

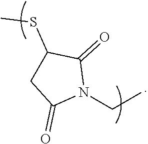

In some aspects, Q is NH-Val-Ala-C═O.

In some aspects, a of X is 1. In some aspects, b1 of X is 8. In some aspects, c1 of X is 1. In some aspects, d of X is 1. In some aspects, a is 0. In some aspects, b1 is 0. In some aspects, c1 is 1. In some aspects, d is 0. In some aspects, a is 0, b1 is 0, b2 is 8, c1 is 1, and d is 0.

In some aspects, formula IIa is:

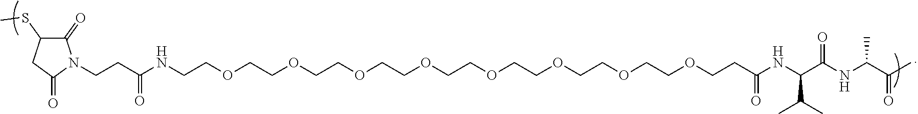

In some aspects, a CD123 binding antibody as described herein is an IgG antibody.

In some aspects, at least one DL unit is bound to a hinge region of the IgG antibody.

In some aspects, one to three DL units are bound to a heavy chain of the IgG antibody.

In some aspects, one DL unit is bound to a light chain of the IgG antibody.

In some aspects, one to three DL units are bound to each heavy chain of the IgG antibody and one DL unit is bound to each light chain of the IgG antibody.

In some aspects, formula II is a single enantiomer or in an enantiomerically enriched form.

In some aspects, p of formula I is an integer of from 1 to 20. In some aspects, p is an integer of from 1 to about 10. In some aspects, p is 8.

In some aspects, the antibody or antigen binding fragment thereof described herein is conjugated to

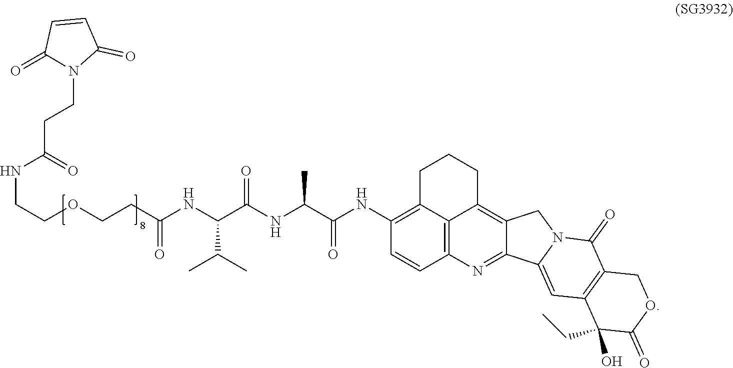

Also provided is a mixture of conjugates as described herein, wherein an average drug loading per antibody or antigen binding fragment thereof in the mixture of conjugates is about 1 to about 10.

Provided is a pharmaceutical composition comprising a conjugate or a mixture of conjugates and a pharmaceutically acceptable diluent, carrier or excipient.

Provided is also a polynucleotide sequence encoding an antibody or antigen binding fragment thereof described herein.

In some aspects, the disclosure provides a method of producing an antibody or antigen binding fragment thereof that specifically binds to CD123 comprising culturing the cell described herein in a suitable condition.

In some aspects, the disclosure provides a method of producing the conjugate or the mixture of conjugates described herein comprising conjugating the antibody or antigen binding fragment thereof to the Drug Linker unit.

In some aspects, the disclosure provides a method of treating a proliferative disease, the method comprising administering to a subject in need thereof a therapeutically effective amount of the antibody or antigen binding fragment thereof, the polynucleotide, the vector, or the cell described herein.

Further provided is a method of treating a proliferative disease, the method comprising administering to a subject in need thereof a therapeutically effective amount of a conjugate, a mixture, or a pharmaceutical composition described herein. In some aspects, the proliferative disease is cancer.

In some aspects, the cancer is a hematologic cancer. In some aspects, the hematologic cancer is a leukemia or lymphoma. In some aspects, the cancer is acute myeloid leukemia, acute lymphoid leukemia, myelodysplastic syndrome, refractory anemia with excess blasts, DLBCL non-Hodgkin lymphoma, marginal zone non-Hodgkin lymphoma, mantle zone non-Hodgkin lymphoma, follicular non-Hodgkin lymphoma, Hodgkin lymphoma, or minimal residual disease.

Further provided is a conjugate, a mixture of conjugates, or a pharmaceutical composition described herein for use in medical treatment.

Further provided is a conjugate, a mixture, or a pharmaceutical composition described herein for use in the treatment of a proliferative disease.

In some aspects, the conjugate, mixture or pharmaceutical composition is for use in the treatment of cancer.

Also provided is a use of a conjugate, a mixture, or a pharmaceutical composition described herein in the manufacture of a medicament for the treatment of a proliferative disease.

In some aspects, the present disclosure provides an antibody-drug conjugate (ADC) comprising: (i) antibody or antigen binding fragment thereof which binds to CD123 comprising: a HCDR1 comprising the amino acid sequence as set forth in SEQ ID NO: 1; a HCDR2 comprising the amino acid sequence as set forth in SEQ ID NO: 2; a HCDR3 comprising the amino acid sequence as set forth in SEQ ID NO: 3; and a LCDR1 comprising the amino acid sequence as set forth in SEQ ID NO: 4; a LCDR2 comprising the amino acid sequence as set forth in SEQ ID NO: 5; and a LCDR3 comprising the amino acid sequence as set forth in SEQ ID NO: 6;

(ii) one or more cysteine residues of the antibody or antigen binding fragment covalently bound to a linker-payload, through a succinimidyl thioether, of formula:

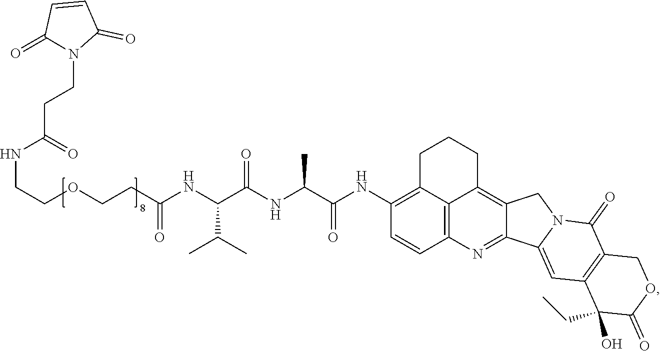

and wherein the ADC has a drug to antibody ratio (DAR) of about 8. In some aspects, the ADC comprises a variable heavy (VH) chain comprising the amino acid sequence as set forth in SEQ ID NO: 7 and a variable light (VL) chain comprising the amino acid sequence as set forth in SEQ ID NO: 8. In some aspects, the ADC comprises a heavy chain (HC) comprising the amino acid sequence as set forth in SEQ ID NO: 116, and a light chain (LC) comprising the amino acid sequence as set forth in SEQ ID NO: 117. In some aspects, the disclosure provides a pharmaceutical composition comprising the ADC. In some aspects, the present disclosure provides a method of treating a cancer comprising administering the ADC to a patient. In some aspects, the cancer is acute myeloid leukemia or myelodyslplastic syndrome. In some aspects the disclosure provides the ADC, or the pharmaceutical composition described herein for use in the treatment of cancer. In some aspects, the cancer is acute myeloid leukemia or myelodysplastic syndrome. In some aspects, the methods described herein further comprise administering venetoclax and/or a hypomethylating agent, optionally wherein the hypomethylating agent is 5-azacytidine or decitabine

### BRIEF DESCRIPTION OF THE FIGURES

FIG. 1A shows a flow cytometric analysis of live gated healthy donor bone marrow mononuclear cells (BMMCs) and AML patient BMMCs. FIG. 1B shows percent expression of CD33 and CD123 on healthy donor and AML BMMC cells. FIG. 1C shows CD123 antibody binding capacity (ABC) on hematopoietic cells of healthy donors and AML patients leukemic populations. FIG. 1D shows high frequency of CD123+ cells in AML patient bone marrow compared to healthy donor bone marrow by Flow Cytometry. FIG. 1E shows AML patient bone marrow with increased CD123 expression in lysates by western blotting. FIG. 1F shows high prevalence of CD123 positivity in AML patient bone marrow (right panel) by immunohistochemistry compared to normal bone marrow (left panel).

FIG. 2A shows a schematic of a CD123 antibody topoisomerase inhibitor (Top1i) drug conjugate (ADC) and cysteine conjugation sites according to EU numbering. FIG. 2B shows the drug moiety, and linker-payload, before and after enzymatic release of the active Top1i payload from the linker-payload.

FIG. 3A shows binding curves of sensor bound HT12-GL antibody and human IL3Ra (CD123) sample. FIG. 3B shows binding curves of sensor bound HT12-GL antibody and human IL-5Ra sample. FIG. 3C shows binding curves of sensor bound HT12-GL antibody and GM-CSFRa sample. FIG. 3D shows binding curves of sensor bound J13 antibody and human CD123 sample. FIG. 3E shows binding curves of sensor bound HT12-GL antibody and human CD123 sample. FIG. 3F shows binding curves of sensor bound J13 antibody and cynomolgus CD123 sample. FIG. 3G shows binding curves of sensor bound HT12-GL antibody and cynomolgus CD123 sample.

FIG. 4A shows HT12-GL antibody binding capacity across AML cell lines. FIG. 4B shows binding affinity of HT12-GL antibodies to CD123 expressed in different species. FIG. 4C shows HT12-GL antibody binding to EOL-1 cells compared to control antibody NIP228. FIG. 4D shows reduced or no binding of various HT antibody clones to CD123-negative 293 cells. FIG. 4E shows binding of various HT antibody clones to 293 cells expressing human CD123. FIG. 4F shows binding of various HT antibody clones to 293 cells expressing cynomolgus CD123. FIG. 4G shows binding of various HT antibody clones to Mv411 cells. FIG. 4H shows binding of various HT antibody clones to OCI-AML5 cells. FIG. 4I shows lack of binding of various HT antibody clones to CD123-negative Jurkat cells. FIG. 4J shows binding of HT and various 008Q antibody clones to 293 cells expressing human CD123. FIG. 4K shows binding of HT and various 008Q antibody clones to 293 cells expressing cynomolgus CD123. FIG. 4L shows lack of binding of HT and various 008Q antibody clones to CD123-negative Jurkat cells.

FIG. 5A shows specific receptor expression dependent binding of HT12-GL antibody to doxycycline (+dox) induced, high CD123 expressing THP-1 cells, moderate binding to THP-1 cells expressing moderate CD123 expression and no binding to CD123 negative Hel92.1.7 cells. FIG. 5B shows microscopy images of internalization of HT12-GL in puncta (marked with arrows) into CD123 expressing THP-1 cells. FIG. 5C shows internalization and lysosomal trafficking of HT12-GL antibody and HT12-GL ADC (HT12-GL-SG3932) over time compared to control NIP228 antibody and NIP228 ADC into CD123 expressing THP-1 cells by flow based methods. FIG. 5D shows internalization and lysosomal trafficking of HT12-GL antibody compared to control NIP228 antibody into Molm13 cells over time. FIG. 5E shows internalization of various HT antibody clones into Molm13 cells.

FIG. 6A shows the ability of HT12-GL and some CD123 antibodies to inhibit IL-3 mediated cell proliferation. FIG. 6B shows GM-CSF did not affect TF-1 cell proliferation in the presence of CD123 antibodies. FIG. 6C shows the concentrations of HT12-GL antibodies needed to inhibit IL-3 mediated cell proliferation. FIG. 6D shows inhibition of IL-3 mediated STAT5 phosphorylation by HT12-GL antibodies (CD123 MAb) and HT12-GL ADC ((HT12-GL-SG3932)).

FIG. 7A shows cytotoxic killing of CD123 expressing cells by HT12-GL ADC (HT12-GL-SG3932) and not by Isotype NIP228 ADC. FIG. 7B shows no killing of CD123 negative Hel92.1.7 cells by HT12-GL ADC (HT12-GL-SG3932). FIG. 7C shows increased caspase 3/7 activity in CD123 expressing cells in the presence of HT12-GL ADC (HT12-GL-SG3932). FIG. 7D shows no caspase 3/7 activity in CD123-negative cells in the presence of HT12-GL ADC (HT12-GL-SG3932). FIG. 7E shows flow cytometric images of THP-1 cells (CLEC GFP) with low CD123 expression. FIG. 7F shows flow cytometric images of high CD123 expressing THP-1 cells (CLEC DOX). FIG. 7G shows flow cytometric images of co-cultures of both THP-1 CLEC-GFP and THP-1 CLEC DOX cells. FIG. 7H shows bystander killing of THP-1 cells (CLEC GFP) in the presence of HT12-GL ADC with increasing amounts of CD123 high expressing THP-1 cells (CLEC DOX). FIG. 7I shows a western blot analysis of DNA damage response signalling proteins induced by HT12-GL ADC treatment.

FIG. 8A shows relative lack of killing of CD123-negative Ad293 cells in the presence of various HT antibody ADCs (HT-SG3932). FIG. 8B shows killing of Ad293 cells ectopically expressing humanCD123 in the presence of various HT antibody ADCs (HT-SG3932) compared to a control NIP228 ADCs. FIG. 8C shows killing of Ad293 cells ectopically expressing cynomolgus CD123 in the presence of various HT antibody ADCs compared to a control NIP228 ADCs. FIG. 8D shows relative lack of killing of CD123 low THP-1 cells in the presence of various HT antibody ADCs and control NIP228 ADC. FIG. 8E shows killing of dox-induced line CD123 high expressing THP-1 cells in the presence of various HT antibody ADCs compared to control NIP228 ADC.

FIG. 9A shows potency of HT12-GL ADC (HT12-GL-SG3932) compared to CD33 ADC Mylotarg and isotype ADC in CFU assays of healthy donor bone marrow and AML patient bone marrow. FIG. 9B shows total cell numbers following a 6-day treatment with HT12-GL ADC (HT12-GL-SG3932) of healthy donor hematopoietic stem cells (HSCs: Lineage−CD34⁺CD38⁻ CD90⁺CD45RA⁻), granulocyte-monocyte progenitor cells (GMP:Lineage−CD34⁺CD38⁺CD135⁺CD45RA⁺), and differentiated cells (Lineage⁺) in bone marrow CD34+ BMMCs expansion assays. FIG. 9C shows relative lack of toxicity of HT12-GL ADC ((HT12-GL-SG3932)) towards HUVEC compared to HT12-GL mAb, control ADC (NIP228 TOPO ADC), and Comparator-1 ADC. FIG. 9D shows relative lack of killing of HT12-GL ADC ((HT12-GL-SG3932)) towards HPAEC compared to HT12-GL mAb, control ADC (NIP228 TOPO ADC), and Comparator-1 ADC. FIG. 9E shows relative lack of killing of HT12-GL ADC ((HT12-GL-SG3932)) towards RPMI-8226. FIG. 9F shows relative lack of killing of HT12-GL ADC ((HT12-GL-SG3932)) towards K562. FIG. 9G shows a western blot measuring CD123 in RPMI-8226, K562, HPAEC and HUVEC cell lines. FIG. 9H shows surface expression of CD123 measured by flow cytometry on RPMI-8226, HUVEC, HPAEC, and K562 cell lines.

FIG. 10A shows tumor volume in a subcutaneous Mv4-11 xenograft model in untreated SCID mice and SCID mice treated with HT12-GL ADC (HT12-GL-SG3932) at increasing concentrations or isotype antibody ADC. FIG. 10B shows tumor volume in a subcutaneous Mv4-11 xenograft model in untreated SCID mice and SCID mice treated with 2 mg/kg HT12-GL ADC ((HT12-GL-SG3932)), J13 ADC (J13-SG3932), or an isotype control antibody ADC (NIP-228). FIG. 10C shows tumor volume in a subcutaneous EOL xenograft model in untreated Nude mice and Nude mice treated with HT12-GL antibody (CD123 mAb), HT12-GL ADC (HT12-GL-SG3932) or isotype antibody ADC. FIG. 10D shows tumor volume in a subcutaneous EOL xenograft model in untreated Nude mice and Nude mice treated with HT12-GL antibody (CD123 mAb), HT12-GL ADC (HT12-GL-SG3932), or isotype antibody ADC.

FIG. 11A shows survival in a disseminated Mv4-11 xenograft model in untreated mice and mice treated with 5 mg/kg HT12-GL ADC (HT12) (HT12-GL-SG3932), J13 ADC (J13-SG3932), or an isotype antibody ADC (NTP228). FIG. 11B shows survival in a disseminated Mv4-11 xenograft model in untreated mice and mice treated with a single dose or 2 doses of 5 mg/kg HT12-GL ADC or an isotype antibody ADC.

FIG. 12A shows the characterization of AML Patient-Derived Xenograft (PDX) models. FIG. 12B shows the anti-leukemic efficacy of 5 mg/kg HT12-GL ADC (HT12-GL-SG3932) in reducing CD45+ cells in whole blood of disseminated AML PDX models. FIG. 12C shows the anti-leukemic efficacy of 5 mg/kg HT12-GL ADC (HT12-GL-SG3932) in reducing CD45+ cells in bone marrow of disseminated AML PDX models. FIG. 12D shows the anti-leukemic efficacy of 10 mg/kg HT12-GL ADC (HT12-GL-SG3932) in reducing CD45+ cells in whole blood of disseminated AML PDX models. FIG. 12E shows the anti-leukemic efficacy of 10 mg/kg HT12-GL ADC (HT12-GL-SG3932) in reducing CD45+ cells in bone marrow of disseminated AML PDX models. FIG. 12F shows the anti-leukemic efficacy of 5 mg/kg and 10 mg/kg HT12-GL ADC (HT12-GL-SG3932) in reducing human CD123+ cells in bone marrow of disseminated AML PDX models.

FIG. 13A shows total antibody concentrations of HT12-GL ADC (HT12-GL-SG3932) measured by ELISA versus LCMS. FIG. 13B shows HT12-GL ADC (HT12-GL-SG3932) antibody and payload stability measured by LCMS. FIG. 13C shows the stability of HT12-GL heavy and light chains. FIG. 13D shows payload stability of HT12-GL ADC.

FIG. 14A shows stability of HT12-GL ADC (HT12-GL-SG3932), J13 ADC (J13-SG3932), and in mouse serum. FIG. 14B shows stability of HT12-GL ADC (HT12-GL-SG3932), and J13 ADC (J13-SG3932) in cynomolgus serum. FIG. 14C shows stability of HT12-GL ADC (HT12-GL-SG3932), and J13 ADC (J13-SG3932) in rat serum.

FIG. 15A shows cytoxic synergy as a percentage of cell viability between HT12-GL-SG3932 and venetoclax (VEN) in both THP-1 and OCI-AML3 AML cell lines in vitro. FIG. 15B shows cytoxic synergy as a percentage of cell viability between HT12-GL-SG3932 and HMA (decitabine) in both THP-1 and OCI-AML3 AML cell lines in vitro. FIG. 15C shows cytoxic synergy as a percentage of cell viability between HT12-GL-SG3932 and SoC (VEN and Decitabine) in both THP-1 and OCI-AML3 AML cell lines in vitro.

FIG. 16 shows cytoxic synergy as a percentage of cell viability between HT12-GL-SG3932 and Cytarabine in both THP-1 and Mv411 AML cell lines in vitro.

FIG. 17 shows a summary heat map of synergy scores between HT12-GL-SG3932 and VEN, HT12-GL-SG3932 and Decitabine, HT12-GL-SG3932 and Cytarabine, and HT12-GL-SG3932 and SoC (VEN and Decitabine) in 8 AML cell lines tested. Data was analyzed in Rstudio and combination benefit was assessed using the Synergyfinder package in R. Bliss scores≥5 concurrent with <50% cell viability was interpreted to represent meaningful synergy.

FIG. 18A shows drug sensitivity analysis as a percentage of blasts normalized to control (untreated) at 4 days post-treatment in 8 of the 11 AML patient samples identified as Top1i sensitive. FIG. 18B shows drug sensitivity analysis as a percentage of blasts normalized to control (untreated) at 4 days post-treatment in 3 of the 11 AML patient samples identified as Top1i insensitive. FIG. 18C shows drug sensitivity analysis 7 days post-treatment in 8 of the 11 AML patient samples identified as Top1i sensitive. FIG. 18D shows drug sensitivity analysis 7 days post-treatment in 3 of the 11 AML patient samples identified as Top1i insensitive.

FIG. 19 shows survival of a SoC-insensitive AML PDX (DFAM-68555) model after treatment as shown in the table as Groups 1-8.

FIG. 20 shows the AML disease burden in a SoC-insensitive AML PDX (DFAM-68555) model on 16 days post-first dose of treatment as measured by % hCD45⁺ cells in the peripheral blood.

FIG. 21 shows survival of a SoC-sensitive AML PDX (DFAM-49600) model after treatment as shown in the table as Groups 1-8.

FIG. 22 shows the AML disease burden in a SoC-insensitive AML PDX (DFAM-68555) model on 16, 31, 44, 59, 73, 87, 100, and 146 days post-first dose of treatment as measured by % hCD45⁺ cells in the peripheral blood.

### DETAILED DESCRIPTION OF THE DISCLOSURE

The present disclosure relates to antibodies, antigen binding fragments thereof, and antibody drug conjugates that bind to CD123 antigen (the α chain of the interleukin-3 receptor, or IL3Rα). The antibody drug conjugates comprise a topoisomerase inhibitor derivative drug unit that is connected to an antibody or antigen binding fragment described herein through a linker. In some aspects, the linker is cleavable and releases the drug unit in a cellular environment after uptake of the antibody drug conjugate into a target cell. The released topoisomerase inhibitor mediates cell killing of the target cell and bystander killing of non-target cells. Further provided are polynucleotides encoding the CD123 binding antibodies or antigen binding fragments, vectors for expressing the same in host cells and methods of making and using the CD123 antibody drug conjugates for cancer therapy.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the antibody to host cells, tissues, or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system.

The “Kabat numbering system” is generally used when referring to a residue in the variable domain (approximately residues 1-107 of the light chain and residues 1-113 of the heavy chain) (e.g., Kabat et al., Sequences of Immunological Interest, 5th Ed. Public Health Service, National Institutes of Health, Bethesda, Md. (1991)).

The amino acid position numbering as in Kabat, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82.

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop, when numbered using the Kabat numbering convention, varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. The table below lists the positions of the amino acids comprising the variable regions of the antibodies in each system.

| Region | Kabat | AbM | Chothia |
|---|---|---|---|
| LCDR1 | L24-L34 | L24-L34 | L24-L34 |
| LCDR2 | L50-L56 | L50-L56 | L50-L56 |
| LCDR3 | L89-L97 | L89-L97 | L89-L97 |
| HCDR1¹ | H31-H35B | H26-H35B | H26-H32 . . . 34 |
| HCDR1² | H31-H35 | H26-H35 | H26-H32 |
| HCDR2 | H50-H65 | H50-H58 | H52-H56 |
| HCDR3 | H95-H102 | H95-H102 | H95-H102 |

¹Kabat Numbering
²Chothia Numbering

ImMunoGeneTics (IMGT) also provides a numbering system for the immunoglobulin variable regions, including the CDRs. See, e.g., Lefranc, M. P. et al., Dev. Comp. Immunol. 27: 55-77(2003). The IMGT numbering system is based on an alignment of more than 5,000 sequences, structural data, and characterization of hypervariable loops and allows for easy comparison of the variable and CDR regions for all species. According to the IMGT numbering schema, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

As used throughout the specification the VH CDRs sequences described correspond to the classical Kabat numbering locations, namely Kabat VH-CDR1 is at positions 31-35, VH-CDR2 is a positions 50-65, and VH-CDR3 is at positions 95-102. VL-CDR1, VL-CDR2 and VL-CDR3 also correspond to classical Kabat numbering locations, namely positions 24-34, 50-56 and 89-97, respectively.

An antibody can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In some aspects, the antibodies described herein are of the IgG1 subtype. Antibodies, e.g., IgG1 exist in several allotypes, which differ from each other in at most a few amino acids. Antibodies described herein include, by way of example, both naturally-occurring and non-naturally-occurring antibodies; monoclonal and polyclonal antibodies; chimeric and humanized antibodies; human and nonhuman antibodies and wholly synthetic antibodies.

The term “monoclonal antibody,” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al (1975) Nature 256:495, or may be made by recombinant DNA methods (see, U.S. Pat. No. 4,816,567). The monoclonal antibodies may also be isolated from phage antibody libraries using the techniques described in Clackson et al (1991) Nature, 352:624-628; Marks et al (1991) J. Mol. Biol., 222:581-597 or from transgenic mice carrying a fully human immunoglobulin system (Lonberg (2008) Curr. Opinion 20(4):450-459). The monoclonal antibodies herein specifically include chimeric antibodies, humanized antibodies and human antibodies.

The term “antigen binding fragment” of an antibody, as used herein, refers to one or more fragments of an antibody that retain the ability to specifically bind to an antigen (e.g., human CD123). The antigen binding function of an antibody can be performed by fragments of a full-length antibody. Examples of binding fragments encompassed within the term “antigen binding fragment” of an antibody, e.g., an anti-CD123 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen binding fragment” of an antibody. These antibody fragments are obtained using conventional techniques known to those skilled in the art, and the fragments are screened for utility in the same manner as are intact antibodies. Antigen binding fragments can be produced by recombinant DNA techniques, or by enzymatic or chemical cleavage of intact antibodies.

As used herein, the term “affinity” refers to a measure of the strength of the binding of an antigen or target (such as an epitope) to its cognate binding domain (such as a paratope). As used herein, the term “avidity” refers to the overall stability of the complex between a population of epitopes and paratopes (i.e., antigens and antigen binding domains).

The term “epitope” refers to a site on an antigen (e.g., CD123) to which an antibody or antigen binding fragment specifically binds, e.g., as defined by the specific method used to identify it. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation.

The term “binds to the same epitope” with reference to two or more antigen binding moieties means that the antigen binding moieties bind to the same segment of amino acid residues. Antigen binding moieties that “compete with another antibody for binding to a target” refer to antigen binding moieties that inhibit (partially or completely) the binding of the other antibody to the target.

The terms “specific binding,” “selective binding,” “selectively binds,” and “specifically binds,” as used herein, refer to an antigen binding moiety (e.g., an antibody or antigen binding fragment) binding to an epitope on a predetermined antigen. Typically, the antigen binding moiety (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 instrument using the predetermined antigen, e.g., human CD123, as the analyte and the antibody as the ligand, or Scatchard analysis of binding of the antibody to antigen positive cells, and (ii) binds to the predetermined antigen with an affinity that is at least two-fold greater than its affinity for binding to an antigen other than the predetermined antigen (e.g., BSA, casein) or a closely-related antigen. Accordingly, an antigen binding moiety (e.g., an antibody or antigen binding fragment) that “specifically binds to human CD123” refers to an antigen binding moiety that binds to human CD123 with a K_{D} of 10⁻⁷ M or less, such as approximately less than 10⁻⁸ M, 10⁻⁹ M or 10⁻¹⁰ M or even lower.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., a NK cell.

The term “immunotherapy,” as used herein refers to the treatment of a subject afflicted with, or at risk of contracting or suffering a recurrence of, a disease by a method comprising inducing, enhancing, suppressing or otherwise modifying the immune system or an immune response.

The term “polypeptide,” as used herein, is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with, any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, disulfide bond formation, proteolytic cleavage, or modification by non-naturally occurring amino acids. The term “peptide,” as used herein, encompasses full length peptides and fragments, variants or derivatives thereof. A “peptide” as disclosed herein can be part of a fusion polypeptide comprising additional components such as, e.g., an albumin or PEG moiety, to increase half-life. A peptide as described herein can be derivatized in a number of different ways. A peptide described herein can comprise modifications including e.g., conjugation of a palmitoyl group. A “peptide” as disclosed herein can also be part of a drug linker unit that covalently connects an antibody or antigen binding fragment thereof to a drug to form an antibody drug conjugate.

The phrase “conservative amino acid substitutions,” as used herein, refers to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in a CD123-binding moiety (e.g., an anti-CD123 antibody or CD123 binding fragment) is replaced with another amino acid residue from the same side chain family.

The terms “polynucleotide,” and “nucleic acid molecule,” as used herein, are intended to include DNA molecules and RNA molecules. A polynucleotide or nucleic acid molecule can be single-stranded or double-stranded, and can be cDNA.

The term “promoter,” as used herein, refers to a DNA sequence recognized by the machinery of a cell, or introduced synthetic machinery, required to initiate the specific transcription of a gene. The term “promoter” is also meant to encompass those nucleic acid elements sufficient for promoter-dependent gene expression controllable for cell-type specific, tissue-specific or inducible expression by external signals or agents; such elements can be located in the 5′ or 3′ regions of the native gene. In some aspects, the promoter can be a constitutively active promoter, a cell-type specific promoter, or an inducible promoter.

The term “IRES,” as used herein, refers to an element that promotes direct internal ribosome entry to the initiation codon, such as ATG, of a cistron (a protein encoding region), thereby leading to the cap-independent translation of the gene. See, e.g., Jackson R J et al., Trends Biochem Sci 15(12):477-83 (199); Jackson R J and Kaminski, A. RNA 1(10):985-1000 (1995). Under translational control of an IRES translation proceeds in a cap-independent manner.

The term “termination signal sequence,” as used herein, can be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

The terms “operatively linked,” “operatively inserted,” “operatively positioned,” “under control” or “under transcriptional control,” as used herein, mean that the promoter is in the correct location and orientation in relation to the nucleic acid to control RNA polymerase initiation and expression of the gene. The term “operably linked” means that a DNA sequence and a regulatory sequence(s) are connected in such a way as to permit gene expression when the appropriate molecules (e.g., transcriptional activator proteins) are bound to the regulatory sequence(s). The term “operably inserted” means that the DNA of interest introduced into a cell is positioned adjacent a DNA sequence which directs transcription and translation of the introduced DNA (i.e., facilitates the production of, e.g., a polypeptide encoded by a DNA of interest).

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using several known algorithms including the algorithm of E. Meyers and W. Miller (CABIOS, 4: 11-17 (1989)), which has been incorporated into the ALIGN program (version 2.0); the Needleman and Wunsch (J. Mol. Biol. (48):444-453 (1970)) algorithm, which has been incorporated into the GAP program in the GCG software package.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

The term “vector,” as used herein, refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present disclosure, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant cell,” “recombinant host cell” (or simply “host cell”), as used herein, refers to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “recombinant cell” or “host cell” as used herein.

The term “antibody drug conjugate,” as used herein refers to a molecule that commonly comprises three distinct elements: a cell binding agent (e.g., an antibody or antigen binding fragment); a linker; and a cytotoxic drug moiety. In some aspects, the cytotoxic drug moiety is covalently attached to an antibody or antigen binding fragment through one or more lysine residues on the antibody or antigen binding fragment, or to one or more cysteine residues on the antibody or antigen binding fragment thereof. In some aspects, two or more cysteine residues can be obtained in an antibody or antigen binding fragment through reduction of an inter-chain disulfide bond. In some aspects, one or more cysteine residues can be obtained in an antibody or antigen binding fragment through introduction of one or more mutations into the polynucleotide encoding the antibody or antigen binding fragment thereof. For example, a non-cysteine amino acid can be mutated to a cysteine or a cysteine can be added to the amino acid sequence of the antibody or antigen binding fragment. In some aspects, a lysine can be chemically converted to a thiol group. Included in the term “antibody drug conjugate” are heterogeneous mixtures containing antibodies or antigen binding fragments with various numbers of drugs attached, and antibodies or antigen binding fragments with drugs attached at different positions on the antibody or antigen binding fragment.

The term “linker,” as used herein refers to any chemical moiety that is capable of linking a compound, e.g., a drug as described herein to a cell-binding agent, e.g., a CD123 antibody or antigen binding fragment thereof as described herein in a stable, covalent manner. Linkers can be susceptible to or be substantially resistant to acid-induced cleavage, light-induced cleavage, peptidase-induced cleavage, esterase-induced cleavage, and disulfide bond cleavage, at conditions under which the drug or the antibody or antigen binding fragment remains active. Suitable linkers are well known in the art and include, for example, peptide linkers, disulfide groups, thioether groups, acid labile groups, photolabile groups, peptidase labile groups and esterase labile groups. Linkers also include charged linkers and hydrophilic forms thereof as described herein and know in the art.

The term “peptide linker,” as used herein, refers to a peptide comprising one or more amino acids that covalently connect a drug as described herein to an antibody or antigen binding fragment thereof as described herein either directly or through an additional linker group. A peptide linker can comprise a dipeptide, tripeptide, tetrapeptide or longer peptide chain.

The term “cancer,” as used herein, refers a broad group of diseases characterized by the uncontrolled growth of abnormal cells in the body. Unregulated cell division can result in the formation of malignant tumors or cells that invade neighboring tissues and can metastasize to distant parts of the body through the lymphatic system or bloodstream.

The terms “subject,” “individual,” or “patient,” as used herein, refer to any organism to which a composition disclosed herein, e.g., an antibody, antigen binding fragment, or antibody drug conjugate (ADC) of the present disclosure, can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition.

The terms “treat,” “treated,” and “treating,” as used herein, refer to both therapeutic treatment and prophylactic or preventative measures wherein the object is to prevent or slow down (lessen) an undesired physiological, e.g., a cancer or obtain beneficial or desired clinical results. In some aspects, treating reduces or lessens the symptoms associated with, e.g., cancer. In some aspects, the treating results in a beneficial or desired clinical result. Beneficial or desired clinical results include, but are not limited to, alleviation of symptoms; diminishment of the extent of a condition, disorder, or disease; attainment of a stabilized (i.e., not worsening) state of a condition, disorder, or disease; delay in onset or slowing of a condition, disorder, or disease progression; amelioration of a condition, disorder, or disease state or remission (whether partial or total), whether detectable or undetectable; amelioration of at least one measurable physical parameter, not necessarily discernible by the patient; or enhancement or improvement of a condition, disorder, or disease. In some aspects, treatment includes eliciting a clinically significant response without excessive levels of side effects. In some aspects, treatment includes prolonging survival as compared to expected survival if not receiving treatment. As used herein, the term “amelioration” or “ameliorating” refers to a lessening of severity of at least one indicator of a condition or disease. As used herein, the term “preventing” or “prevention” refers to delaying or forestalling the onset, development or progression of a condition or disease for a period of time, including weeks, months, or years. Ameliorating the disease or disorder includes slowing the course of the disease or disorder or reducing the severity of symptoms of the disease or disorder.

The terms “effective amount,” “therapeutically effective amount,” and a “sufficient amount” of, e.g., an antibody, antigen binding fragment thereof, an antibody drug conjugate or a composition disclosed herein refer to a quantity sufficient to, when administered to a subject including a human effect beneficial or desired results, including alleviation of symptoms; diminishment of the extent of a condition, disorder, or disease; attainment of a stabilized (i.e., not worsening) state of a condition, disorder, or disease; delay in onset or slowing of a condition, disorder, or disease progression; amelioration of a condition, disorder, or disease state or remission (whether partial or total), whether detectable or undetectable; amelioration of at least one measurable physical parameter, not necessarily discernible by a patient; or enhancement or improvement of a condition, disorder, or disease. In some aspects, treatment includes eliciting a clinically significant response without excessive levels of side effects. As such, a “therapeutically effective amount” or synonyms thereof depends on the context in which it is applied. In some aspects, a therapeutically effective amount of an agent (e.g., an antibody, antigen binding fragment, an antibody drug conjugate or a composition described herein) is an amount that results in a beneficial or desired result in a subject as compared to a control that does not receive the agent. The amount of a given agent (e.g., an antibody, antigen binding fragment, an antibody drug conjugate or a composition) will vary depending upon various factors, such as the given agent, the pharmaceutical formulation, the route of administration, the type of disease or disorder, the identity of the subject (e.g., age, sex, and/or weight) being treated, and the like.

The term “prophylactically effective amount,” as used herein, refers to an amount of an agent (e.g., an antibody, antigen binding fragment, an antibody drug conjugate or a composition) that delays, forestalls the onset, or blocks the onset or development or progression of a condition or disease for a period of time, including weeks, months, or years. The prophylactically effective amount can vary depending on the characteristics of an agent (e.g., an antibody, antigen binding fragment, an antibody drug conjugate or a composition); how the agent is administered; the degree of risk of disease; and the history, age, weight, family history, genetic makeup of a subject; the types of preceding or concomitant treatments, if any; and other individual characteristics of the patient to be treated.

As used herein, the terms “ug” and “uM” are used interchangeably with “μg” and “μM,” respectively.

The term “topoisomerase inhibitors, as used herein, refers to chemical compounds that block the action of topoisomerase (topoisomerase I and II), which is a type of enzyme that controls the changes in DNA structure by catalyzing the breaking and rejoining of the phosphodiester backbone of DNA strands during the normal cell cycle.

The phrase “superscripted labels ^{C(═O)} and ^{NH}” as used herein, for example in the formula

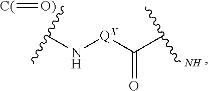

refers to the group to which the atoms are bound. For example, the NH group in the formula above is shown as being bound to a carbonyl (superscript ^{C(═O)}) which is not part of the moiety illustrated; and the carbonyl group in the formula above is shown as being bound to a NH group (superscript ^{NH}), which is not part of the moiety illustrated.

The term “chiral,” as used herein, refers to molecules, which have the property of non-superimposability of the mirror image partner, while the term “achiral” refers to molecules which are superimposable on their mirror image partner.

The term “stereoisomers,” as used herein, refers to drugs, which have identical chemical constitution, but differ with regard to the arrangement of the atoms or groups in space.

The term “diastereomer,” as used herein, refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

The term “enantiomers,” as used herein, refers to two stereoisomers of a drug which are non-superimposable mirror images of one another. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Drugs”, John Wiley & Sons, Inc., New York, 1994. The drugs of the disclosure may contain asymmetric or chiral centers, and therefore exist in different stereoisomeric forms. It is intended that all stereoisomeric forms of the drugs of the disclosure, including but not limited to, diastereomers, enantiomers and atropisomers, as well as mixtures thereof such as racemic mixtures, form part of the present disclosure. Many organic drugs exist in optically active forms, i.e., they have the ability to rotate the plane of plane-polarized light. In describing an optically active drug, the prefixes D and L, or R and S, are used to denote the absolute configuration of the molecule about its chiral center(s). The prefixes d and l or (+) and (−) are employed to designate the sign of rotation of plane-polarized light by the drug, with (−) or l meaning that the drug is levorotatory. A drug prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “enantiomerically enriched form,” as used herein, refers to a sample of a chiral substance whose enantiomeric ratio is greater than 50:50 but less than 100:0. Except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇alkyl includes n propyl and iso-propyl; butyl includes n-, iso-, sec-, and tert-butyl; methoxyphenyl includes ortho-, meta-, and para-methoxyphenyl). The above exclusion does not pertain to tautomeric forms, for example, keto-, enol-, and enolate-forms, as in, for example, the following tautomeric pairs: keto/enol (illustrated below), imine/enamine, amide/imino alcohol, amidine/enediamine, nitroso/oxime, thioketone/enethiol, N nitroso/hyroxyazo, and nitro/aci-nitro.

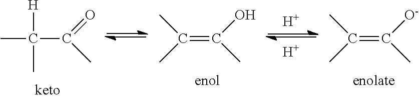

The terms “tautomer” or “tautomeric form,” as used herein, refer to structural isomers of different energies, which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons. Specifically included in the term “isomer” are drugs with one or more isotopic substitutions. For example, H may be in any isotopic form, including 1H, 2H (deuterium, D), and 3H (tritium, T); C may be in any isotopic form, including 12C, 13C, and 14C; O may be in any isotopic form, including 16O and 18O; and the like. Examples of isotopes that can be incorporated into drugs of the disclosure include isotopes of hydrogen, carbon, nitrogen, oxygen, phosphorous, fluorine, chlorine and iodine, such as, but not limited to 2H, 3H, 12C, 13C, 14C, 15N, 18F, 31P, 32P, 35S, 36Cl, and 125I. Various isotopically labeled drugs of the present disclosure include, for example those into which radioactive isotopes such as 3H, 13C, and 14C are incorporated. Such isotopically labelled drugs may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labeled or substituted therapeutic drugs of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life, reduced dosage requirements, or an improvement in therapeutic index. It is understood that deuterium in this context is regarded as a substituent. An 18F labeled drug may be useful for PET or SPECT studies. Isotopically labeled drugs of the disclosure and prodrugs thereof can generally be prepared by substituting a readily available isotopically labeled reagent for a non-isotopically labeled reagent. The concentration of such a heavier isotope, specifically deuterium, may be defined by an isotopic enrichment factor. In the drugs of this disclosure any atom not specifically designated as a particular isotope is meant to represent any stable isotope of that atom.

Unless otherwise specified, a reference to a particular drug includes all such isomeric forms, including (wholly or partially) racemic and other mixtures thereof. Methods for the preparation (e.g. asymmetric synthesis) and separation (e.g. fractional crystallization and chromatographic means) of such isomeric forms are either known in the art or are readily obtained by adapting the methods taught in WO 2020/200880, incorporated herein in its entirety.

### II. Antibodies and Antigen Binding Fragments Thereof

Provided are antibodies or antigen binding fragments thereof that specifically bind human CD123 (IL3Rα). In some aspects, the antibodies or antigen binding fragments thereof comprise a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3.

In some aspects, the antibodies or antigen binding fragments thereof comprise a VH-CDR3 comprising an amino acid sequence comprising SEQ ID NO: 3 or 11. In some aspects, the antibodies or antigen binding fragments thereof comprise a VH-CDR2 comprising an amino acid sequence comprising SEQ ID NO: 2 or 10. In some aspects, the antibodies or antigen binding fragments thereof comprise a VH-CDR1 comprising an amino acid sequence comprising SEQ ID NO: 1 or 9.

In some aspects, the antibodies or antigen binding fragments thereof comprise a VL-CDR3 comprising an amino acid sequence comprising SEQ ID NO: 6 or 14. In some aspects, the antibodies or antigen binding fragments thereof comprise a VL-CDR2 comprising an amino acid sequence comprising SEQ ID NO: 5 or 13. In some aspects, the antibodies or antigen binding fragments thereof comprise a VL-CDR1 comprising an amino acid sequence comprising SEQ ID NO: 4 or 12.

In some aspects, the antibody or antigen binding fragment thereof comprises a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the antibody or antigen binding fragment thereof comprises a VH-CDR1 comprising the amino acid sequence set forth in SEQ ID NO: 9, a VH-CDR2 comprising the amino acid sequence set forth in SEQ ID NO: 10, a VH-CDR3 comprising the amino acid sequence set forth in SEQ ID NO: 11, a VL-CDR1 comprising the amino acid sequence set forth in SEQ ID NO: 12, a VL-CDR2 comprising the amino acid sequence set forth in SEQ ID NO: 13, and a VL-CDR3 comprising the amino acid sequence set forth in SEQ ID NO: 14.

In some aspects, the antibody or antigen binding fragment thereof comprises the VH-CDR1, VH-CDR2, and VH-CDR3 present in the VH region comprising the amino acid sequence set forth in SEQ ID NO: 7; and the VL-CDR1, VL-CDR2, and VL-CDR3 present in the VL region comprising the amino acid sequence set forth in SEQ ID NO: 8.

In some aspects, the antibody or antigen binding fragment thereof comprises the VH-CDR1, VH-CDR2, and VH-CDR3 present in the VH region comprising the amino acid sequence set forth in SEQ ID NO: 15; and the VL-CDR1, VL-CDR2, and VL-CDR3 present in the VL region comprising the amino acid sequence set forth in SEQ ID NO: 16.

In some aspects, the antibody or antigen binding fragment thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence comprising SEQ ID NO: 7 or 15. In some aspects, the antibody or antigen binding fragment thereof comprises a VH comprising an amino acid sequence comprising SEQ ID NO: 7. In some aspects, the antibody or antigen binding fragment thereof comprises a VH comprising an amino acid sequence comprising SEQ ID NO: 15.

In some aspects, the antibody or antigen binding fragment thereof comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence comprising SEQ ID NO: 8 or 16. In some aspects, the antibody or antigen binding fragment thereof comprises a VL comprising an amino acid sequence comprising SEQ ID NO: 8. In some aspects, the antibody or antigen binding fragment thereof comprises a VL comprising an amino acid sequence comprising SEQ ID NO: 16.

In some aspects, the antibody or antigen binding fragment thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the antibody or antigen binding fragment thereof comprises a VH comprising the amino acid sequence set forth in SEQ ID NO: 7, and a VL comprising the amino acid sequence set forth in SEQ ID NO: 8.

In some aspects, the antibody or antigen binding fragment thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 15, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16. In some aspects, the antibody or antigen binding fragment thereof comprises a VH comprising the amino acid sequence set forth in SEQ ID NO: 15, and a VL comprising the amino acid sequence set forth in SEQ ID NO: 16.

In some aspects, the antibody or antigen binding fragment thereof cross competes for binding to human CD123 with an antibody or antigen binding fragment thereof disclosed herein. In some aspects, the antibody or antigen binding fragment thereof binds the same epitope on human CD123 as an antibody or antigen binding fragment thereof disclosed herein. In some aspects, the antibody or antigen binding fragment thereof binds an overlapping epitope on human CD123 as an antibody or antigen binding fragment thereof disclosed herein.

In some aspects, an antibody or antigen binding fragment thereof described herein binds to CD123 with a high affinity. In some aspects, an antibody or antigen binding fragment thereof described herein binds to CD123 with a K_{D} of about 10⁻⁷ M or less. In some aspects, an antibody or antigen binding fragment thereof described herein binds to CD123 with a K_{D} of about 10⁻⁸ M, about 10⁻⁹ M, about 10⁻¹⁰ M, about 10⁻¹¹ M, or lower.

In some aspects, an antibody or antigen binding fragment thereof described herein binds to CD123 in an in vitro binding assay with a K_{D} of between about 5×10⁻⁸ M, 1×about 10⁻⁸M, about 5×10⁻⁹ M, about 1×10⁻⁹ M, about 5×10⁻¹⁰ M, about 1×10⁻¹⁰ M, about 5×10⁻¹¹ M about, or about 1×10⁻¹¹ M. In some aspects, an antibody or antigen binding fragment thereof described herein binds to human CD123 in an in vitro binding assay with a K_{D} of between about 9×10⁻⁹ M and about 1.7×10⁻¹⁰ M. In some aspects, an antibody or antigen binding fragment thereof described herein binds to human CD123 in an in vitro binding assay with a K_{D} of about 9×10⁻⁹ M or about 1.7×10⁻¹⁰ M. In some aspects, an antibody or antigen binding fragment thereof described herein binds to cynomolgus CD123 in an in vitro binding assay with a K_{D} of between about 1.2×10⁻⁸M and about 6.5×10⁻¹⁰ M. In some aspects, an antibody or antigen binding fragment thereof described herein binds to cynomolgus CD123 in an in vitro binding assay with a K_{D} of about 1.2×10⁻⁸ M or about 6.5×10⁻¹⁰ M.

In some aspects, an antibody or antigen binding fragment thereof described herein competes with IL3 for binding to an IL3Rα. In some aspects, an antibody or antigen binding fragment thereof described herein inhibits IL3 mediated cell proliferation. In some aspects, an antibody or antigen binding fragment thereof described herein inhibits IL3Rα mediated cell signaling.

### Antibody Preparation

The antibodies of the present disclosure can be obtained using conventional techniques known to persons skilled in the art and their utility confirmed by conventional binding studies—an exemplary method is described in Example 4. By way of example, a simple binding assay is to incubate the cell expressing an antigen with the antibody. If the antibody is tagged with a fluorophore, the binding of the antibody to the antigen can be detected by FACS analysis.

Antibodies of the present disclosure can be raised in various animals including mice, rats, rabbits, goats, sheep, monkeys or horses. Antibodies may be raised following immunisation with individual capsular polysaccharides, or with a plurality of capsular polysaccharides. Blood isolated from these animals contains polyclonal antibodies—multiple antibodies that bind to the same antigen. Antigens may also be injected into chickens for generation of polyclonal antibodies in egg yolk. To obtain a monoclonal antibody that is specific for a single epitope of an antigen, antibody-secreting lymphocytes are isolated from an animal and immortalized by fusing them with a cancer cell line. The fused cells are called hybridomas, and will continually grow and secrete antibody in culture. Single hybridoma cells are isolated by dilution cloning to generate cell clones that all produce the same antibody; these antibodies are called monoclonal antibodies. Methods for producing monoclonal antibodies are conventional techniques known to those skilled in the art (see e.g. Making and Using Antibodies: A Practical Handbook. GC Howard. CRC Books. 2006. ISBN 0849335280). Polyclonal and monoclonal antibodies are often purified using Protein A/G or antigen-affinity chromatography.

The antibody or antigen binding fragment thereof of the disclosure can be prepared as a monoclonal anti-CD123 antibody, which can be prepared using hybridoma methods, such as those described by Kohler and Milstein, Nature 256:495 (1975). Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or an in vitro binding assay, e.g., radioimmunoassay (RIA) or enzyme-linked immunosorbent assay (ELISA), can then be propagated either in in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid using known methods.

Alternatively, the antibody or antigen binding fragment thereof (e.g. as monoclonal antibodies) can also be made using recombinant DNA methods as described in U.S. Pat. No. 4,816,567. The polynucleotides encoding a monoclonal antibody are isolated from mature B-cells or hybridoma cell, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or antigen binding fragments thereof of the desired species can be isolated from phage display libraries expressing CDRs of the desired species as described in McCafferty et al., Nature 348:552-554 (1990); Clackson et al., Nature, 352:624-628 (1991); and Marks et al., J. Mol. Biol. 222:581-597 (1991).

The polynucleotide(s) encoding an antibody or an antigen binding fragment thereof of the disclosure can further be modified in a number of different manners using recombinant DNA technology to generate alternative antibodies. In some aspects, the constant domains of the light and heavy chains of, for example, a mouse monoclonal antibody can be substituted (1) for those regions of, for example, a human antibody to generate a chimeric antibody or (2) for a non-immunoglobulin polypeptide to generate a fusion antibody. In some aspects, the constant regions are truncated or removed to generate the desired antibody fragment of a monoclonal antibody. Site-directed or high-density mutagenesis of the variable region can be used to optimize specificity, affinity, etc. of a monoclonal antibody.

In one aspect, the antibody or antigen binding fragment thereof is a human antibody or antigen binding fragment thereof. Human antibodies can be directly prepared using various techniques known in the art. Immortalized human B lymphocytes immunized in vitro or isolated from an immunized individual that produce an antibody directed against a target antigen can be generated. See, e.g., Cole et al., Monoclonal Antibodies and Cancer Therapy, Alan R. Liss, p. 77 (1985); Boemer et al., J. Immunol. 147 (1):86-95 (1991); U.S. Pat. No. 5,750,373.

In one aspect, the antibody or antigen binding fragment thereof can be selected from a phage library, where that phage library expresses human antibodies, as described, for example, in Vaughan et al., Nat. Biotech. 14:309-314 (1996); Sheets et al., Proc. Natl. Acad. Sci. USA, 95:6157-6162 (1998); Hoogenboom and Winter, J. Mol. Biol. 227:381 (1991); and Marks et al., J. Mol. Biol. 222:581 (1991). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., J. Molec. Biol. 376:1182-1200 (2008), each of which is incorporated by reference in its entirety.

Affinity maturation strategies and chain shuffling strategies are known in the art and can be employed to generate high affinity human antibodies or antigen binding fragments thereof. See Marks et al., BioTechnology 10:779-783 (1992), incorporated by reference in its entirety.

In one aspect, the antibody or antigen binding fragment thereof (e.g. a monoclonal antibody) can be a humanized antibody. Methods for engineering, humanizing or resurfacing non-human or human antibodies can also be used and are well known in the art. A humanized, resurfaced or similarly engineered antibody can have one or more amino acid residues from a source that is non-human, e.g., but not limited to, mouse, rat, rabbit, non-human primate, or other mammal. These non-human amino acid residues are replaced by residues that are often referred to as “import” residues, which are typically taken from an “import” variable, constant or other domain of a known human sequence. Such imported sequences can be used to reduce immunogenicity or reduce, enhance or modify binding, affinity, on-rate, off-rate, avidity, specificity, half-life, or any other suitable characteristic, as known in the art. Suitably, the CDR residues may be directly and most substantially involved in influencing CD123 binding. Accordingly, part or all of the non-human or human CDR sequences are preferably maintained while the non-human sequences of the variable and constant regions can be replaced with human or other amino acids.

Antibodies can also optionally be humanized, resurfaced, engineered or human antibodies engineered with retention of high affinity for the antigen CD123 and other favourable biological properties. To achieve this goal, humanized (or human) or engineered anti-CD123 antibodies and resurfaced antibodies can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as CD123. In this way, FW residues can be selected and combined from the consensus and import sequences so that the desired antibody characteristic, such as increased affinity for the target antigen(s), is achieved.

Humanization, resurfacing or engineering of anti-CD123 antibodies or antigen binding fragments thereof of the present disclosure can be performed using any known method, such as but not limited to those described in, Jones et al., Nature 321:522 (1986); Riechmann et al., Nature 332:323 (1988); Verhoeyen et al., Science 239:1534 (1988); Sims et al., J. Immunol. 151: 2296 (1993); Chothia and Lesk, J. Mol. Biol. 196:901 (1987); Carter et al., Proc. Natl. Acad. Sci. USA 89:4285 (1992); Presta et al., J. Immunol. 151:2623 (1993); U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567, 7,557,189; 7,538,195; and 7,342,110; International Application Nos. PCT/US98/16280; PCT/US96/18978; PCT/US91/09630; PCT/US91/05939; PCT/US94/01234; PCT/GB89/01334; PCT/GB91/01134; PCT/GB92/01755; International Patent Application Publication Nos. WO90/14443; WO90/14424; WO90/14430; and European Patent Publication No. EP 229246; each of which is entirely incorporated herein by reference, including the references cited therein.

Anti-CD123 humanized antibodies and antigen binding fragments thereof can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In one aspect, a fragment (e.g. antibody fragment) of the antibody (e.g. anti-CD123 antibody) is provided. Various techniques are known for the production of antibody fragments. Traditionally, these fragments are derived via proteolytic digestion of intact antibodies, as described, for example, by Morimoto et al., J. Biochem. Biophys. Meth. 24:107-117 (1993) and Brennan et al., Science 229:81 (1985). In some aspects, anti-CD123 antibody fragments are produced recombinantly. Fab, Fv, and scFv antibody fragments can all be expressed in and secreted from E. coli or other host cells, thus allowing the production of large amounts of these fragments. Such anti-CD123 antibody fragments can also be isolated from the antibody phage libraries discussed above. The anti-CD123 antibody fragments can also be linear antibodies as described in U.S. Pat. No. 5,641,870. Other techniques for the production of antibody fragments will be apparent to the skilled practitioner.

According to the present disclosure, techniques can be adapted for the production of single-chain antibodies specific to CD123. See, e.g., U.S. Pat. No. 4,946,778). In addition, methods can be adapted for the construction of Fab expression libraries to allow rapid and effective identification of monoclonal Fab fragments with the desired specificity for CD123, or derivatives, fragments, analogs or homologs thereof. See, e.g., Huse et al., Science 246:1275-1281 (1989). Antibody fragments can be produced by techniques known in the art including, but not limited to: F(ab′)2 fragment produced by pepsin digestion of an antibody molecule; Fab fragment generated by reducing the disulfide bridges of an F(ab′)2 fragment; Fab fragment generated by the treatment of the antibody molecule with papain and a reducing agent; or Fv fragments.

In some aspects, an antibody or antigen binding fragment thereof of the disclosure can be modified in order to increase its serum half-life. This can be achieved, for example, by incorporation of a salvage receptor binding epitope into the antibody or antibody fragment, by mutation of the appropriate region in the antibody or antibody fragment or by incorporating the epitope into a peptide tag that is then fused to the antibody or antibody fragment at either end or in the middle (e.g., by DNA or peptide synthesis), or by YTE mutation. Other methods to increase the serum half-life of an antibody or antigen binding fragment thereof, e.g., conjugation to a heterologous molecule, such as PEG, are known in the art.

A modified antibody or antigen binding fragment thereof as provided herein can comprise any type of variable region that provides for the association of the antibody or polypeptide with CD123. In this regard, the variable region can comprise or be derived from any type of mammal that can be induced to mount a humoral response and generate immunoglobulins against the desired antigen. As such, the variable region of an anti-CD123 antibody or antigen binding fragment thereof can be, for example, of human, murine, non-human primate (e.g., cynomolgus monkeys, macaques, etc.) or lupine origin. In some aspects, both the variable and constant regions of the modified antibody or antigen binding fragment thereof are human. In some aspects, the variable regions of a compatible antibody (usually derived from a non-human source) can be engineered or specifically tailored to improve the binding properties or reduce the immunogenicity of the molecule. In this respect, variable regions useful in the present disclosure can be humanized or otherwise altered through the inclusion of imported amino acid sequences.

In some aspects, the variable domains in both the heavy and light chains of an antibody or antigen binding fragment thereof are altered by at least partial replacement of one or more CDRs and/or by partial framework region replacement and sequence changing. Although the CDRs can be derived from an antibody of the same class or even subclass as the antibody from which the framework regions are derived, it is envisaged that the CDRs will be derived from an antibody of different class and in certain embodiments from an antibody from a different species. It is not necessary to replace all of the CDRs with the complete CDRs from the donor variable region to transfer the antigen binding capacity of one variable domain to another. Rather, it is only necessary to transfer those residues that are necessary to maintain the activity of the antigen binding site. Given the explanations set forth in U.S. Pat. Nos. 5,585,089, 5,693,761 and 5,693,762, it will be well within the competence of those skilled in the art to carry out routine experimentation to obtain a functional antibody with reduced immunogenicity.

Alterations to the variable region notwithstanding, those skilled in the art will appreciate that a modified antibody or antigen binding fragment thereof of this disclosure will comprise an antibody (e.g., full-length antibody or antigen binding fragment thereof) in which at least a fraction of one or more of the constant region domains has been deleted or otherwise altered so as to provide desired biochemical characteristics such as increased tumour localization or reduced serum half-life when compared with an antibody of approximately the same immunogenicity comprising a native or unaltered constant region. In some aspects, the constant region of the modified antibody will comprise a human constant region. Modifications to the constant region compatible with this disclosure comprise additions, deletions or substitutions of one or more amino acids in one or more domains. That is, the modified antibody disclosed herein can comprise alterations or modifications to one or more of the three heavy chain constant domains (CH1, CH2 or CH3) and/or to the light chain constant domain (CL). In some aspects, a modified constant region wherein one or more domains are partially or entirely deleted are contemplated. In some aspects, a modified antibody will comprise domain deleted constructs or variants wherein the entire CH2 domain has been removed (ΔCH2 constructs). In some aspects, the omitted constant region domain can be replaced by a short amino acid spacer (e.g., 10 residues) that provides some of the molecular flexibility typically imparted by the absent constant region.

Besides their configuration, it is known in the art that the constant region mediates several effector functions. For example, antibodies bind to cells via the Fc region, with an Fc receptor site on the antibody Fc region binding to an Fc receptor (FcR) on a cell. There are a number of Fc receptors that are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In some aspects, an antibody or an antigen binding fragment thereof provides for altered effector functions that, in turn, affect the biological profile of the administered antibody or antigen binding fragment thereof. For example, the deletion or inactivation (through point mutations or other means) of a constant region domain can reduce Fc receptor binding of the circulating modified antibody. In other cases it can be that constant region modifications, consistent with this disclosure, moderate complement binding and thus reduce the serum half-life and nonspecific association of a conjugated cytotoxin. Yet other modifications of the constant region can be used to eliminate disulfide linkages or oligosaccharide moieties that allow for enhanced localization due to increased antigen specificity or antibody flexibility. Similarly, modifications to the constant region in accordance with this disclosure can easily be made using well-known biochemical or molecular engineering techniques well within the purview of the skilled artisan.

In some aspects, the antibody or antigen binding fragment thereof does not have one or more effector functions. For instance, in some aspects, the antibody or antigen binding fragment thereof has no antibody-dependent cellular cytoxicity (ADCC) activity and/or no complement-dependent cytoxicity (CDC) activity. In some aspects, the antibody or antigen binding fragment thereof does not bind to an Fc receptor and/or complement factors. In some aspects, the antibody or antigen binding fragment thereof has no effector function.

In some aspects, the antibody or antigen binding fragment thereof can be engineered to fuse the CH3 domain directly to the hinge region of the respective modified antibodies or fragments thereof. In other constructs a peptide spacer can be inserted between the hinge region and the modified CH2 and/or CH3 domains. For example, compatible constructs can be expressed in which the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. Amino acid spacers can, in some cases, prove to be immunogenic and elicit an unwanted immune response against the construct. In some aspects, any spacer added to the construct can be relatively non-immunogenic, or even omitted altogether, so as to maintain the desired biochemical qualities of the modified antibodies.

Besides the deletion of whole constant region domains, an antibody or antigen binding fragment thereof provided herein can be modified by the partial deletion or substitution of a few or even a single amino acid in a constant region. For example, the mutation of a single amino acid in selected areas of the CH2 domain can be enough to substantially reduce Fc binding and thereby increase tumor localization. Similarly one or more constant region domains that control the effector function (e.g., complement C1Q binding) can be fully or partially deleted. Such partial deletions of the constant regions can improve selected characteristics of the antibody or antigen binding fragment thereof (e.g., serum half-life) while leaving other desirable functions associated with the subject constant region domain intact. Moreover, the constant regions of the antibody and antigen binding fragment thereof can be modified through the mutation or substitution of one or more amino acids that enhances the profile of the resulting construct. In this respect it is possible to disrupt the activity provided by a conserved binding site (e.g., Fc binding) while substantially maintaining the configuration and immunogenic profile of the modified antibody or antigen binding fragment thereof. In some aspects, there may be an addition of one or more amino acids to the constant region to enhance desirable characteristics such as decreasing or increasing effector function or provide for more cytotoxin or carbohydrate attachment. In some aspects, it can be desirable to insert or replicate specific sequences derived from selected constant region domains.

The present disclosure further embraces variants and equivalents that are substantially homologous an antibody or antigen binding fragment of the disclosure (e.g. murine, chimeric, humanized or human antibody, or antigen binding fragments thereof). These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art.

In some aspects, the antibody or antigen binding fragment thereof can be further modified to contain additional chemical moieties not normally part of the protein. Those derivatized moieties can improve the solubility, the biological half-life or absorption of the protein. The moieties can also reduce or eliminate any desirable side effects of the proteins and the like. An overview for those moieties can be found in Remington's Pharmaceutical Sciences, 22nd ed., Ed. Lloyd V. Allen, Jr. (2012).

### III. Polynucleotides

Provided are polynucleotides comprising a nucleotide sequence encoding an antibody or antigen binding fragment thereof described herein. In some aspects, the polynucleotides include sequences that have been removed from their naturally occurring environment, recombinant or cloned (e.g. DNA) isolates, and chemically synthesized analogues or analogues biologically synthesized by heterologous systems.

In some aspects, the polynucleotide sequence(s) are prepared by any means known in the art. For example, large amounts of the polynucleotide sequence(s) may be produced by replication and/or expression in a suitable host cell. In some aspects, natural or synthetic DNA fragments coding for an antibody or antigen binding fragment described herein are incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured bacterial, insect, mammalian, plant or other eukaryotic cell line.

In some aspects, the polynucleotide sequences are prepared by chemical synthesis, e.g. by the phosphoramidite method or the tri-ester method and may be performed on commercial automated oligonucleotide synthesizers. A double-stranded (e.g. DNA) fragment may be obtained from a single stranded product of chemical synthesis either by synthesizing the complementary strand and annealing the strand together under appropriate conditions or by adding the complementary strand using DNA polymerase with an appropriate primer sequence.

In some aspects, the polynucleotide sequences are “isolated,” which denotes that the sequences have been removed from their natural genetic milieu and are thus free of other extraneous or unwanted coding sequences (but may include naturally occurring 5′ and 3′ untranslated regions such as promoters and terminators), and are in a form suitable for use within genetically engineered protein production systems. Such isolated polynucleotides are those that are separated from their natural environment.

In some aspects, variants of polynucleotides described above are provided. In some aspects, polynucleotide variants contain alterations in the coding regions, non-coding regions, or both. In some aspects, polynucleotide variants comprise alterations that produce silent substitutions, additions, or deletions, but do not alter the properties or activities of the encoded polypeptide. In some aspects, polynucleotide variants are produced by a silent substitution due to the degeneracy of the genetic code. In some aspects, polynucleotide variants are produced for a variety of reasons, e.g., to optimize codon expression for a particular host (change codons, e.g., in a mammalian sequence to those preferred by a prokaryotic cell).

In some aspects, the polynucleotides comprise a nucleotide sequence encoding an antibody or antigen binding fragment thereof that comprises an antigen binding domain comprising a VH-CDR1 comprising the amino acid sequence set forth in SEQ ID NO: 1, a VH-CDR2 comprising the amino acid sequence set forth in SEQ ID NO: 2, a VH-CDR3 comprising the amino acid sequence set forth in SEQ ID NO: 3, a VL-CDR1 comprising the amino acid sequence set forth in SEQ ID NO: 4, a VL-CDR2 comprising the amino acid sequence set forth in SEQ ID NO: 5, and a VL-CDR3 comprising the amino acid sequence set forth in SEQ ID NO: 6.

In some aspects, the polynucleotides comprise a nucleotide sequence encoding an antibody or antigen binding fragment thereof that comprises an antigen binding domain comprising a VH-CDR1 comprising the amino acid sequence set forth in SEQ ID NO: 9, a VH-CDR2 comprising the amino acid sequence set forth in SEQ ID NO: 10, a VH-CDR3 comprising the amino acid sequence set forth in SEQ ID NO: 11, a VL-CDR1 comprising the amino acid sequence set forth in SEQ ID NO: 12, a VL-CDR2 comprising the amino acid sequence set forth in SEQ ID NO: 13, and a VL-CDR3 comprising the amino acid sequence set forth in SEQ ID NO: 14.

In some aspects, the polynucleotide comprises a nucleotide sequence encoding an antibody or antigen binding fragment thereof comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the polynucleotide comprises a nucleotide sequence encoding an antibody or antigen binding fragment thereof comprising a VH comprising an amino acid sequence as set forth in SEQ ID NO: 7, and a VL comprising an amino acid sequences as set forth in SEQ ID NO: 8.

In some aspects, the polynucleotide comprises a nucleotide sequence encoding an antibody or antigen binding fragment thereof comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 15, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16. In some aspects, the polynucleotide comprises a nucleotide sequence encoding an antibody or antigen binding fragment thereof comprising a VH comprising an amino acid sequence as set forth in SEQ ID NO: 15, and a VL comprising an amino acid sequences as set forth in SEQ ID NO: 16.

In some aspects, the polynucleotide is comprised in a cell.

In some aspects, the cell comprises a polynucleotide of SEQ ID NO: 118.

In some aspects, the polynucleotides of SEQ ID NO: 118 and SEQ ID NO: 119 are present on separate polynucleotides. In some aspects, the polynucleotides of SEQ ID NO: 118 and SEQ ID NO: 119 are present on the same polynucleotide.

In some aspects, the polynucleotides as described herein are present in a vector. As such, provided herein are vectors comprising a polynucleotide of the present disclosure. In some aspects, provided is a vector or a set of vectors comprising polynucleotides encoding antibodies or antigen binding fragments thereof as described herein. In some aspects, provided is a vector or a set of vectors comprising polynucleotides encoding antibodies or antigen binding fragments thereof that bind to CD123 as disclosed herein.

In some aspects, the polynucleotides as described herein comprise regulatory elements that initiate expression of an antibody or antigen binding fragment thereof. In some aspects, the polynucleotides comprise a promoter operably linked to a nucleic acid that encodes an antibody or antigen binding fragment thereof. In some aspects, the polynucleotides comprise a first promoter operably linked to a first nucleic acid that encodes a first polypeptide of an antibody or antigen binding fragment thereof. In some aspects, the polynucleotides comprise a second promoter operably linked to a second nucleic acid that encodes a second polypeptide of an antibody or antigen binding fragment thereof.

In some aspects, the first and the second promoter are the same. In some aspects, the first and the second promoter are different promoters. In some aspects, the promoters are inducible promoters. In some aspects, the promoters are constitutive promoters.

In some aspects, the polynucleotides further comprise polyadenylation signal sequences. In some aspects, the polyadenylation signal sequence is an SV40 polyadenylation signal sequence, a human growth hormone polyadenylation signal sequence, or a bovine growth hormone polyadenylation signal sequence.

In some aspects, the polynucleotides further comprise an IRES.

In some aspects, the polynucleotides comprise in 5′ to 3′ direction a first nucleic acid that encodes a first polypeptide of an antibody or antigen binding fragment thereof, an IRES, and a second nucleic acid that encodes a second polypeptide of an antibody or antigen binding fragment thereof.

In some aspects, the set of vectors comprises a first vector and a second vector, wherein the first vector comprises a nucleic acid sequence encoding a polypeptide chain of an antibody or antigen binding fragment described herein, and the second vector comprises a nucleic acid sequence encoding a second polypeptide chain of an antibody or antigen binding fragment described herein. For example, a first vector comprises a nucleic acid sequence encoding a heavy chain of an antibody or antigen binding fragment described herein, and the second vector comprises a nucleic acid sequence encoding a light chain of an antibody or antigen binding fragment described herein or vice versa.

Any vector known in the art is suitable for the present disclosure. In some aspects, the vector is a viral vector. In some aspects, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof.

In some aspects, the polynucleotide is present in a cell. In some aspects, a cell comprises a polynucleotide or a vector described herein. In some aspects, a cell comprises a polynucleotide encoding an antibody or antigen binding fragment thereof described herein. In some aspects, a cell comprises a vector comprising a polynucleotide encoding an antibody or an antigen binding fragment thereof as described herein. In some aspects, the cell comprises a vector comprising a polynucleotide encoding an antibody or an antigen binding fragment thereof that specifically binds to CD123 as described herein. In some aspects, the cell comprising a polypeptide as described herein is used to produce an antibody or an antigen binding fragment thereof that specifically binds to CD123.

Any cell may be used as a host cell for the polynucleotides, the vectors, or the polypeptides of the present disclosure. In some aspects, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. aeruginosa; and Streptomyces. In some aspects, the cell is a human cell.

TABLE 1
Sequences

| SEQ | | |
|---|---|---|
| ID | | |
| NO. | Clone | Sequence |
| 1 | HT12-GL | IYAMT |
| | HCDR1 | |
| 2 | HT12-GL | GISAGGDSTYYADSVKG |
| | HCDR2 | |
| 3 | HT12-GL | DRGYYYGMDV |
| | HCDR3 | |
| 4 | HT12-GL | RSRQSLLRSNGYNYLD |
| | LCDR1 | |
| 5 | HT12-GL | LGSNRAS |
| | LCDR2 | |
| 6 | HT12-GL | MQALQTPYT |
| | LCDR3 | |
| 7 | HT12-GL | EVQLLESGGGLVQPGGSLRLSCAASGFTFNIYAMTWVRQAPGKGLEWVSGISAGGDS |
| | VH | TYYADSVKGRFTISRDNSKNTLYLQMNSLRAEDTAVYYCAKDRGYYYGMDVWGQGTT |
| | | VTVSS |
| 8 | HT12-GL | DIVMTQSPLSLPVTPGEPASISCRSRQSLLRSNGYNYLDWYLQKPGQSPQLLIYLGS |
| | VL | NRASGVPDRFSGSGSGTDFTLKISRVEAEDVGVYYCMQALQTPYTFGQGTKLEIK |
| 9 | J13 HCDR1 | SYAMS |
| 10 | J13 HCDR2 | AISGSGDSTYYADSVKG |
| 11 | J13 HCDR3 | DRGFYYGMDV |
| 12 | J13 LCDR1 | RSSQSLLRSNGYNYLD |
| 13 | J13 LCDR2 | LGSNRAS |
| 14 | J13 LCDR3 | MQALQTPFT |
| 15 | J13 VH | EVQLVESGGGLVQPGGSLRLSCVVSGFTFSSYAMSWVRQAPGKGLEWVSAISGSGDS |
| | | TYYADSVKGRFTFSRDNSKNTLYLQMNSLRAEDTAVYYCTKDRGFYYGMDVWGQGTM |
| | | VTVSS |
| 16 | J13 VL | DVVMTQSPLSLPVTPGEPASISCRSSQSLLRSNGYNYLDWYLQKPGQSPQLLIYLGS |
| | | NRASGVPDRFSGSGSGTDFTLKISRVEAEDVGVYYCMQALQTPFTFGQGTRLEIK |
| 17 | 008QH7-J13 | DVVMTQSPLSLPVTPGEPASISCRSSQSLLRSNGYNYLDWYLQKPGQSPQLLIYLGS |
| | VK | NRASGVPDRFSGSGSGTDFTLKISRVEAEDVGVYYCMQALQTPFTFGQGTRLEIKR |
| 18 | 008QH7-J13 | EVQLVESGGGLVQPGGSLRLSCAASGFTFSSYAMSWVRQAPGKGLEWVSAISGSGDS |
| | VH Germline | TYYADSVKGRFTFSRDNSKNTLYLQMNSLRAEDTAVYYCTKDRGFYYGMDVWGQGTT |
| | | VTVSS |
| 19 | 008QH7-J13 | DVVMTQSPLSLPVTPGEPASISCRSSQSLLRSNGYNYLDWYLQKPGQSPQLLIYLGS |
| | VL Germline | NRASGVPDRFSGSGSGTDFTLKISRVEAEDVGVYYCMQALQTPFTFGQGTRLEIKR |
| 64 | 008QH7- | QVQLQESGPGLVKPSETLSLTCTVSGGSISSYYWSWIRQPAGKGLEWIGRIYTSEST |
| | B21_VH | NYNPSLKSRVTMSVDTSKNQFSLKLNSVTAADTAVYYCARAPYGGLDYWGQGTLVTV |
| | | SS |
| 65 | 008QH7- | DIQMTQSPSSLSASVGDRVTITCRASQGIRNDLGWYQQKPGKVPKLLIYAASSLQSG |
| | B21_VK | VPSRFSGSGSGTDFTLTISSLQPEDFATYYCLQDYNYPYTFGPGTKVDIK |
| 67 | HT1-2108R- | DIVMTQSPLSLPVTPGEPASISCRSSQSLLHSNGYNHLDWYLQKPGQSPQLLIYLGS |
| | 02-VK_GL | NRASGVPDRFSGSGSGTDFTLKISRVEAEDVGVYYCMQALQTLTWTFGQGTKVEIK |
| 68 | HT4-2108R- | EVQLLESGGGLVQPGGSLRLSCAASGFTFSIYAMSWVRQAPGKGLEWVSAISGGGDS |
| | 04-VH_GL | TYNADSVKGRFTISRDNSKNTLYLQMNSLRAEDTAVYYCAKDRGYYYGMVVWGQGTT |
| | | VTVSS |
| 69 | HT4-2108R- | DIVMTQSPLSLPVTPGEPASISCRSSQSLLRSNGYNYLDWYLQKPGQSPQLLIYLGS |
| | 38-VK_GL | NRASGVPDRFSGSGSGTDFTLKISRVEAEDVGVYYCMQALQTPYTFGQGTKLEIK |
| 70 | HT6-2108R- | EVQLLESGGGLVQPGGSLRLSCAASGFTENSYAMSWVRQAPGKGLEWVSAVSGGGDT |
| | 06-VH_GL | TYYAESVKGRFTISRDNSKNTLYLQMNSLRAEDTAVYYCAKDRGYYYGMAVWGQGTT |
| | | VTVSS |
| 71 | HT6-2108R- | DIVMTQSPLSLPVTPGEPASISCRSSQSLLRSNGYHYLDWYLQKPGQSPQLLIYLGS |
| | 05-VK_GL | NRAPGVPDRFSGSGSGTDFTLKISRVEAEDVGVYYCMQALQTPYTFGQGTKLEIK |
| 72 | HT7-2108R- | EVQLLESGGGLVQPGGSLRLSCAASGFTFSHYAMSWVRQAPGKGLEWVSAISGGGDR |
| | 07-VH_GL | TFYADSVKGRFTISRDNSKNTLYLQMNSLRAEDTAVYYCAKDRGFYYGMDVWGQGTT |
| | | VTVSS |
| 73 | HT7-2108R- | DIVMTQSPLSLPVTPGEPASISCRSSQSLLHSNGYNYLDWYLQKPGQSPQLLIYLGS |
| | 35-VK_GL | NRASGVPDRFSGSGSGTDFTLKISRVEAEDVGVYYCMQALQTPYTFGQGTKLEIK |
| 74 | HT8-2108R- | EVQLLESGGGLVQPGGSLRLSCAASGFTFSIYAMSWVRQAPGKGLEWVSAISGGGDT |
| | 08-VH_GL | TYYADSMKGRFTISRDNSKNTLYLQMNSLRAEDTAVYYCAKDRGYYYGMDVWGQGTT |
| | | VTVSS |
| 75 | HT8-2108R-35- | DIVMTQSPLSLPVTPGEPASISCRSSQSLLHSNGYNYLDWYLQKPGQSPQLLIYLGS |
| | VK GL | NRASGVPDRFSGSGSGTDFTLKISRVEAEDVGVYYCMQALQTPYTFGQGTKLEIK |
| 76 | HT10-2108R-10- | EVQLLESGGGLVQPGGSLRLSCAASGFTFSHYAMTWVRQAPGKGLEWISAISGGGDS |
| | VH_GL | TYYADSVKGRFTISRDNSKNTLYLQMNSLRAEDTAVYYCAKDRGYYYGMDDWGQGTT |
| | | VTVSS |
| 77 | HT10-2108R-35- | DIVMTQSPLSLPVTPGEPASISCRSSQSLLHSNGYNYLDWYLQKPGQSPQLLIYLGS |
| | VK_GL | NRASGVPDRFSGSGSGTDFTLKISRVEAEDVGVYYCMQALQTPYTFGQGTKLEIK |
| 79 | HT11-2108R-11- | DIQMTQSPSSLSASVGDRVTITCRASQGINNYLAWYQQKPGKVPKLLIYAASTLHSG |
| | VK GL | VPSRFSGSGSATDFTLTISSLQPEDVATYYCQKYNSAPFTFGPGTKVDIK |
| 80 | HT14-2108R-14- | EVQLLESGGGLVQPGGSLRLSCAASGFTFNIYAMTWVRQAPGKGLEWVSGISGGGDS |
| | VH_GL | TYYADSVKGRFTMSRDNSKNTLYLQMNSLRAEDTAVYYCAKDRGYYYGMDVWGQGTT |
| | | VTVSS |
| 81 | HT14-2108R-14- | DIVMTQSPLSLPVTPGEPASISCKSRQSLLRSNGYNYLDWYLQKPGQSPQLLIYLGS |
| | VK GL | NRASGVPDRFSGSGSGTDFTLKISRVEAEDVGVYYCMQALQTPYTFGQGTKLEIK |
| 82 | HT16-2108R-16- | EVQLLESGGGLVQPGGSLRLSCAASGFTFSNYAMTWVRQAPGKGLEWVSAISGGGDT |
| | VH_GL | TYYADSVKGRFTISRDNSKNTLYLQMNSLRAEDTAVYYCAKDRGYYYGMDVWGQGTT |
| | | VTVSS |
| 83 | HT16-2108R-38- | DIVMTQSPLSLPVTPGEPASISCRSSQSLLRSNGYNYLDWYLQKPGQSPQLLIYLGS |
| | VK_GL | NRASGVPDRFSGSGSGTDFTLKISRVEAEDVGVYYCMQALQTPYTFGQGTKLEIK |
| 85 | HT22-2108R-20- | EIVLTQSPATLSSSPGERATLSCRASQSVSSYLAWYQQKPGQVPRLLIYDASNRATG |
| | VK_GL-H | IPARFSGSGSGTDFTLTISNLEPEDFAVYYCQQRSSWPLTFGGGTEVEIK |
| 87 | HT22-2108R-20- | EIVLTQSPATLSLSPGERATLSCRASQSVSSYLAWYQQKPGQAPRLLIYDASNRATG |
| | VK_GL | IPARFSGSGSGTDFTLTISSLEPEDFAVYYCQQRSSWPLTFGGGTEVEIK |
| 88 | HT28-2108R-28- | EVQLLESGGGLVQPGGSLRLSCAASGFTFSSYAMTWVRQAPGKGLEWVSAISGGGDT |
| | VH_GL | TYCADSVKGRFTISRDNSKNTLYLQMNSLRAEDTAVYYYAKDRGYYYGMDVWGQGTT |
| | | VTVSS |
| 89 | HT28-2108R-38- | DIVMTQSPLSLPVTPGEPASISCRSSQSLLRSNGYNYLDWYLQKPGQSPQLLIYLGS |
| | VK_GL | NRASGVPDRFSGSGSGTDFTLKISRVEAEDVGVYYCMQALQTPYTFGQGTKLEIK |
| 90 | HT29-2108R-29- | EVQLLESGGGLVQPGGSLRLSCAASGFAFSHYAMSWVRQAPGKGLEWVSAISGGGDS |
| | VH_GL | TYYADSVKGRFTISRDNSKNTLYLQMNSLRAEDTAVYYCAKDRGYYYGMDVWGQGTT |
| | | VTVSS |
| 91 | HT29-2108R-38- | DIVMTQSPLSLPVTPGEPASISCRSSQSLLRSNGYNYLDWYLQKPGQSPQLLIYLGS |
| | VK_GL | NRASGVPDRFSGSGSGTDFTLKISRVEAEDVGVYYCMQALQTPYTFGQGTKLEIK |
| 92 | HT30-2108R-30- | EVQLLESGGGLVQPGGSLRLSCAASGFTFSIYAMSWVRQAPGKGLEWVSAITAGGDT |
| | VH_GL | TYYAVSVKGRFTISRDNSKNTLYLQMNSLRAEDTAVYYCAKDRGYYYGMDVWGQGTT |
| | | VTVSS |
| 93 | HT30-2108R-35- | DIVMTQSPLSLPVTPGEPASISCRSSQSLLHSNGYNYLDWYLQKPGQSPQLLIYLGS |
| | VK_GL | NRASGVPDRFSGSGSGTDFTLKISRVEAEDVGVYYCMQALQTPYTFGQGTKLEIK |
| 94 | HT31-2108R-31- | EVQLLESGGGLVQPGGSLRLSCAASGFTFSIYAMSWVRQAPGKGLEWVSAISGGGDS |
| | VH_GL | TYYADSVKGRFTISRDNSKNTLYLQMNSLRAEDTAVYYCAKDRGFYYGMDVWGQGTT |
| | | VTVSS |
| 95 | HT31-2108R-38- | DIVMTQSPLSLPVTPGEPASISCRSSQSLLRSNGYNYLDWYLQKPGQSPQLLIYLGS |
| | VK_GL | NRASGVPDRFSGSGSGTDFTLKISRVEAEDVGVYYCMQALQTPYTFGQGTKLEIK |
| 96 | HT32-2108R-32- | EVQLLESGGGLVQPGGSLRLSCAASGFTFSSYAMSWVRQAPGKGLEWVSAISGGGDT |
| | VH_GL | TYYADSVKGRFTISRDNSKNTLYLQMNSLRAEDTAVYYCAKDRGFYYGMDVWGQGTT |
| | | VTVSS |
| 97 | HT32-2108R-38- | DIVMTQSPLSLPVTPGEPASISCRSSQSLLRSNGYNYLDWYLQKPGQSPQLLIYLGS |
| | VK GL | NRASGVPDRFSGSGSGTDFTLKISRVEAEDVGVYYCMQALQTPYTFGQGTKLEIK |
| 98 | HT33-2108R-33- | EVQLLESGGGLVQPGGSLRLSCAASGFTFGHYAMSWVRQAPGKGLEWVSAISGGGDY |
| | VH_GL | TYYADSVKGRLTISRDNSKNTLYLQMNSLRAEDTAVYYCAKDRGYYYGMDVWGQGTT |
| | | VTVSS |
| 99 | HT33-2108R-33- | DIVMTQSPLSLPVTPGEPASISCRSSQSLLHSNGYKYLDWYLQKPGQSPQLLIYLGS |
| | VK_GL | NRASGVPDRFSGSGSGTDFTLKISRVEAEDVGVYYCMQALQIPYTFGQGTKLEIK |
| 100 | HT34-2108R-34- | EVQLLESGGGLVQPGGSLRLSCAASGFTFSHYAMTWVRQAPGKGLEWVSAISGGGDT |
| | VH_GL-L | TYYADSVKGRFTISRDNSKTTLYLQMNSLRAEDTAVYYCAKDRGYYYGMDVWGQGTT |
| | | VTVSS |
| 101 | HT34-2108R-34- | DIVMTQSPLSLPVTPGEPASISCRSGQSLLRSNGYNYLDWYLQKPGQSPQLLIYLGS |
| | VK GL-L | NRASGVPDRFSGSGSGTDFTLKISRVEAEDVGVYYCMQALQTPFTFGPGTKVDIK |
| 102 | HT34-2108R-34- | EVQLLESGGGLVQPGGSLRLSCAASGFTFSHYAMTWVRQAPGKGLEWVSAISGGGDT |
| | VH_GL | TYYADSVKGRFTISRDNSKNTLYLQMNSLRAEDTAVYYCAKDRGYYYGMDVWGQGTT |
| | | VTVSS |
| 103 | HT34-2108R-34- | DIVMTQSPLSLPVTPGEPASISCRSGQSLLRSNGYNYLDWYLQKPGQSPQLLIYLGS |
| | VK_GL | NRASGVPDRESGSGSGTDFTLKISRVEAEDVGVYYCMQALQTPFTFGPGTKVDIK |
| 104 | HT35-2108R-35- | EVQLLESGGGLVQPGGSLRLSCAASGFTFSHYAMTWVRQAPGKGLEWISAISGGGDS |
| | VH_GL | TYYADSVKGRFTISRDNSKNTLYLQMNSLRAEDTAVYYCAKDRGYYYGMDVWGQGTT |
| | | VTVSS |
| 105 | HT35-2108R-35- | DIVMTQSPLSLPVTPGEPASISCRSSQSLLHSNGYNYLDWYLQKPGQSPQLLIYLGS |
| | VK_GL | NRASGVPDRFSGSGSGTDFTLKISRVEAEDVGVYYCMQALQTPYTFGQGTKLEIK |
| 106 | HT36-2108R-36- | EVQLLESGGGLVQPGGSLRLSCAASGFTFSIYAMSWVRQAPGKGLEWVSAISGGGDT |
| | VH_GL | TYYADSVKGRFTSSRDNSKNTLYLQMNSLRAEDTAVYFCAKDRGFYYGMDVWGQGTT |
| | | VTVSS |
| 107 | HT36-2108R-36- | DIVMTQSPLSLPVTPGEPASISCRSSQSLLRSNGYIYLDWYLQKPGQSPQLLIYLGS |
| | VK GL | NRASGVPDRFSGSGSGTDFTLKISRVEAEDVGVYYCMQALQTPYTFGQGTKLEIK |
| 108 | HT37-2108R-37- | EVQLLESGGGLVQPGGSLRLSCAASGFTFSIYAMTWVRQAPGKGLEWVSAISAGGDN |
| | VH_GL-L | TYNADSVKGRFTISRDNSKITLYLQMNSLRAEDTAVYYCAKDRGYYYGMDVWGQGTT |
| | | VTVSS |
| 109 | HT37-2108R-38- | DIVMTQSPLSLPVTPGEPASISCRSSQSLLRSNGYNYLDWYLQKPGQSPQLLIYLGS |
| | VK GL-L | NRASGVPDRFSGSGSGTDFTLKISRVEAEDVGVYYCMQALQTPYTFGQGTKLEIK |
| 110 | HT37-2108R-37- | EVQLLESGGGLVQPGGSLRLSCAASGFTFSIYAMTWVRQAPGKGLEWVSAISAGGDN |
| | VH_GL | TYNADSVKGRFTISRDNSKNTLYLQMNSLRAEDTAVYYCAKDRGYYYGMDVWGQGTT |
| | | VTVSS |
| 111 | HT37-2108R-38- | DIVMTQSPLSLPVTPGEPASISCRSSQSLLRSNGYNYLDWYLQKPGQSPQLLIYLGS |
| | VK_GL | NRASGVPDRFSGSGSGTDFTLKISRVEAEDVGVYYCMQALQTPYTFGQGTKLEIK |
| 112 | HT38-2108R-38- | EVQLLESGGGLVQPGGSLRLSCAASGFTFSSYAMSWVRQAPGKGLEWVSAISGGGDS |
| | VH_GL | TYYTDSVKGRFTISRDNSKNTLYLQMNSLRAEDTAVYYCAKDRGFYYGMDVWGQGTT |
| | | VTVSS |
| 113 | HT38-2108R-38- | DIVMTQSPLSLPVTPGEPASISCRSSQSLLRSNGYNYLDWYLQKPGQSPQLLIYLGS |
| | VK_GL | NRASGVPDRFSGSGSGTDFTLKISRVEAEDVGVYYCMQALQTPYTFGQGTKLEIK |
| 114 | HT38-2108R-38- | EVQLLESGGGLVQPGGSLRLSCAASGFTFSSYAMSWVRQAPGKGLEWVSAISGGGDS |
| | VH_GL-Q | TYYTDSVKGRFTISRDNSKNTLYLQMNSLRAEDTAVYYCAKDRGFYYGMDVWGQGTT |
| | | VTVSS |
| 115 | HT38-2108R-38- | DIVMTQSPLSLPVTPGEPASISCRSSQSLLRSQGYNYLDWYLQKPGQSPQLLIYLGS |
| | VK_GL-Q | NRASGVPDRFSGSGSGTDFTLKISRVEAEDVGVYYCMQALQTPYTFGQGTKLEIK |
| 116 | HT12-GL Heavy | EVQLLESGGGLVQPGGSLRLSCAASGFTFNIYAMTWVRQAPGKGLEWVSGISAGGDS |
| | Chain | TYYADSVKGRFTISRDNSKNTLYLQMNSLRAEDTAVYYCAKDRGYYYGMDVWGQGTT |
| | (CDRs in | VTVSSASTKGPSVFPLAPSSKSTSGGTAALGCLVKDYFPEPVTVSWNSGALTSGVHT |
| | shown in bold | FPAVLQSSGLYSLSSVVTVPSSSLGTQTYICNVNHKPSNTKVDKRVEPKSCDKTHTC |
| | according | PPCPAPELLGGPSVFLFPPKPKDTLMISRTPEVTCVVVDVSHEDPEVKENWYVDGVE |
| | to Kabat) | VHNAKTKPREEQYNSTYRVVSVLTVLHQDWLNGKEYKCKVSNKALPAPIEKTISKAK |
| | (conjugation | GQPREPQVYTLPPSREEMTKNQVSLTCLVKGFYPSDIAVEWESNGQPENNYKTTPPV |
| | sites | LDSDGSFFLYSKLTVDKSRWQQGNVFSCSVMHEALHNHYTQKSLSLSPGK |
| | underlined) | |
| 117 | HT12-GL Light | DIVMTQSPLSLPVTPGEPASISCRSRQSLLRSNGYNYLDWYLQKPGQSPQLLIYLGS |
| | Chain | NRASGVPDRFSGSGSGTDFTLKISRVEAEDVGVYYCMQALQTPYTFGQGTKLEIKRT |
| | (CDRs in | VAAPSVFIFPPSDEQLKSGTASVVCLLNNFYPREAKVQWKVDNALQSGNSQESVTEQ |
| | shown in bold | DSKDSTYSLSSTLTLSKADYEKHKVYACEVTHQGLSSPVTKSENRGEC |
| | according | |
| | to Kabat) | |
| | (conjugation | |
| | site | |
| | underlined) | |
| 118 | HT12-GL | GAGGTTCAGCTTCTGGAATCTGGGGGCGGTCTTGTACAGCCGGGGGGCTCCCTTCGC |
| | Heavy Chain | CTGTCTTGTGCCGCATCTGGGTTCACGTTTAATATCTATGCAATGACGTGGGTGCGA |
| | nucleotides | CAAGCGCCTGGGAAAGGATTGGAGTGGGTGTCAGGGATTTCTGCTGGAGGTGATTCA |
| | | ACATACTACGCGGATAGTGTCAAAGGCAGATTCACGATAAGCCGGGATAATTCAAAA |
| | | AACACGCTTTACCTGCAAATGAACTCCTTGCGGGCTGAAGACACAGCAGTCTATTAC |
| | | TGTGCGAAGGATCGGGGCTACTACTACGGGATGGATGTTTGGGGCCAGGGGACAACC |
| | | GTCACGGTCTCATCCGCGTCGACCAAGGGCCCATCCGTCTTCCCCCTGGCACCCTCC |
| | | TCCAAGAGCACCTCTGGGGGCACAGCGGCCCTGGGCTGCCTGGTCAAGGACTACTTC |
| | | CCCGAACCGGTGACGGTGTCCTGGAACTCAGGCGCTCTGACCAGCGGCGTGCACACC |
| | | TTCCCGGCTGTCCTACAGTCCTCAGGACTCTACTCCCTCAGCAGCGTGGTGACCGTG |
| | | CCCTCCAGCAGCTTGGGCACCCAGACCTACATCTGCAACGTGAATCACAAGCCCAGC |
| | | AACACCAAGGTGGACAAGAGAGTTGAGCCCAAATCTTGTGACAAAACTCACACATGC |
| | | CCACCGTGCCCAGCACCTGAACTCCTGGGGGGACCGTCAGTCTTCCTCTTCCCCCCA |
| | | AAACCCAAGGACACCCTCATGATCTCCCGGACCCCTGAGGTCACATGCGTGGTGGTG |
| | | GACGTGAGCCACGAAGACCCTGAGGTCAAGTTCAACTGGTACGTGGACGGCGTGGAG |
| | | GTGCATAATGCCAAGACAAAGCCGCGGGAGGAGCAGTACAACAGCACGTACCGTGTG |
| | | GTCAGCGTCCTCACCGTCCTGCACCAGGACTGGCTGAATGGCAAGGAGTACAAGTGC |
| | | AAGGTCTCCAACAAAGCCCTCCCAGCCCCCATCGAGAAAACCATCTCCAAAGCCAAA |
| | | GGGCAGCCCCGAGAACCACAGGTCTACACCCTGCCCCCATCCCGGGAGGAGATGACC |
| | | AAGAACCAGGTCAGCCTGACCTGCCTGGTCAAAGGCTTCTATCCCAGCGACATCGCC |
| | | GTGGAGTGGGAGAGCAATGGGCAGCCGGAGAACAACTACAAGACCACGCCTCCCGTG |
| | | CTGGACTCCGACGGCTCCTTCTTCCTCTATAGCAAGCTCACCGTGGACAAGAGCAGG |
| | | TGGCAGCAGGGGAACGTCTTCTCATGCTCCGTGATGCATGAGGCTCTGCACAACCAC |
| | | TACACGCAGAAGAGCCTCTCCCTGTCTCCGGGTAAA |
| 119 | HT12-GL Light | GACATCGTTATGACCCAAAGTCCTCTCTCACTCCCCGTTACCCCTGGAGAACCAGCA |
| | Chain | AGCATTAGTTGCAGGAGCCGACAGAGCCTGTTGCGGTCTAATGGCTATAATTATCTG |
| | nucleotides | GATTGGTATCTGCAGAAACCTGGTCAATCACCACAGCTTCTCATATATCTGGGAAGC |
| | | AACCGCGCTTCCGGTGTGCCAGACCGCTTCTCAGGATCTGGTTCTGGCACAGATTTC |
| | | ACGTTGAAAATCAGCCGAGTTGAAGCGGAGGATGTGGGTGTCTATTACTGCATGCAA |
| | | GCACTGCAGACACCATATACGTTCGGCCAAGGAACAAAACTGGAAATTAAGCGTACG |
| | | GTGGCTGCACCATCTGTCTTCATCTTCCCGCCATCTGATGAGCAGTTGAAATCTGGA |
| | | ACTGCCTCTGTTGTGTGCCTGCTGAATAACTTCTATCCCAGAGAGGCCAAAGTACAG |
| | | TGGAAGGTGGATAACGCCCTCCAATCGGGTAACTCCCAGGAGAGTGTCACAGAGCAG |
| | | GACAGCAAGGACAGCACCTACAGCCTCAGCAGCACCCTGACGCTGAGCAAAGCAGAC |
| | | TACGAGAAACACAAAGTCTACGCCTGCGAAGTCACCCATCAGGGCCTGAGCTCGCCC |
| | | GTCACAAAGAGCTTCAACAGGGGAGAGTGT |

### IV. Conjugates

The present disclosure also provides a conjugate comprising the antibody or antibody binding fragment described herein. In some aspects, the conjugate of the disclosure comprises an antibody or antibody fragment thereof described herein and a drug via a linker.

### Drug (Warhead) and Linker

In some aspects, a drug in the antibody drug conjugates described herein is a topoisomerase inhibitor. In some aspects, the drug inhibits topoisomerase I and II.

In some aspects, the drug comprises formula (II):

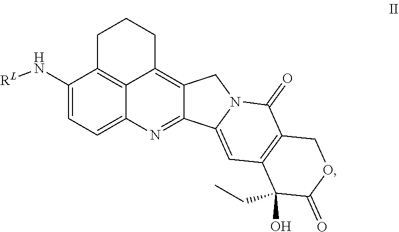

wherein R^{L} is a linker for connection of the drug to a linker unit. In some aspects, a linker unit comprises a linker and an antibody or antigen binding fragment thereof.

The synthesis of a drug of formula II has been described in detail in WO2020/200880A1, which is incorporated herein by reference in its entirety.

In some aspects, a drug molecule of formula II is bound by an antibody or antigen binding fragment thereof as described herein through a linker R^{L}.

In some aspects, a linker R^{L} comprises formula IIa

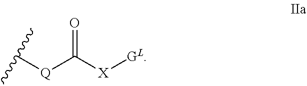

In some aspects, “Q” in the linker R^{L} is

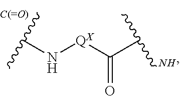

and Q^{X} is such that Q of the linker R^{L} is an amino acid residue, a dipeptide residue, a tripeptide residue or a tetrapeptide residue. In some aspects, the amino acid residue, dipeptide residue, tripeptide residue or tetrapeptide residue are bound to adjacent groups as indicated by the superscripted labels (^{C(═O)} and ^{NH}). For example, “NH” of “Q” is bound to C(═O) of formula IIa and C═O of “Q” is bound to “NH” of formula II.

In some aspects, “X” of the linker of formula IIa is

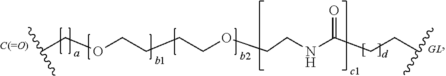

where C(═O) on the left is C═O of formula IIa and GL on the right is GL of formula IIa.

In some aspects, Q is an amino acid residue selected from: Phe, Lys, Val, Ala, Cit, Leu, Ile, Arg, and Trp.

In some aspects, Q is a dipeptide. In some aspects, the dipeptide is any combination of natural amino acids and non-natural amino acids. In some aspects, the dipeptide comprises natural amino acids.

In some aspects, the linker is a cathepsin labile linker. In some aspects, the dipeptide is a recognition site for cathepsin and the dipeptide is the site of action for cathepsin-mediated cleavage.

In some aspects, the dipeptide is selected from: NH-Phe-Lys-C═O, NH-Val-Ala-C═O, NH-Val-Lys-C═O, NH-Ala-Lys-C═O, NH-Val-Cit-C═O, NH-Phe-Cit-C═O, NH-Leu-Cit-C═O, NH-Ile-Cit-C═O, NH-Phe-Arg-C═O, NH-Trp-Cit-C═O, and NH-Gly-Val-C═O; wherein NH represents the N-terminus, and C═O represents the C-terminus of the residue.

In some aspects, Q is a tripeptide. In some aspects, the tripeptide is selected from: NH-Glu-Val-Ala-C═O, NH-Glu-Val-Cit-C═O, NH-αGlu-Val-Ala-C═O, and NH-αGlu-Val-Cit-C═O; wherein NH represents the N-terminus, and C═O represents the C-terminus of the residue. Glu represents the residue of glutamic acid and αGlu represents the residue of glutamic acid when bound via the α-chain.

In some aspects, Q is a tetrapeptide. In some aspects, the tetrapeptide is selected from: NH-Gly-Gly-Phe-Gly C═O; and NH-Gly-Phe-Gly-Gly-C═O, wherein NH represents the N-terminus, and C═O represents the C-terminus of the residue.

In some aspects, the linker R^{L} is formula IIa and X is:

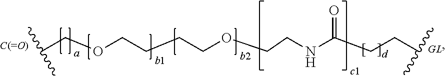

wherein GL is a linker for connecting to the antibody or antigen binding fragment thereof.

In some aspects, the elements a, b1, b2, c1 and d of X comprise a number between 0 and 16. In some aspects, X comprises a between 0 and 5, b1 between 0 and 16, b2 between 0 and 16, c1 between 0 and 1, and d between 0 and 5.

In some aspects, X comprises a between 0 and 3; or 0, or 1.

In some aspects, X comprises b1 between 0 and 8; or 0, 2, 3, 4, 5, or 8.

In some aspects, X comprises b2 between 0 and 8; or 0, 2, 3, 4, 5, or 8.

In some aspects, X comprises c1 of 0, 1, or 2.

In some aspects, X comprises d between 0 and 3; or 1, or 2.

In some aspects, a is 1, c1 is 1, d is 2, and b1 is 2, 3, 4, 5, 6, 7, or 8. In some aspects, a is 2, c1 is 1, d is 1, and b1 is 2, 3, 4, 5, 6, 7, or 8. In some aspects, a is 1, c1 is 2, d is 1, and b1 is 2, 3, 4, 5, 6, 7, or 8. In some aspects, a is 1, c1 is 1, d is 1, and b1 is 2, 3, 4, 5, 6, 7, or 8. In some aspects, a is 0, b1 is 0, c1 is 1, d is 2, and b2 is 0, 2, 3, 4, 5, or 8. In some aspects, a is 1, b2 is 0, c1 is 0, and b1 is 0, 2, 3, 4, 5, or 8. In some aspects, a is 0, b1 is 0, c1 is 0, d is 1, and b2 is 0, 2, 3, 4, 5, or 8. In some aspects, b1 is 0, b2 is 0, c1 is 0, one of a and d is 0, and the other of a and d is 1 or 5. In some aspects, a is 1, b2 is 0, c1 is 0, d is 2, and b1 is 0, 2, 3, 4, 5, or 8. In some aspects, a is 0, b1 is 0, b2 is 8, c1 is 1, and d is 0.

In some aspects, GL is:

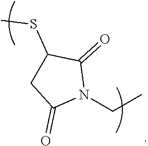

In some aspects, linker R^{L} is:

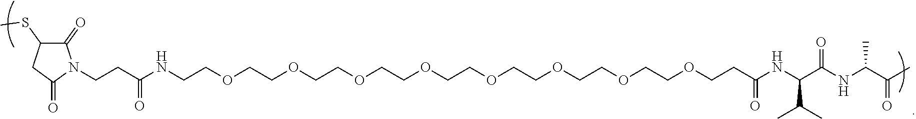

In some aspects, Formula II is

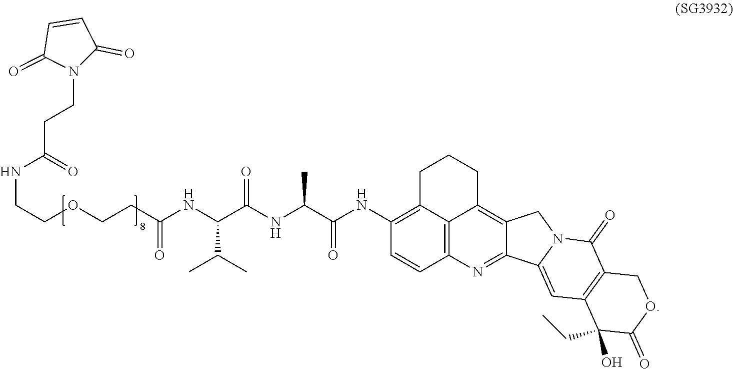

For the avoidance of doubt, the numeral ‘8’ specifies that the structure within boxed parentheses is repeated eight times. Thus, another representation of SG3932 is:

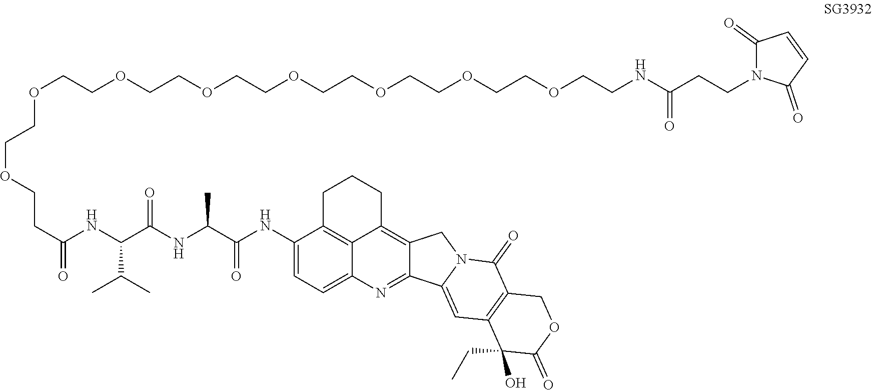 [SMILES: CC[C@@]1(O)C(=O)OCc2c1cc1n(c2=O)Cc2c-1nc1ccc(NC(=O)[C@H](C)NC(=O)[C@@H](NC(=O)CCOCCOCCOCCOCCOCCOCCOCCOCCNC(=O)CCN3C(=O)C=CC3=O)C(C)C)c3c1c2CCC3]

(1-(3-(2,5-dioxo-2,5-dihydro-1H-pyrrol-1-yl)propanamido)-N—((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)-3,6,9,12,15,18,21,24-octaoxaheptacosan-27-amide).

In some aspects, an antibody drug conjugate comprises a drug of formula II, a linker of formula IIa with Q comprising an amino acid residue, dipeptide residue, tripeptide residue or tetrapeptide residue, X comprising an element a between 0 and 5, an element b1 between 0 and 16, element b2 between 0 and 16, an element c1 between 0 and 2, an element d between 0 and 5, a GL of

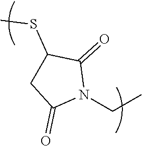

and a CD123 antibody or antigen binding fragment as described herein bound to S of GL.

In some aspects, one drug of formula II and a linker are bound to an antibody or antigen binding fragment described herein forming an antibody drug conjugate. In some aspects, more than one drug of formula II and more than one linker are bound to an antibody or antigen binding fragment described herein forming an antibody drug conjugate.

In some aspects, 1, 2, 3, 4, 5, 6, 7, 8, 9, 10, 11, 12, 13, 14, 15, 16, 17, 18, 19, or 20 drugs of formula II and linkers are bound to an antibody or antigen binding fragment described herein forming an antibody drug conjugate. In some aspects, 1, 2, 3, 4, 5, 6, 7, 8, 9, 10, 11, 12, 13, 14, 15, 16, 17, 18, 19, or 20 drugs of SG3932 are conjugated to an antibody or antigen binding fragment described herein forming an antibody drug conjugate.

In some aspects, about 8 drugs of formula II and linkers are bound to an antibody described herein forming an antibody drug conjugate. In some aspects, about 8 drugs of SG3932 are conjugated to an antibody described herein forming an antibody drug conjugate.

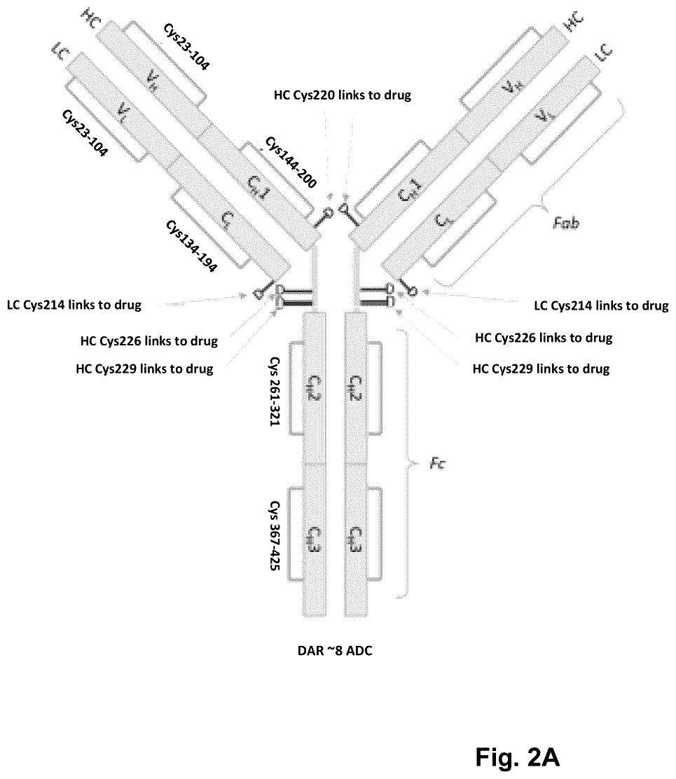

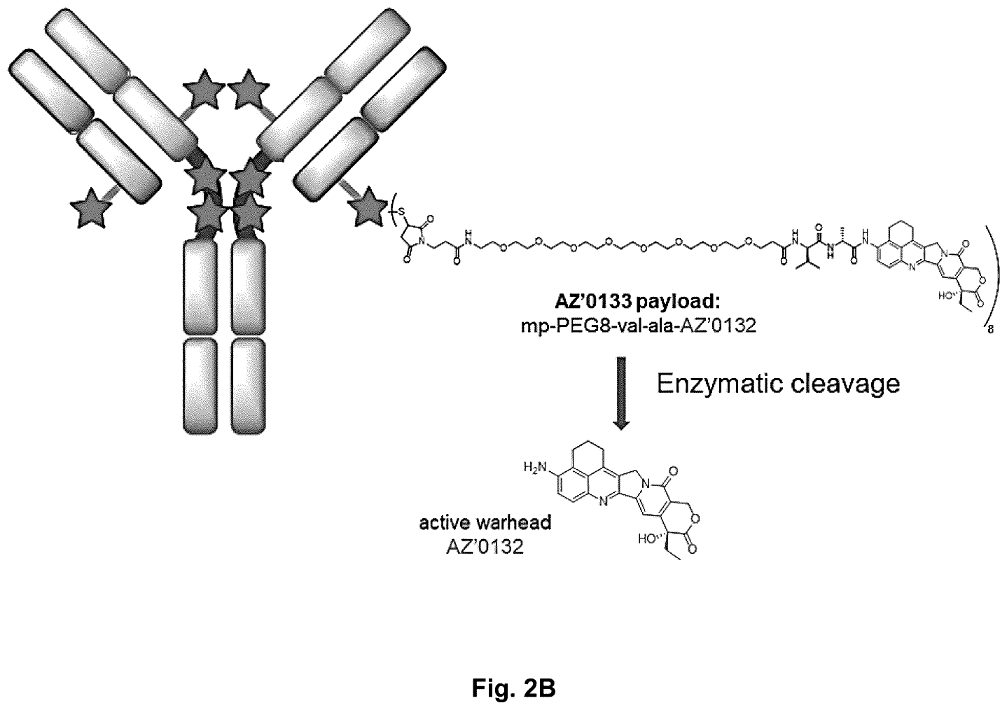

In some aspects, the antibodies described herein are conjugated via reduced cysteine residues to the maleimide of SG3932, resulting in an antibody drug conjugate as represented in FIG. 2B.

In some aspects, the present disclosure provides an anti-CD123 antibody comprising: (a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence of SEQ ID NO: 1, SEQ ID NO: 2, SEQ ID NO: 3, SEQ ID NO: 4, SEQ ID NO: 5, and SEQ ID NO: 6; wherein the antibody is conjugated to one or more compounds represented by the following formula:

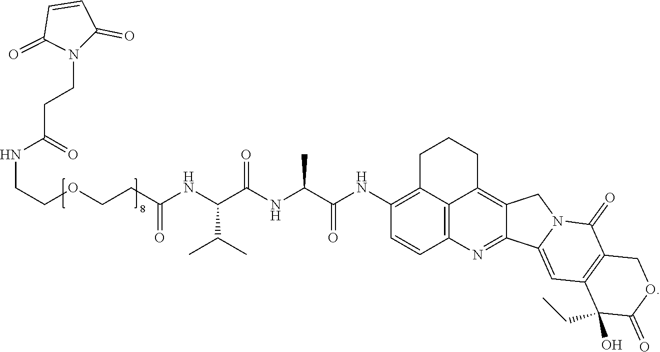

In some aspects, the present disclosure provides an anti-CD123 antibody comprising: (a) a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence as set forth in SEQ ID NO: 1, SEQ ID NO: 2, SEQ ID NO: 3, SEQ ID NO: 4, SEQ ID NO: 5, and SEQ ID NO: 6, respectively; wherein the antibody is conjugated to 1-(3-(2,5-dioxo-2,5-dihydro-1H-pyrrol-1-yl)propanamido)-N—((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)-3,6,9,12,15,18,21,24-octaoxaheptacosan-27-amide.

In some aspects, the present disclosure provides an anti-CD123 antibody comprising: a heavy chain CDR1 (HCDR1), a heavy chain CDR2 (HCDR2), a heavy chain CDR3 (HCDR3), a light chain CDR1 (LCDR1), a light chain CDR2 (LCDR2), and a light chain CDR3 (LCDR3) comprising the amino acid sequence as set forth in SEQ ID NO: 1, SEQ ID NO: 2, SEQ ID NO: 3, SEQ ID NO: 4, SEQ ID NO: 5, and SEQ ID NO: 6, respectively; wherein the antibody is conjugated to about 8 compounds represented by the following formula:

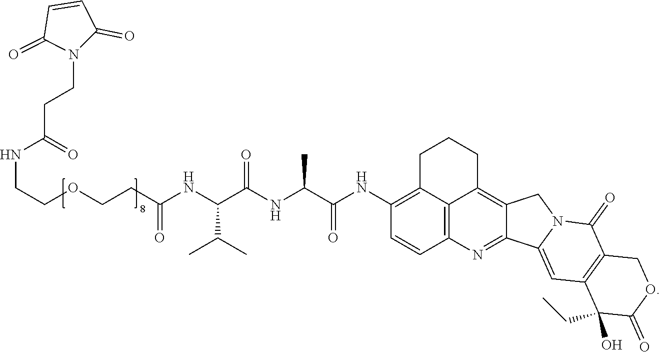

In some aspects, the present disclosure provides an anti-CD123 antibody comprising a heavy chain variable region comprising the amino acid sequence as set forth in SEQ ID NO: 7, and a light chain variable region comprising the amino acid sequence as set forth in SEQ ID NO: 8, wherein the antibody is conjugated to one or more compounds represented by the following formula:

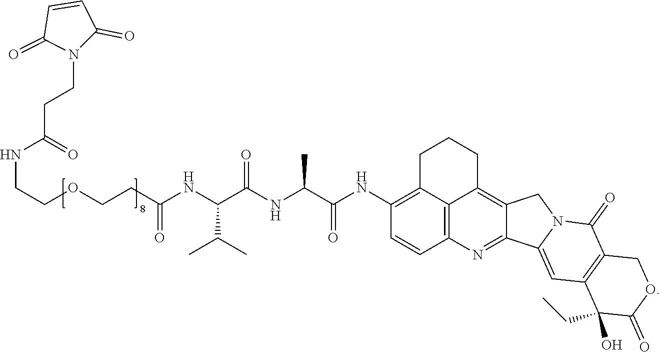

In some aspects, the present disclosure provides an anti-CD123 antibody comprising a heavy chain variable region comprising the amino acid sequence as set forth in SEQ ID NO: 7, and a light chain variable region comprising the amino acid sequence as set forth in SEQ ID NO: 8, wherein the antibody is conjugated to about 8 compounds represented by the following formula:

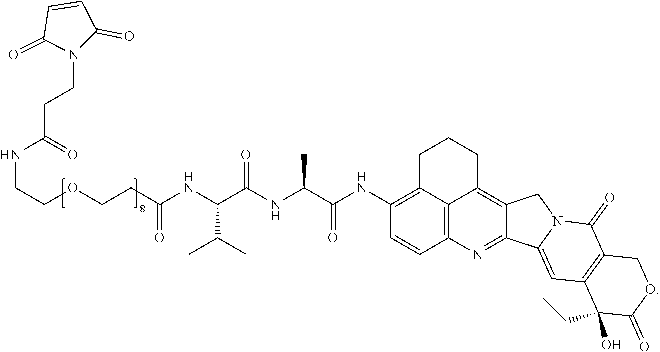

In some aspects, the present disclosure provides an anti-CD123 antibody comprising a heavy chain variable region comprising the amino acid sequence as set forth in SEQ ID NO: 7, and a light chain variable region comprising the amino acid sequence as set forth in SEQ ID NO: 8, wherein the antibody is conjugated to 1-(3-(2,5-dioxo-2,5-dihydro-1H-pyrrol-1-yl)propanamido)-N—((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)-3,6,9,12,15,18,21,24-octaoxaheptacosan-27-amide.

In some aspects, the present disclosure provides an anti-CD123 antibody comprising a heavy chain comprising the amino acid sequence as set forth in SEQ ID NO: 116, and a light chain comprising the amino acid sequence as set forth in SEQ ID NO: 117, wherein the antibody is conjugated to one or more compounds represented by the following formula:

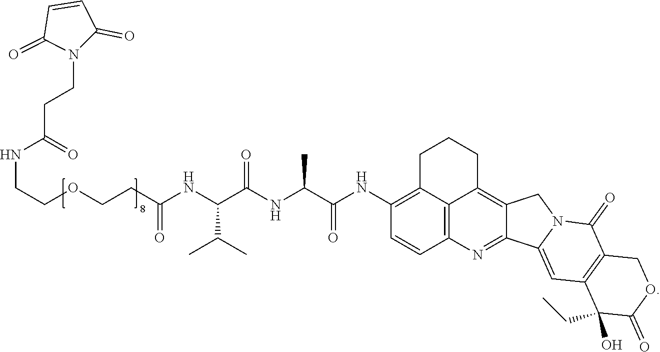

In some aspects, the present disclosure provides an anti-CD123 antibody comprising a heavy chain comprising the amino acid sequence as set forth in SEQ ID NO: 116, and a light chain comprising the amino acid sequence as set forth in SEQ ID NO: 117, wherein the antibody is conjugated to about 8 compounds represented by the following formula:

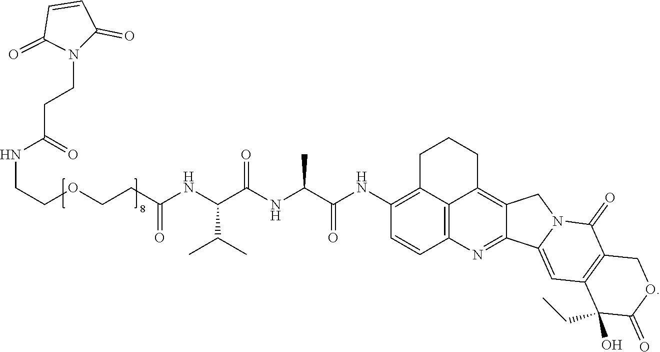

In some aspects, the compounds are conjugated to Cysteine 220 of the heavy chain, Cysteine 226 of the heavy chain, Cysteine 229 of the heavy chain, and Cysteine 214 of the light chain, using EU numbering system.

In some aspects, the present disclosure provides an anti-CD123 antibody comprising CDRs of a heavy chain comprising the amino acid sequence as set forth in SEQ ID NO: 116, and CDRs of a light chain comprising the amino acid sequence as set forth in SEQ ID NO: 117, wherein the CDRs are defined by EU, Kabat, Clothia, and/or IMGT, and wherein the antibody is conjugated to about 8 compounds represented by the following formula:

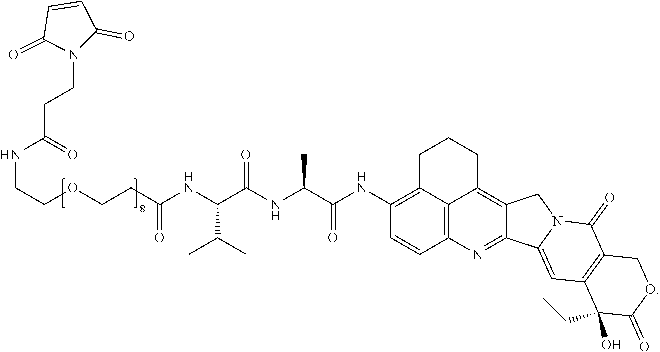

In some aspects, the antibodies disclosed herein (e.g. HT12-GL) are conjugated to a compound of formula

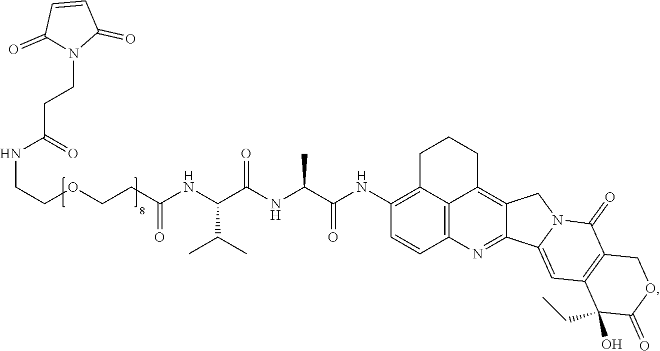

which results in one or more of Cysteine 220 of the heavy chain, Cysteine 226 of the heavy chain, Cysteine 229 of the heavy chain, and Cysteine 214 of the light chain (EU numbering system), forming a succinimidyl thioether of formula

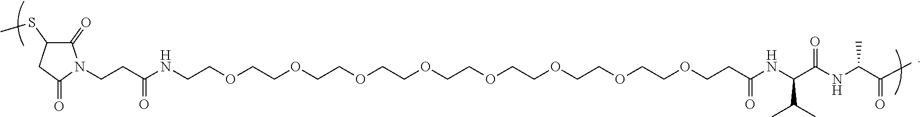

### Synthesis of Topoisomerase I Inhibitors

For completion, certain general synthetic routes for the preparation of preferred topoisomerase I inhibitor(s) will now be described. Further details may be found in the Examples section.

Compounds of formula I where R^{L} is of formula Ia may be synthesised from a compound of Formula 2:

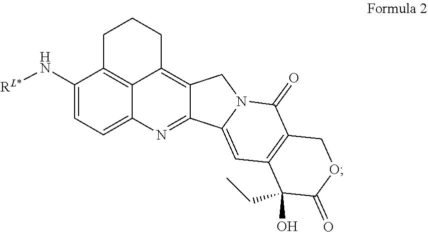

where R^{L}* is -QH by linking a compound of Formula 3:

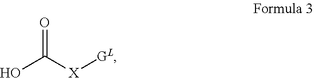

or an activated version thereof.

Such a reaction may be carried out under amide coupling conditions.

Compounds of Formula 2 may be synthesised by the deprotection of a compound of Formula 4:

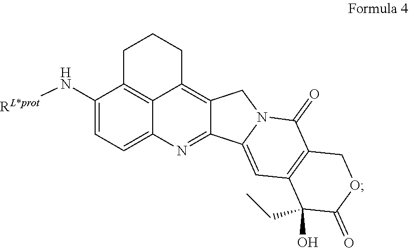

where R^{L*prot} is -Q-Prot^{N}, where Prot^{N} is an amine protecting group.

Compounds of Formula 4 may be synthesised by the coupling of a compound of Formula 5:

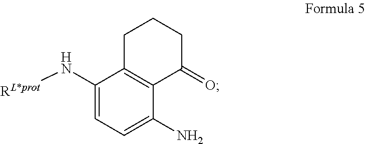

with the compound A3 using the Friedlander reaction.

Compounds of Formula 5 may be synthesised from compounds of Formula 6:

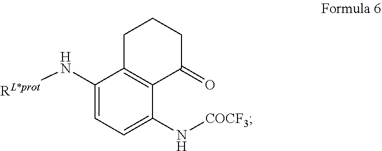

by removal of the trifluoroacetamide protecting group.

Compounds of Formula 6 may be synthesised by coupling: R^{L*prot}—OH to the compound 17.

Compounds of formula I where R^{L} is of formula Ia or Ib may be synthesised from the compound I11 by coupling of the compound R^{L}—OH, or an activated form thereof.

### Amine Protecting Groups:

Amine protecting groups are well-known to those skilled in the art. Particular reference is made to the disclosure of suitable protecting groups in Greene's Protecting Groups in Organic Synthesis, Fourth Edition, John Wiley & Sons, 2007 (ISBN 978-0-471-69754-1), pages 696-871.

### Drug Conjugation

In some aspects, an antibody or antigen fragment thereof as described herein is conjugated to a heterologous agent, e.g., a drug, using site-specific or non-site specific methods of conjugation. In some aspects, more than one drug is conjugated to an antibody or antigen binding fragment thereof. In some aspects, one, two, three, four, five, six, seven, eight or more drug molecules are conjugated to an antibody or antigen binding fragment thereof. In some aspects, all conjugated drug molecules have the same structure. In some aspects, drug conjugation to an antibody or antigen binding fragment described herein is achieved by using predetermined ratios of drug molecules to antibody or antibody fragment molecules during the drug conjugation procedures as described herein.

In some aspects, a drug described herein is conjugated to an antibody or antigen binding fragment described herein using conventional conjugation strategies including, e.g., random conjugation through lysine, cysteine, or a non-natural amino acid.

In some aspects, a drug is randomly conjugated to an antibody or antigen binding fragment thereof by reduction of the antibody or antigen binding fragment, followed by reaction with the drug, with or without a linker attached to the drug.

In some aspects, more than one drug molecule, e.g., a drug molecule of formula II is randomly conjugated to an antibody or antigen binding fragment thereof.

In some aspects, an antibody or antigen binding fragment is reduced using dithiothreitol (DTT), tris(2-carboxyethyl)phosphine (TCEP), or a similar reducing agent. In some aspects, a drug molecule with or without a linker is added at a molar excess to the reduced antibody or antigen binding fragment thereof in the presence of DMSO. In some aspects, the molar excess is adjusted such that more than one drug molecule, e.g., a drug molecule of formula II is conjugated to each reduced antibody or antigen binding fragment thereof.

In some aspects, excess free cysteine is added after conjugation to quench unreacted agent. In some aspects, the reaction mixture is purified and buffer-exchanged into PBS.

In some aspects, a drug molecule is conjugated to an antibody or antigen binding fragment thereof by site-specific conjugation. In some aspects, more than one drug molecule is conjugated to an antibody or antigen binding fragment thereof by site-specific conjugation. In some aspects, site-specific conjugation of a drug molecule to an antibody or antigen binding fragment thereof is performed using reactive amino acid residues at specific positions in the antibody or antigen binding fragment thereof. In some aspects, the site-specific conjugation yields homogeneous preparations of antibody drug conjugates with uniform stoichiometry.

In some aspects, more than one drug molecule is conjugated to an antibody or antigen binding fragment thereof through site-specific conjugation. In some aspects, the more than one drug molecule conjugated to an antibody or antigen binding fragment thereof through site-specific conjugation is a drug molecule of formula II.

In some aspects, a drug molecule with or without a linker is added at a molar excess to an antibody or antigen binding fragment thereof for site-specific conjugation. In some aspects, the molar access is adjusted such that more than one drug molecule is site-specifically conjugated to each antibody or antigen binding fragment thereof.

In some aspects, a drug molecule is conjugated to a side chain of an amino acid. In some aspects, a drug molecule is conjugated to a side chain of an amino acid in an Fc region of an antibody. In some aspects, a drug molecule is conjugated to a side chain of an amino acid in a heavy chain CH3 constant region of an antibody. In some aspects, a drug molecule is conjugated to a side chain of an amino acid in a heavy chain CH2 constant region of an antibody or antigen binding fragment thereof.

In some aspects, a drug molecule is conjugated to a side chain of an amino acid in a hinge region of an antibody or antigen binding fragment thereof. In some aspects, a drug molecule is conjugated to a side chain of an amino acid in a heavy chain CH1 constant region of an antibody or antigen binding fragment thereof.

In some aspects, a drug molecule is conjugated to a side chain of an amino acid in a light chain Cκ region of an antibody or antigen binding fragment thereof. In some aspects, a drug molecule is conjugated to a side chain of an amino acid in a light chain Cλ region of an antibody or antigen binding fragment thereof.

In some aspects, a drug molecule is conjugated to a side chain of an amino acid in at least two of a heavy chain CH1 constant region, a heavy chain CH2 constant region, a heavy chain CH3 constant region, a hinge region, a light chain Cκ region, or a light chain Cλ region of an antibody or antigen binding fragment thereof.

In some aspects, a drug molecule is conjugated to a side chain of an amino acid in at least three of a heavy chain CH1 constant region, a heavy chain CH2 constant region, a heavy chain CH3 constant region, a hinge region, a light chain Cκ region chain, or a light chain Cλ region of an antibody or antigen binding fragment thereof.

In some aspects, a drug molecule is conjugated to a side chain of an amino acid in at least four of a heavy chain CH1 constant region, a heavy chain CH2 constant region, a heavy chain CH3 constant region, a hinge region, light chain Cκ region, or a light chain Cλ region of an antibody or antigen binding fragment thereof.

In some aspects, a drug molecule is conjugated to a side chain of an amino acid in at least five of a heavy chain CH1 constant region, a heavy chain CH2 constant region, a heavy chain CH3 constant region, a hinge region, a light chain Cκ region, or a light chain Cλ region of an antibody or antigen binding fragment thereof.

In some aspects, a drug molecule is conjugated to a side chain of an amino acid in a heavy chain CH1 constant region, a heavy chain CH2 constant region, a heavy chain CH3 constant region, a hinge region, a light chain Cκ region, and a light chain Cλ region of an antibody or antigen binding fragment thereof.

In some aspects, a drug molecule is conjugated to a side chain of an amino acid in more than one heavy chain CH1 constant region. In some aspects, a drug molecule is conjugated to a side chain of an amino acid in more than one heavy chain CH2 constant region. In some aspects, a drug molecule is conjugated to a side chain of an amino acid in more than one heavy chain CH3 constant region. In some aspects, a drug molecule is conjugated to a side chain of an amino acid in more than one hinge region. In some aspects, a drug molecule is conjugated to a side chain of an amino acid in more than one light chain Cκ region. In some aspects, a drug molecule is conjugated to a side chain of an amino acid in more than one light chain Cλ region.

In some aspects, a drug molecule is conjugated to a side chain of an amino acid in at least two heavy chain CH1 constant regions, at least two a heavy chain CH2 constant regions, at least two hinge regions, and at least two light chain constant domain regions (Cκ or Cλ) of an antibody or antigen binding fragment thereof.

In some aspects, a drug molecule is conjugated to a side chain of an amino acid in two heavy chain CH1 constant regions, two a heavy chain CH2 constant regions, two hinge regions, and two light chain constant domain regions (Cκ or Cλ) of an antibody or antigen binding fragment thereof.

In some aspects, a drug molecule is conjugated to Cys220 of the heavy chain of an antibody or antigen binding fragment described herein. In some aspects, a drug molecule is conjugated to Cys226 (EU numbering system) of the heavy chain of an antibody or antigen binding fragment described herein. In some aspects, a drug molecule is conjugated to a Cys229 (EU numbering system) of the heavy chain of an antibody or antigen binding fragment described herein. In some aspects, a drug molecule is conjugated to Cys214 (EU numbering system) of the light chain of an antibody or antigen binding fragment described herein. In some aspects, a drug molecule is conjugated to heavy chain Cys220, heavy chain Cys226, heavy chain Cys229, and light chain Cys214 (EU numbering system).

In some aspects, a drug molecule is conjugated to an antibody or antigen binding fragment thereof through a thiol-maleimide linkage.

In some aspects, where a drug molecule is conjugated to a side chain of a cysteine but only one or few thiol groups are sufficiently reactive for conjugation, additional nucleophilic groups can be introduced into an antibody or antigen binding fragment thereof as described herein through the reaction of lysines with 2-iminothiolane (Traut's reagent) resulting in conversion of an amine to a thiol.

In some aspects, a drug molecule is conjugated to a side chain of a cysteine of an antibody or antigen binding fragment thereof, where the cysteine is part of a disulfide bridge and prior to conjugation the antibody or antigen binding fragment thereof is treated with a reducing agent (such as DTT or TCEP), under partial or total reducing conditions to reduce the thiol groups of the antibody or antigen binding fragment thereof.

In some aspects, reactive thiol groups may be introduced into an antibody or antigen binding fragment thereof by engineering one, two, three, four, or more cysteine residues (e.g., preparing mutant antibodies comprising one or more non-native cysteine amino acid residues). In some aspects, cysteine amino acids may be engineered at reactive sites in an antibody. In some aspects, cysteine amino acids may be engineered at sites that will not form intrachain or intermolecular disulfide linkages.

In some aspects, a drug molecule is conjugated to a side chain of an amino acid of a VH comprising the amino acid sequence set forth in SEQ ID NO: 7. In some aspects, a drug molecule is conjugated to a side chain of an amino acid of a VL comprising the amino acid sequence set forth in SEQ ID NO: 8. In some aspects, a drug molecule is conjugated to a side chain of an amino acid of a heavy chain comprising the amino acid sequence set forth in SEQ ID NO: 116. In some aspects, a drug molecule is conjugated to a side chain of an amino acid of a light chain comprising the amino acid sequence set forth in SEQ ID NO: 117. In some aspects, a drug molecule is conjugated to a side chain of an amino acid of a heavy chain comprising the amino acid sequence set forth in SEQ ID NO: 116 and to a side chain of an amino acid of a light chain comprising the amino acid sequence set forth in SEQ ID NO: 117.

In some aspects, a drug molecule is conjugated to a side chain of an amino acid of a VH comprising the amino acid sequence set forth in SEQ ID NO: 15. In some aspects, a drug molecule is conjugated to a side chain of an amino acid of a VL comprising the amino acid sequence set forth in SEQ ID NO: 16.

In some aspects, the loading (drug/antibody ratio) of an antibody drug conjugate is controlled in several different manners, including: (i) limiting the molar excess of drug linker relative to antibody, (ii) limiting the conjugation reaction time or temperature, and (iii) partial or limiting reductive conditions for cysteine thiol modification.

In some aspects, more than one nucleophilic or electrophilic group of an antibody reacts with a drug molecule described herein and the resulting product may be a mixture of antibody drug conjugates with a distribution of drug molecules attached to the antibody or antigen binding fragment, e.g. 1, 2, 3, 4, 5, 6, 7, or 8 drug molecules conjugated to each antibody or antigen binding fragment thereof.

The drug/drug loading (p) is the average number of drug per antibody or antigen binding fragment. In some aspects, the average number of drugs per antibody or antigen binding fragment is in the range of about 1 to about 20, wherein “about 1” in the context of drug loading (p) means and includes 0 and 2. In some aspects, the range is selected from about 1 to about 10, about 2 to about 10, about 2 to about 8, about 2 to about 6, and about 4 to about 10. In some aspects, the drug loading (p) is about 1, about 2, about 3, about 4, about 5, about 6, about 7, about 8, about 9, about 10, about 11, about 12, about 13, about 14, about 15, about 16, about 17, about 18, about 19, or about 20.

In some aspects, the average number of drugs per antibody in preparations of antibody drug conjugates from conjugation reactions is characterized by conventional means such as UV, reverse phase HPLC, HIC, mass spectroscopy, ELISA assay, and electrophoresis.

In some aspects, a separation, purification, and characterization of homogeneous antibody drug conjugates where p is a certain value is achieved by means including liquid chromatography methods such as polymeric reverse phase (PLRP) and hydrophobic interaction (HIC). In some aspects, preparations of antibody drug conjugates with a single drug loading value (p) are isolated. In some aspects, the single loading value antibody drug conjugates may still be a heterogeneous mixture because the drugs may be attached, via the linker, at different sites on the antibody or antigen binding fragment.

### V. Methods of Using the Antibodies, Antigen Binding Fragments, and Antibody Drug Conjugates

In some aspects, an antibody or antigen binding fragment thereof described herein delivers a payload, e.g., a cytotoxic drug of formula II, to a cell and inhibits or suppresses proliferation of the cell (e.g. of a tumor cell) by at least 10%, at least 20%, at least 30%, at least 40%, at least 50%, at least 60%, at least 70%, at least 80%, at least 90%, at least 95%, at least 96%, at least 97%, at least 98%, at least 99% or about 100% relative to a level of inhibition or suppression in the absence of an antibody drug conjugate. In some aspects, cellular proliferation following antibody drug conjugate delivery is reduced as assayed using art recognized techniques which measure a rate of cell division, a fraction of cells within a cell population undergoing cell division, and/or a rate of cell loss from a cell population due to terminal differentiation or cell death.

In some aspects, an antibody, antigen binding fragment or antibody drug conjugate as described herein (e.g., a CD123 ADC) binds to CD123 on a surface with a K_{D} of 10⁻⁷ M or less, such as approximately less than 10⁻⁸ M, 10⁻⁹ M or 10⁻¹⁰ M. In some aspects, an antibody, antigen binding fragment or antibody drug conjugate as described herein (e.g., a CD123 ADC) binds to human CD123 on a surface with a K_{D} of between about 9×10⁻⁹ M and about 1.7×10⁻¹⁰ M. In some aspects, an antibody, antigen binding fragment or antibody drug conjugate as described herein (e.g., a CD123 ADC) binds to human CD123 on a surface with a K_{D} of about 9.01×10⁻⁹ M or about 1.69×10⁻¹⁰ M.

In some aspects, an antibody, antigen binding fragment or antibody drug conjugate as described herein (e.g., CD123 ADC) binds to cynomolgus CD123 on a surface with a K_{D} of between about 1.2×10⁻⁸ M and about 6.6×10⁻¹⁰ M. In some aspects, an antibody, antigen binding fragment or antibody drug conjugate as described herein (e.g., CD123 ADC) binds to cynomolgus CD123 on a surface with a K_{D} of about 1.17×10⁻⁸ M or about 6.55×10⁻¹⁰ M.

In some aspects, an antibody, antigen binding fragment or antibody drug conjugate as described herein (e.g., CD123 ADC) binds to CD123 on a cell surface, and is internalized into the cell. In some aspects, the antibody, antigen binding fragment or antibody drug conjugate is internalized into a cell with an IC50 at 10 minutes of about 100 ng/ml to about 1 μg/ml, about 100 ng/ml to about 500 ng/ml, about 100 ng/ml to about 250 ng/ml, about 250 ng/ml to about 500 ng/ml, about 350 ng/ml to about 450 ng/ml, about 500 ng/ml to about 1 μg/ml, about 500 ng/ml to about 750 ng/ml, about 750 ng/ml to about 850 ng/ml, or about 900 ng/ml to about 1 μg/ml.

In some aspects, the antibody, antigen binding fragment or antibody drug conjugate is internalized into a cell with an IC50 at 30 minutes of about 100 ng/ml to about 1 μg/ml, about 100 ng/ml to about 500 ng/ml, about 100 ng/ml to about 250 ng/ml, about 250 ng/ml to about 500 ng/ml, about 250 ng/ml to about 350 ng/ml, about 350 ng/ml to about 450 ng/ml, about 500 ng/ml to about 1 g/ml, about 500 ng/ml to about 750 ng/ml, about 750 ng/ml to about 850 ng/ml, or about 900 ng/ml to about 1 μg/ml.

In some aspects, the antibody, antigen binding fragment or antibody drug conjugate is internalized into a cell with an IC50 at 120 minutes of about 50 ng/ml to about 500 ng/ml, about 50 ng/ml to about 100 ng/ml, about 100 ng/ml to about 200 ng/ml, about 200 ng/ml to about 300 ng/ml, about 300 ng/ml to about 400 ng/ml, or about 400 ng/ml to about 500 ng/ml.

In some aspects, the antibody, antigen binding fragment or antibody drug conjugate is internalized into a cell with an IC50 at 8 hours of about 5 ng/ml to about 250 ng/ml, about 10 ng/ml to about 25 ng/ml, about 25 ng/ml to about 50 ng/ml, about 50 ng/ml to about 100 ng/ml, about 100 ng/ml to about 150 ng/ml, about 150 ng/ml to about 200 ng/ml, or about 200 ng/ml to about 250 ng/ml.

In some aspects, the antibody, antigen binding fragment, or antibody drug conjugate is internalized into an endosome of a cell within about 1 hour of exposure of the cell to the antibody, antigen binding fragment or antibody drug conjugate. In some aspects, provided is a method of delivering a payload, e.g., a drug of formula II to an endosome of a cell, the method comprising contacting a cell with an antibody drug conjugate to deliver the payload to an endosome of the cell within about 1 hour of contacting the cell with the antibody drug conjugate.

In some aspects, an antibody, antigen binding fragment or antibody drug conjugate is internalized into a lysosome of a cell within between about 0.5 hour and about 5 hours of exposure of a cell to the antibody, antigen binding fragment or antibody drug conjugate. In some aspects, provided is a method of delivering a payload, e.g., a drug of formula II to a lysosome of a cell, the method comprising contacting a cell with an antibody drug conjugate to deliver the payload to a lysosome of the cell within about 0.5 hours to about 5 hours of contacting the cell with the antibody drug conjugate.

In some aspects, provided is a method of increasing caspase activity in a cell, the method comprising contacting a cell that expresses CD123, e.g., a CD123 expressing leukemic cell, with an antibody drug conjugate described herein.

In some aspects, a method of delivering a cytotoxic drug to a cell is provided. In some aspects, the method comprises contacting a cell that expresses CD123, e.g., a CD123 expressing leukemic cell, with an antibody drug conjugate described herein.

In some aspects, a method of delivering a cytotoxic drug to a cell that does not express CD123 is provided. In some aspects, the method comprises contacting a cell that expresses CD123 with an antibody drug conjugate described herein, wherein the drug is released in the CD123 expressing cell and exerts a cytotoxic effect by bystander killing on the cell that does not express CD123. In some aspects, the method comprises delivering a cytotoxic drug to a tissue in a subject, wherein the tissue comprises CD123 expressing cells and cells that do not express CD123, wherein the drug is released in the CD123 expressing cell and exerts a cytotoxic effect by bystander killing on the cells of the tissue that do not express CD123. In some aspects, an optimal bystander cell killing is achieved in a tissue when the ratio of CD123 expressing cells to CD123 non-expressing cells is between about 50:50 and about 90:10.

In some aspects, provided is a method of treating a pre-malignant or malignant disease, the method comprising administering to a subject in need of treatment a therapeutically-effective amount of an antibody drug conjugate described herein.

In some aspects, provided is a method of treating leukemia, the method comprising administering to a subject in need of treatment a therapeutically-effective amount of an antibody drug conjugate described herein that efficiently inhibits bone marrow colony forming unit (CFU) activity of leukemic bone marrow cells. In some aspects, a therapeutically-effective amount of an the antibody drug conjugate described herein when administered to a subject having leukemia is an amount that reduces the number of leukemic cells in whole blood and bone marrow of the subject.

Advantageously, the antibody drug conjugate described herein does not affect viability of healthy hematopoietic stem cell, healthy granulocyte-monocyte progenitor cells, healthy differentiated hematopoietic cells, or CFU activity of normal bone marrow cells.

In some aspects, the antibody drug conjugate described herein is used in a cellular or tissue environment where endothelial cell damage is to be avoided and/or tight junction integrity is to be preserved.

In some aspects, the antibody drug conjugate described herein is administered to a subject having a CD123 expressing tumor, wherein the CD123 antibody drug conjugate reduces tumor volume.

In some aspects, the antibody drug conjugate described herein is administered to a subject having a proliferative condition including a premalignant or malignant proliferative condition where the pre-malignant or malignant cells express CD123. The pre-malignant or malignant condition includes, but is not limited to, acute myeloid leukemia (AML), acute lymphoblastic leukemia (ALL), blastic plasmacytoid dendritic cell leukemia, hairy cell leukemia, systemic mastocytosis, Hodgkin lymphoma, large B cell lymphoma, chronic myelomonocytic leukemia, chronic lymphocytic leukemia (CLL), and myelodysplastic syndrome.

In some aspects, the antibody drug conjugate described herein is administered to a subject alone or in combination with additional treatments, either simultaneously or sequentially. Examples of other treatments and therapies include, but are not limited to, chemotherapy (the administration of active agents, including, e.g. drugs); surgery; and radiation therapy.

In some aspects, the additional treatment is an additional cancer therapy including, for example, an alkylating agent, antimetabolite, spindle poison plant alkaloid, cytotoxic/antitumor antibiotic, photosensitizer, and/or kinase inhibitor.

In some aspects, the method of treatment further comprises administering, e.g., a protein kinase inhibitor such as a MEK inhibitor; a lipid kinase inhibitor; an antisense oligonucleotide, particularly an oligonucleotide that inhibits the expression of genes in signaling pathways implicated in aberrant cell proliferation, for example, PKC-alpha, Raf and H-Ras; a vaccine; or an anti-angiogenic agent.

### III. Compositions, Formulations, and Kits

In some aspects, the antibody drug conjugate described herein is present in a pharmaceutical composition. In some aspects, a pharmaceutical composition comprises, in addition to the antibody drug conjugate, a pharmaceutically acceptable excipient, carrier, buffer, stabilizer or other materials known to those skilled in the art. Such materials should be non-toxic and should not interfere with the efficacy of the antibody or antigen binding fragment thereof or the activity of the drug of the antibody drug conjugate. The precise nature of the carrier or other material will depend on the route of administration.

In some aspects, a drug of the disclosure is a salt. It may be convenient or desirable to prepare, purify, and/or handle a corresponding salt of the active drug, for example, a pharmaceutically-acceptable salt. Examples of pharmaceutically acceptable salts are discussed in Berge, et al., J. Pharm. Sci., 66, 1-19 (1977).

In some aspects, a composition comprises an antibody conjugate as described herein and a solvate. It may be convenient or desirable to prepare, purify, and/or handle a corresponding solvate of the active drug. In some aspects, the solvate refers to a complex of solute (e.g. active drug, salt of active drug) and solvent.

Also provided are kits according to the present disclosure that comprise an antibody drug conjugate or an antigen binding fragment drug conjugate as described herein and optionally instruction, e.g., administration instructions. In some aspects, a kit comprises multiple packages of single-dose pharmaceutical compositions each containing an effective amount of an antibody drug conjugate or an antigen binding fragment drug conjugate for a single administration in accordance with the administration instructions. In some aspects, the kit also includes one or more additional cancer therapeutics. In some aspects, the kit includes instruments or devices necessary for administering the pharmaceutical composition(s).

### EXAMPLES

### Example 1-CD123 Expression in Healthy Donor Bone Marrow Mononuclear Cells (BMMCs) and AML Patient BMMCs

Primary healthy donor samples (AllCells) and AML patient samples (Discovery Life Sciences and ABS) were subjected to immunophenotypic analysis using multiparametric flow cytometry. Cell subsets were identified using parameters defined in Table 2. The antibody binding capacity of CD123 was generated using Bangs Laboratories Quantum Simply Cellular Beads (Cat #816).

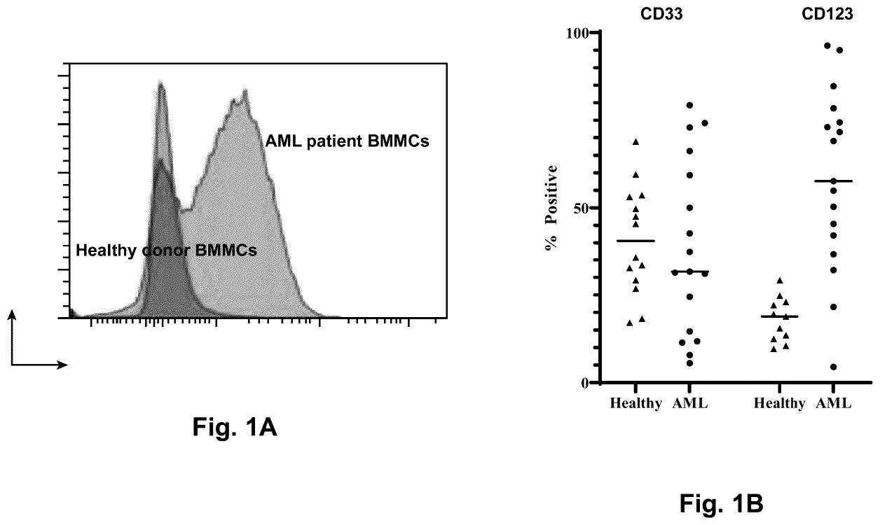

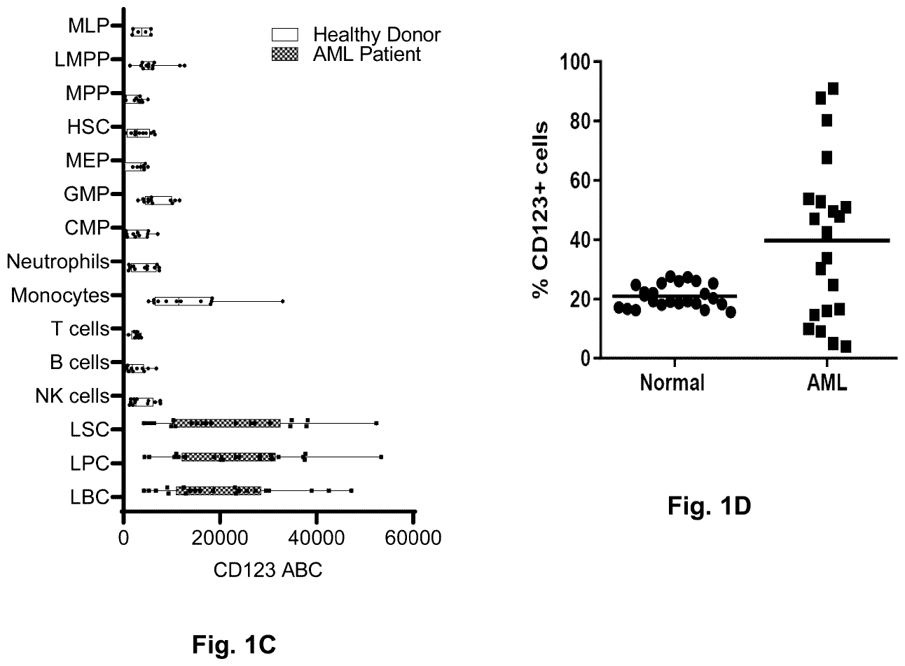

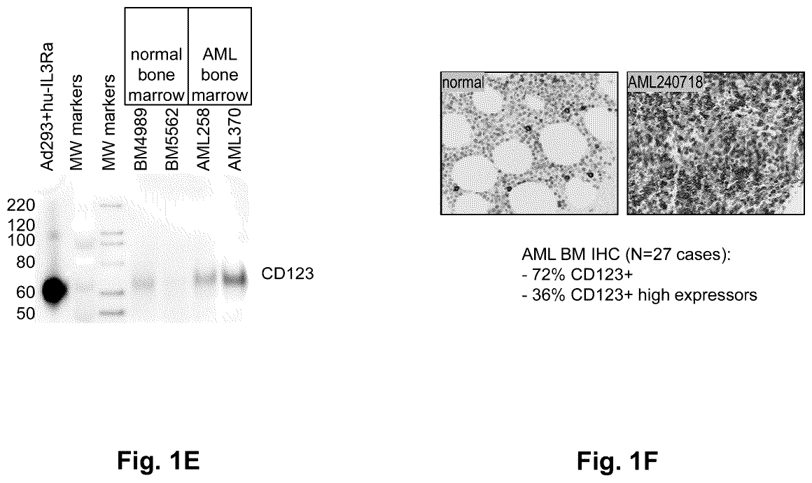

CD123 was preferentially expressed on AML patient BMMCs compared to healthy donor BMMCs (FIG. 1A). A larger difference between CD123 expression levels on healthy donor BMMCs and AML patient BMMCs was observed for CD123 compared to CD33 making CD123 a good candidate for AML cell targeting (FIG. 1B).

HT12-GL antibody binding capacity (ABC) was assessed on healthy donor BMMCs and AML patient leukemic populations. Cell subsets were further defined as shown in Table 2. Expression of CD123 was low on healthy bone marrow cells and elevated in AML patient bone marrow cells (FIG. 1C).

TABLE 2

Healthy Donor Samples
HSC: Lineage⁻ CD34⁺ CD38⁻ CD90⁺ CD45RA⁻
MPP: Lineage⁻ CD34⁺ CD38⁻ CD90⁻ CD45RA⁻
LMPP: Lineage⁻ CD34⁺ CD38⁻ CD90⁻ CD45RA⁺ CD10⁻
MLP: Lineage⁻ CD34⁺ CD38⁻ CD90⁻ CD45RA⁺ CD10⁺
CMP: Lineage⁻ CD34⁺ CD38⁺ CD135⁺ CD45RA⁻
GMP: Lineage⁻ CD34⁺ CD38⁺ CD135⁺ CD45RA⁺
MEP: Lineage⁻ CD34⁺ CD38⁺ CD135⁻ CD45RA⁻
B Cell: CD19⁺
T Cell: CD3⁺
NK Cell: CD56⁺
Neutrophil: CD66b⁺ Mac−1⁺
Monocyte: CD14⁺ Mac−1⁺
Leukemic Patient Disease Subsets
LBC: SSC^{Med-Low} CD45^{Int}
LSC: SSC^{Med-Low} CD45^{Int}, CD34⁺, CD38⁻
LPC: SSC^{Med-Low} CD45^{Int}, CD34⁺, CD38⁺
*Lineage = CD14, CD3, CD19, CD20, CD66b & CD56

Primary AML and normal bone marrow samples were obtained from proteogenix. Cells were thawed at 37° C. and washed 2× with ice cold FACS buffer (Phosphate Buffer Solution+2% Fetal Bovine Serum). CD123 antibody conjugated to BV605 (biolegend, clone 6H6) was used to detect CD123+ cells. Primary bone marrow cells were incubated in 100 μL of staining solution (FACS buffer+5 μL of antibody) for 30 minutes on ice. After incubation, cells were washed 3× with FACS buffer and resuspended in 200 μL of FACS buffer for flow cytometry analysis on a LSRII (BD bioscience) instrument. Data collected from flow cytometry was analyzed using the flowjo software (BD bioscience).

A higher percentage of CD123+ cells was observed in AML patient bone marrow samples compared to healthy bone marrow (FIG. 1D).

CD123 (IL3Rα) was assessed in cell lysates by western blotting. Whole cell lysates from Ad293 cells expressing human IL3Rα were prepared by washing cells once with ice-cold phosphate-buffered saline (PBS) and then lysing the cells by adding Laemmli Reducing buffer (BP-111R; Boston BioProducts). Cell pellets from normal and AML bone marrow patient samples were directly lysed by adding Laemmli Reducing buffer followed by vortexing. After a brief incubation, cell lysates were collected, 10 to 20 ml were loaded onto Bis NuPAGE Novex Bis-Tris gels (Invitrogen) and proteins transferred to PVDF membranes (Invitrogen). Membranes were blocked with 5% nonfat dry milk and 0.1% Tween 20 (Sigma) in TBS (pH 7.4) (TBST) and incubated overnight at 4° C. with antibodies to IL3Rα (NCL-L-CD123, Leica) and actin (A1978, Sigma). Membranes were washed in TBST and then incubated for 1 hour with horseradish peroxidase (HRP)-conjugated streptavidin secondary antibodies (GE Healthcare). After washing, SuperSignal West Femto Chemiluminescent Substrate and SuperSignal West Pico Chemiluminescent Substrate (Pierce/Thermo Scientific) were used to capture and analyze protein bands in an ImageQuant LAS4000 instrument (GE Healthcare).

Increased CD123 (IL3Rα) expression was observed by western blotting in AML patient bone marrow compared to healthy bone marrow (FIG. 1E).

CD123 (IL3a) was assessed by immunohistochemistry (IHC). Anti-IL3Rα (CD123) mouse monoclonal antibody (clone [BR4MS]) used for IHC was obtained from Leica Biosystems (catalog #NCL-L-CD123) and diluted in Antibody Diluent with background reducing agents (Agilent, catalog #S3022). IHC was performed on a Leica Bond Autostainer instrument (Leica Biosystems, Buffalo Grove, IL, USA). Sections of formalin-fixed paraffin-embedded (FFPE) tissue were cut at 4 μm, placed on StarFrost® microscope slides, and loaded onto the auto-stainer. Slides were antigen-retrieved with Bond Epitope Retrieval Solution 2 (Leica Biosystems, catalog #AR9640). Then, HRP was blocked with Peroxide Block (Leica Biosystems, catalog #DS9800) and then incubated with primary antibody. Next, samples were incubated with rabbit anti-mouse IgG secondary Post Primary (Leica Biosystems, catalog #DS9800), then incubated with anti-rabbit IgG HRP post-secondary Polymer (Leica Biosystems, catalog #DS9800). Staining was visualized by brown 3,3′ diaminobenzidine (DAB) with the BOND Polymer Refine Detection Kit (Leica Biosystems, catalog #DS9800). Sections were counterstained with Hematoxylin (Leica Biosystems, catalog #DS9800). Finally, sections were dehydrated in graded ethanol, cleared in xylene, and cover-slipped.

Immunohistochemistry images showed high prevalence of CD123 positivity in AML patient bone marrow compared to healthy bone marrow (FIG. 1F).

### Example 2—Generation of CD123 Antibodies

The anti-CD123 antibodies disclosed herein were discovered via hybridoma and Immune Replica technology using the humanized transgenic Abelixis and Del-1 mouse strains (engineered to express fully human antibody variable domains). Mice were immunized with HEK293 cells that over expressed human CD123 extracellular domain (ECD) using a repetitive immunization multiple site (RIMMS) strategy and were boosted with the recombinant human and cynomolgus monkey CD123 extracellular domain (ECD). B-cells were harvested from the draining lymph nodes and spleen. One million B-cells from each mouse strain was transferred to the Immune-replica campaign, a method for antibody selection adapted from that described by Rajan, S., Kierny, M. R., Mercer, A. et al. Recombinant human B cell repertoires enable screening for rare, specific, and natively paired antibodies. Commun Biol 1, 5 (2018). The remaining B-cells were mixed with SP2/0 myeloma cells and fused using an electrofusion apparatus to generate 6252 viable hybridoma clones. To identify human and cyno cross-reactive CD123 specific binders, supernatant from the hybridomas clones were screened for binding to huCD123 ECD antigen, CynoCD123 ECD antigen, human and Cyno HEK293 CD123 overexpressing cells. Clones that bound to parental HEK293 or Jurkat cells were deemed to be non-specific binders. Of the hybridomas screened, 992 clones bound specifically to the huCD123 ECD, CynoCD123 ECD, HEK-huCD123, and cynoCD123-HEK cells but did not bind to parental HEK-293 cells or Flt3 antigen, used as negative controls. Antibodies from the 992 hybridomas were screened for their ability to induce internalization of hCD123 on HEK293 overexpressing cells and ˜90 showed internalization within 12 hours. Binning of the clones were done by a competition ELISA against two previously generated anti-CD123 antibodies; a minimum of four bins were identified. To confirm binding to CD123 parental cells, the supernatants were also screened for binding to Molm13 (CD123 high expressing) and E02-1 Pb (CD123 low expressing) cell lines. Ninety-one clones of the 992 bound to CD123 parental cells MOLM-13 and EOL-1, with no binding to Jurkat cells (CD123 negative). To identify the heavy and light chain variable region sequence, cDNA from there hybridomas were generated and nGen sequencing was used to retrieve the gene sequences. Twenty four of the 91 clones were initially analyzed, and any duplicate antibodies sequences were consolidated or antibodies with sequence liabilities that could not be fixed were eliminated. Sixteen useful unique antibody genes were successfully retrieved. The 16 parental antibodies sequences were analyzed and germlined, if needed, and any sequence liabilities removed. This process resulted in 31 clones with optimized or parental sequences. In some cases, germlining resulted in a change in the affinity. Twenty-six mAb were made and expressed and purified for the screen. These 26 mAbs were characterized in different cellular binding, internalization, and cytotoxicity assays as described herein.

### Example 3—CD123 Antibody Drug Conjugation

A 50 mM solution of Tris(2-carboxyethyl)phosphine (TCEP) in phosphate-buffered saline pH 7.4 (PBS) was added (6 molar equivalent/antibody, 320 micromoles, 6400 μL) to a 1000 mL solution of antibody (e.g., HT12-GL, J13, various HT clones, antibodies described herein) (8 g, 53.3 micromoles) in reduction buffer containing PBS and 1 mM ethylenediaminetetraacetic acid (EDTA) and a final antibody concentration of 8 mg/mL. The reduction mixture was allowed to react at 37° C. temperature for 2 hours (or until full reduction is observed by UHPLC) in an orbital shaker with gentle (60 rpm) shaking.

A drug of SG3932 was added as a DMSO solution (10 molar equivalent/antibody, 533 micromoles, in 100 mL DMSO) to 1000 mL of this reduced antibody solution (8 g, 53.3 micromoles) for a 10% (v/v) final DMSO concentration. The solution was mixed for 2 hours at room temperature, then the conjugation was quenched by addition of N-acetyl cysteine (134.4 micromoles, 1.34 mL at 100 mM).

Macromolecular aggregates, conjugation reagents, including cysteine quenched drug were removed using ceramic hydroxyapatite Type II chromatography (CHT) as described previously (Thompson et al., J. Control Release, 236: 100-116 (2016)). Site-specific ADCs were formulated in 25 mM Histidine-HCl, 7% sucrose, 0.02% polysorbate-80, pH 6.

To determine monomeric content, aggregates, and fragments, analytical size-exclusion chromatography (SEC-HPLC) was performed using 100 μg (100 μL volume) of antibodies or ADCs, which were loaded into a TSKgel G3000WXL column (Tosoh Bioscience, Tokyo, Japan). The mobile phase was composed of 0.1 M sodium sulfate, 0.1 M sodium phosphate, and 10% isopropanol, pH 6.8. The flow rate was 1 mL/min, and each analysis was carried out for 20 minutes at room temperature. Hydrophobic interaction chromatography (HIC-HPLC) was used to assess conjugation and drug load distribution, and was performed using a butyl-non porous resin (NPR) column (4.6 m IDx3.5 cm, 2.5 m, Tosoh Bioscience). The mobile phase A was composed of 25 mM Tris-HCl, 1.5 M (NH₄)₂SO₄, pH 8.0; and the mobile phase B was composed of 25 mM Tris-HCl and 5% isopropanol, pH 8.0. 100 L of antibodies or ADCs at a concentration of 1 mg/mL were loaded and eluted at a flow rate of 1 mL/min with a gradient of 5% B to 100% B over 13 min. Reduced reverse phase chromatography (rRP-HPLC) was used to confirm chain-specific conjugation. The antibodies and ADCs were reduced at 37° C. for 20 minutes using 42 mM dithiothreitol (DTT) in PBS (pH 7.2). 10 μg of reduced antibodies or ADCs were loaded onto a polymeric reverse phase media (PLRP-S) 1000 A column (2.1×50 mm) (Agilent Technologies, Santa Clara, Calif.) and eluted at 80° C. at a flow rate of 1 mL/min with a gradient of 5% B to 100% B over 25 minutes (mobile phase A: 0.1% Trifluoroacetic acid in water; mobile phase B: 0.1% Trifluoroacetic acid in acetonitrile).

Conjugation at the heavy and light chains and drug:antibody ratios (DAR) were determined by reduced liquid chromatography mass spectrometry analysis (rLCMS) performed on an Agilent 1290 series uHPLC coupled to an Agilent 6230 TOF (Agilent Technologies, Santa Clara, Calif). 2 μg of reduced antibodies or ADCs were loaded onto a ZORBAX rapid resolution high definition (RRHD) 300-Diphenyl column (2.1×50 mm, 1.8 μm) (Agilent Technologies, Santa Clara, Calif.) and eluted at a flow rate of 0.5 mL/min using a step gradient of 80% B after 2.1 min (mobile phase A: 0.1% Formic acid in water and mobile phase B: 0.1% Formic acid in acetonitrile). A positive time-of-flight MS scan was acquired, and data collection and processing were carried out using MassHunter software (Agilent Technologies, Santa Clara, Calif.). DAR was calculated using the rLCMS data as described in Thompson et al., supra. HT12-GL ADC was obtained with DAR 8. FIGS. 2A and 2B.

### Example 4—CD123 Antibody Affinity

The affinity of CD123 antibodies described herein was determined using Biolayer interferometry.

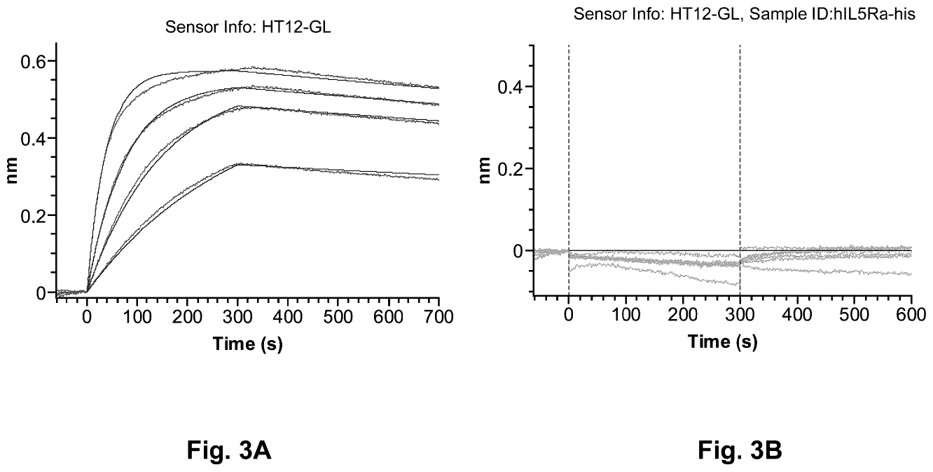

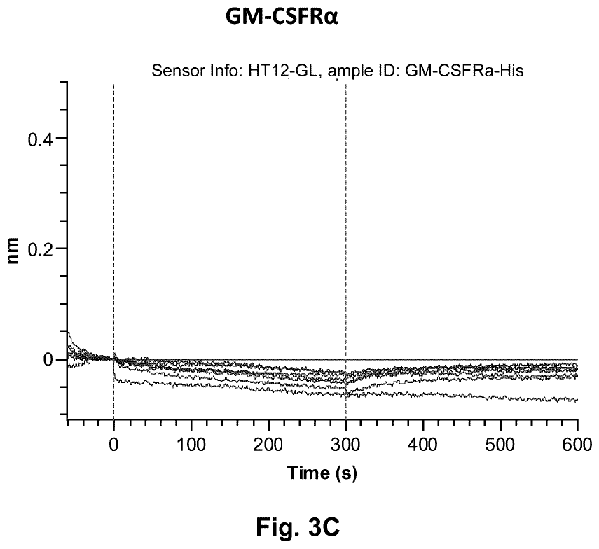

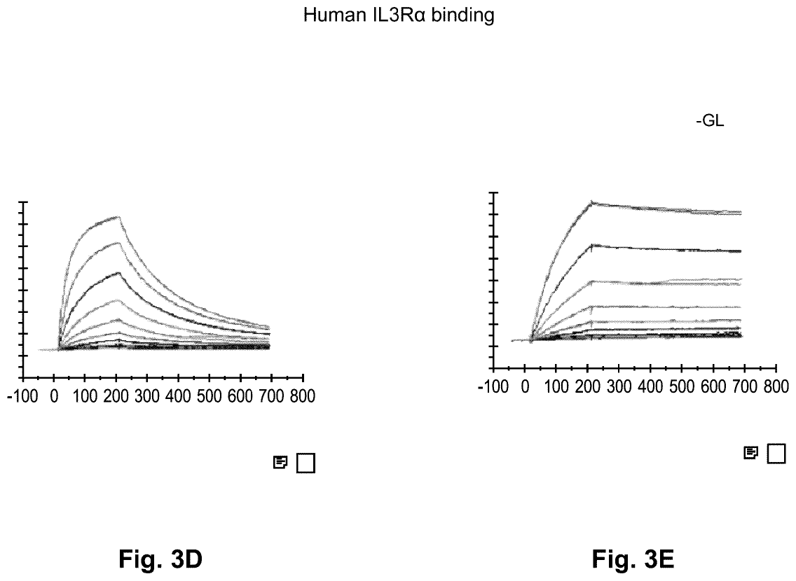

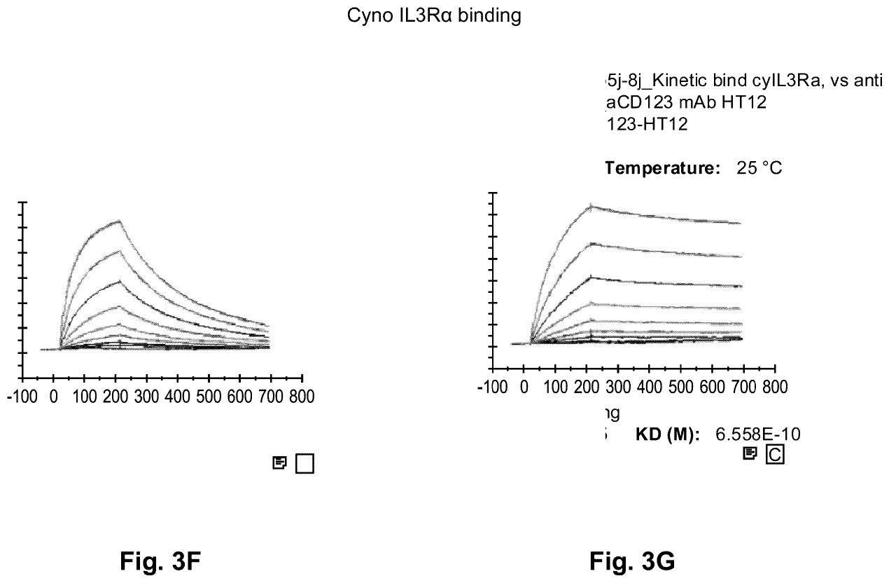

Biolayer interferometry binding experiments were performed using an Octet RED384 instrument, with all measurements made at 30° C. AHC biosensor probes were soaked in kinetics buffer (PBS pH 7.2+0.02% Tween-20, 0.1% BSA, 0.05% sodium azide) for 10 minutes, followed by baseline signal measurement in kinetics buffer for 60 seconds. Antibody HT12-GL was loaded from a 2 μg/mL solution onto AHC biosensors for 120 sec. For binding to hIL3-Rα, the antibody-loaded biosensor tips were dipped in binding buffer for 60 sec (baseline), followed by immersion in a solution containing dilutions of hIL3-Rα (10-0.16 μg/mL) for 300 sec (association), and subsequent immersion in kinetics buffer for 400 sec (dissociation). For binding to hIL5-Rα and GM-CSFRa, all steps were the same except the concentration of antigen was increased (50-1.56 μg/mL) and the dissociation step was decreased to 300 sec. The signal from a buffer only reference sample was subtracted, and all curves were fit with a 1:1 binding model to determine K_{D} values. HT12-GL showed an affinity for hIL3Rα of 0.31 nM, and no measurable binding against related proteins hIL5-Rα and GM-CSFRα (FIGS. 3A-3C and 3E). HT12-GL also bound cynomolgus IL3Rα (FIG. 3G). Antibody J13 also showed affinity for human IL3Rα and cynomolgus IL3Rα (FIG. 3D and FIG. 3F).

### Example 5—CD123 Antibody HT12-GL Cell Binding Capacity

HT12-GL binding to AD293 cells expressing CD123 (IL3Rα) from different species were assessed. AD293 cells were maintained in DMEM (Dulbecco's Modified Eagle Medium) containing 10% heat-inactivated fetal bovine serum (FBS) and were cultured in a humidified atmosphere of 5% CO2 and at a temperature of 37° C. On the day of plating, cells were spun down at 1500 rpm for 3 minutes, counted, and resuspended to a final concentration of 0.3×10⁶ cells/ml. Two hundred μl of cells were plated to wells of 96 well microtiter plate. Plates were spun down at 1500 rpm for 3 minutes and flicked to remove media. One hundred μl of FACs buffer (PBS [Phosphate Buffer Saline]+2% FBS) was added to the wells. Plates were then spun again as above followed by addition of primary antibody in 100 μl of FACS buffer. The primary antibody was HT12-GL labeled with Alexa 647. Antibody concentration started at 12 μg/ml followed by six 1:3 dilutions (no antibody control was also run). Plates were incubated at 4° C. for 30 minutes. Following incubation plates were washed 3 times by spinning down at 1500 rpm for 3 minutes, flicked and 100 μl of FACS buffer were added. Following a final wash, 100 μl of FACS buffer containing live/dead DAPI (diamidino-2-phenylindole) stain was added to the plates. The cells were analyzed on a BD Bioscience LSRFortessa by collecting 10000 cells. Flow data was analyzed in FlowJo software.

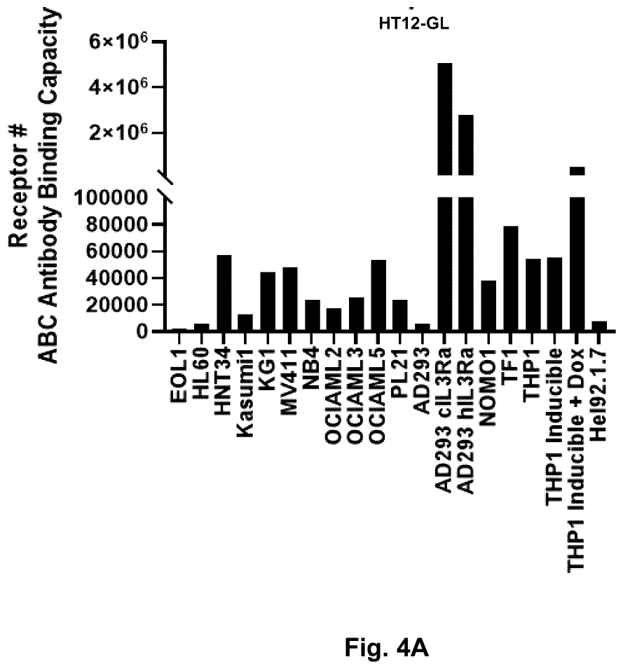

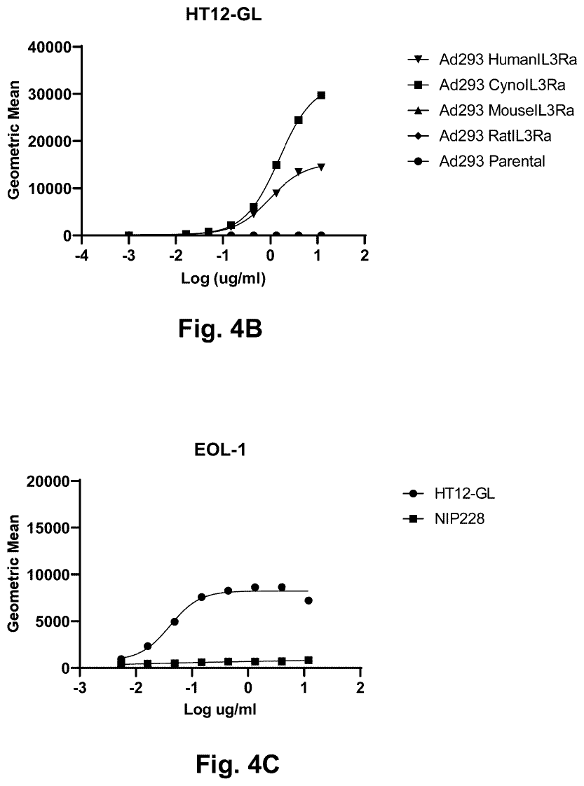

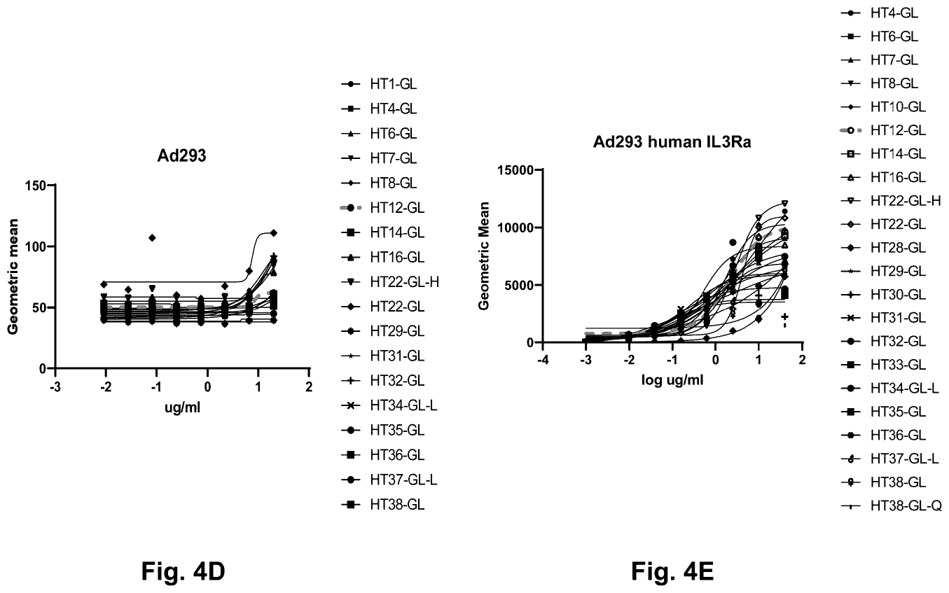

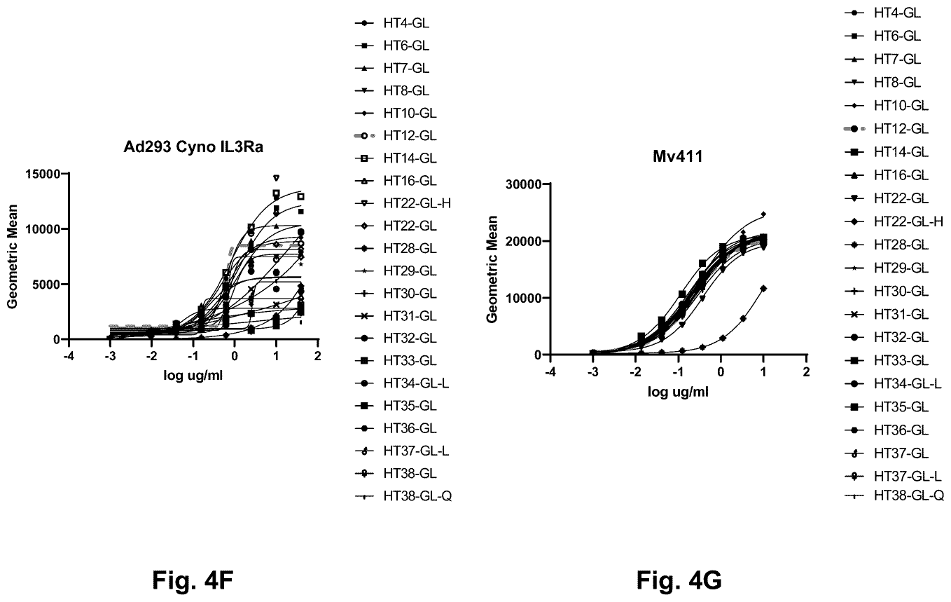

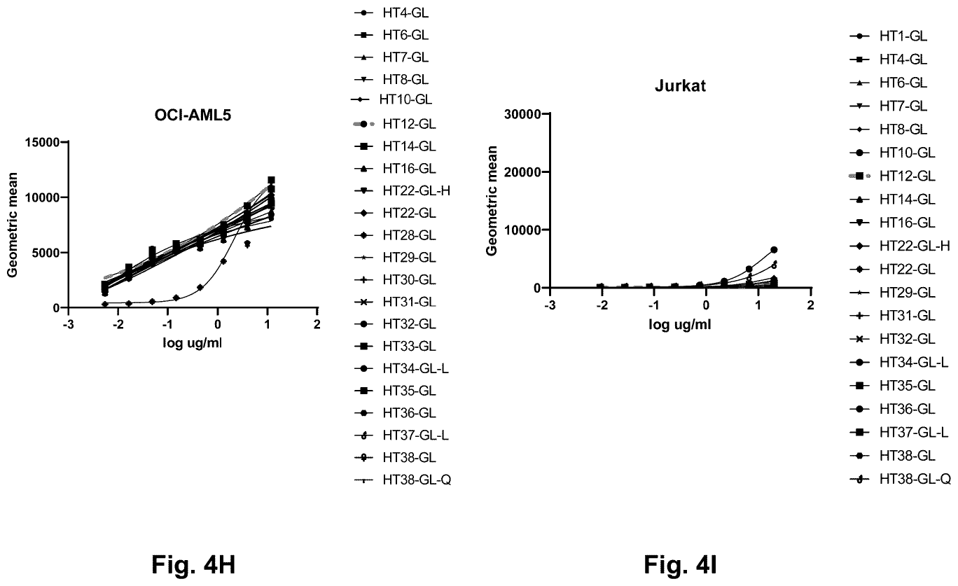

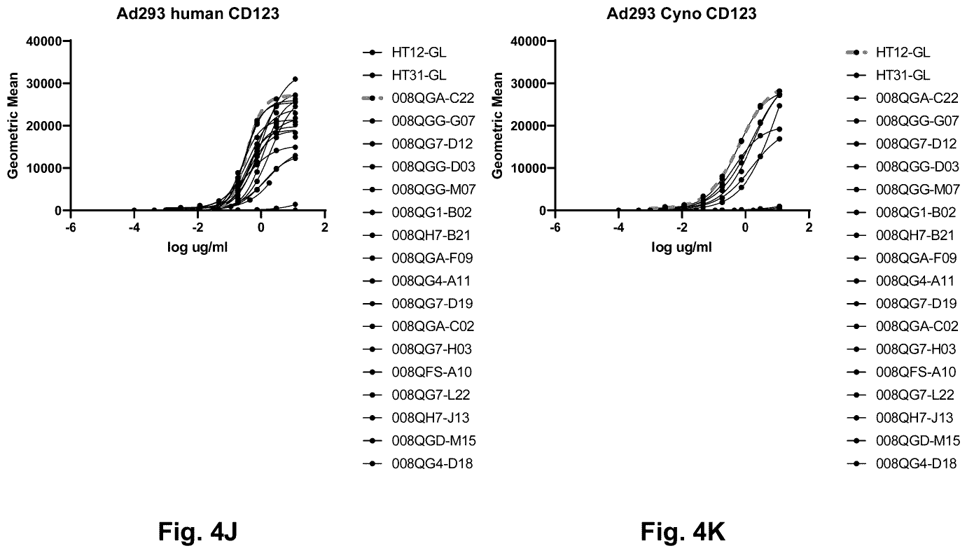

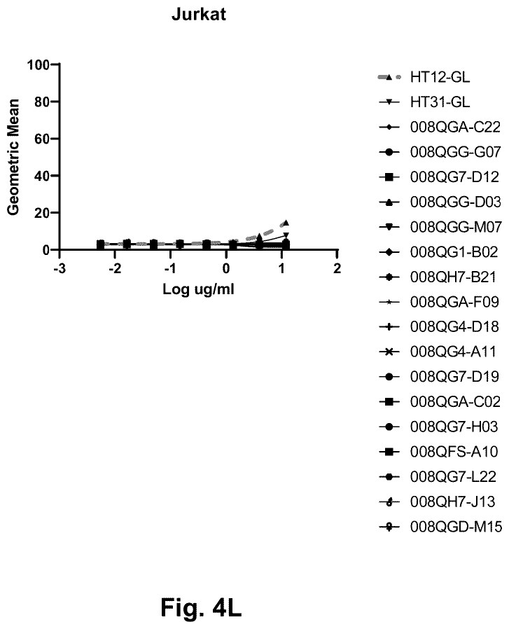

The CD123 antibody HT12-GL binding capacity was high in cell lines transduced with CD123/IL3Rα constructs (AD293cIL3Rα and AD293hIL3Rα) and cells transduced with dox-inducible CD123/IL3Rα constructs and treated with doxycycline (THP-1 inducible+Dox) compared to other cell lines tested (FIG. 4A). The CD123 antibody HT12-GL bound human and cynomolgus IL3Rα expressed on 293 cells, but not rat or mouse IL3Rα (FIG. 4B).

CD123 antibody HT12-GL binding capacity was assessed across AML cell lines. Mv411 cells, KG-1 cells, OCI-AML5 and EOL-1 cells were maintained in their respective media and were cultured in a humidified atmosphere of 5% CO₂ and at a temperature of 37° C. On the day of plating, cells were spun down at 1500 rpm for 3 minutes, counted, and resuspended to a final concentration of 1×10⁶ cells/ml. Two hundred μl of cells were plated to wells of 96 well microtiter plates. The plate were spun down at 1500 rpm for 3 minutes and flicked to remove media. One hundred μl of FACS buffer (PBS+2% FBS) were added to the wells. Plates were then spun again as above followed by addition of HT12-GL primary antibody in 100 μl of FACS buffer. Antibody concentration started at 10 μg/ml followed by six 1:3 dilutions (no antibody control was also run). Plates were incubated at 4° C. for 30 minutes. Following incubation plates were washed 3 times by spinning down at 1500 rpm for 3 minutes, flicked and 100 μl of FACS buffer were added. Alexa 647 secondary antibody (goat anti-human IgG (H+L)) was added at 1:1000 dilution in 100 μl followed by a 30 minute incubation at 4° C. Plates were washed as above. Following a final wash 100 μl of FACS buffer containing live/dead DAPI (diamidino-2-phenylindole) stain was added to the plates. Cells were analyzed on a BD Bioscience FACSSYMPHONYA3 by collecting 10000 cells. Flow data was analyzed in FlowJo software.

HT12-GL antibody bound EOL-1 cells compared to NIP228 antibodies (FIG. 4C). Various HT antibody clones bound 293 cells expressing human IL3Rα (FIG. 4E) and cynomolgus IL3Rα (FIG. 4F), but not parental 293 cells (FIG. 4D). Various HT antibody clones also bound Mv4-11 cells (FIG. 4G), OCI-AML5 cells (FIG. 4H), but not IL3Rα-negative Jurkat cells (FIG. 4I). HT antibody clones and various 008Q antibody clones bound 293 cells expressing human IL3Rα (FIG. 4J) and cynomolgus IL3Rα (FIG. 4K), but not IL3Rα-negative Jurkat cells (FIG. 4L).

THP-1 cells were transfected with an IL3Rα expression construct under the control of a doxycycline inducible promoter. THP-1 cells were maintained in DMEM containing 10% heat-inactivated fetal bovine serum (FBS) and were cultured in a humidified atmosphere of 5% CO₂ and at a temperature of 37° C. Doxorubicin (Dox) inducible cells were stimulated with 1 μg/ml dox overnight before use. Under these conditions, the THP-1 cells were high CD123 expressor cells. On the day of plating, cells were spun down at 1500 rpm for 3 minutes, counted, and resuspended to a final concentration of 1×10⁶ cells/ml. Two hundred μl of cells were plated in wells of 96 well microtiter plates. The plates were spun down at 1500 rpm for 3 minutes and flicked to remove media. One hundred μl of FACS buffer (PBS+2% FBS) was added to each well. The plates were spun again as above followed by addition of primary antibody HT12-GL or HT12-GL ADC in 100 μl of FACS buffer. The antibody concentration started at 12 μg/ml followed by six 1:3 dilutions (no antibody control was also run). The plates were incubated at 4° C. for 30 minutes. Following incubation, the plates were washed 3 times by spinning down at 1500 rpm for 3 minutes, flicked and 100 μl of FACs buffer was added. Alexa 647 secondary antibody (goat anti-human IgG (H+L)) was added at 1:1000 dilution in 100 μl followed by 30 minute 4° C. incubation. The plates were washed as above. Following the final wash 100 μl of FACS buffer containing live/dead DAPI (diamidino-2-phenylindole) stain was added to the plates. Cells were analyzed on a BD Bioscience LSRII by collecting 10,000 cells. Flow data was analyzed in FlowJo software.

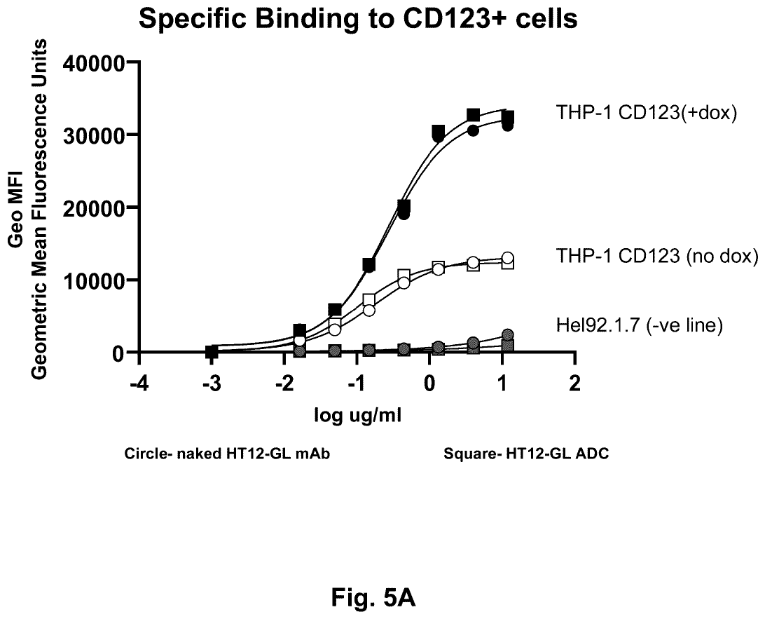

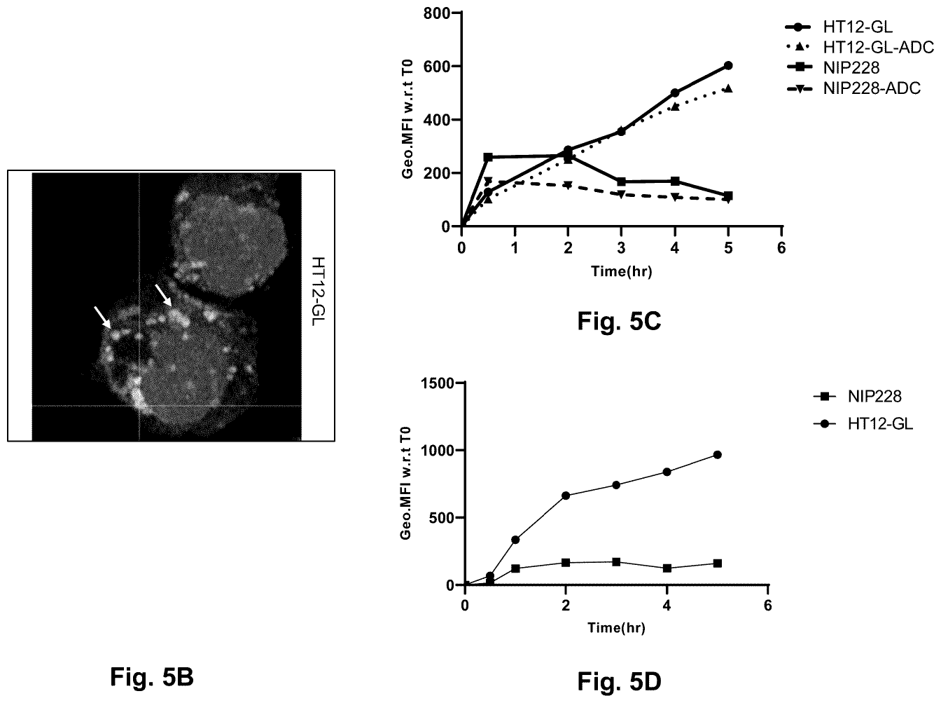

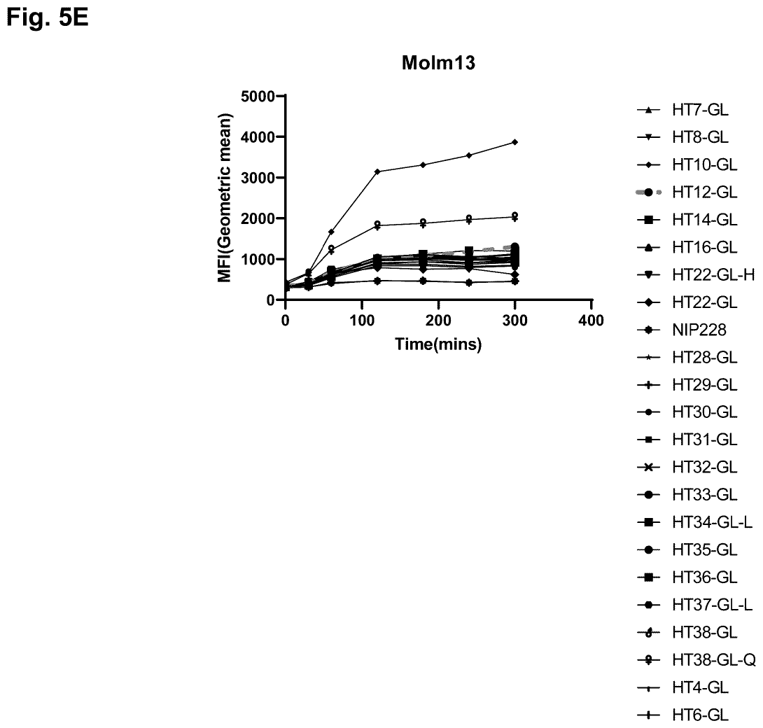

Dox treated THP-1 cells bound HT12-GL antibody and HT12-GL ADC efficiently, while THP-1 cells not treated with Dox bound less HT12-GL antibody and HT12-GL ADC and no binding was observed in control cells (Hel92.1.7 −ve cells) (FIG. 5A).

### Example 6—CD123 Antibody Internalization

CD123 antibody internalization was assessed by immunofluorescence microscopy using EEA1 endosomal marker antibodies.

THP-1 cells expressing Dox inducible CD123 (IL3Rα) were induced by 1 μg/ml doxycycline overnight to overexpress IL3Rα. Cells were incubated with 10 μg/ml of HT12 antibody on ice to allow binding following by incubation at 37° C. for 1 hr to allow internalization. Upon internalization, cells were fixed with 4% formaldehyde and cells were stained with mouse anti EEA1 antibody followed by secondary staining with Alexa Flour 488 goat anti mouse antibody and 546 goat anti human antibody. Cells were assessed on a Zeiss 880 Confocal microscope at 60× magnification. Colocalization of internalized HT12 antibody and early endosome marker EEA1 showed localization of HT12 antibodies in early endosomes 1 hour after addition of HT12 antibodies (FIG. 5B, white puncta).

Internalization was also measured by conjugating a pH sensitive anti human Fc Deep Red Dye with either HT12-GL antibody, HT12-GL ADC (HT12-GL-SG3932), Isotype control NIP228 antibody, or control NIP228 ADC. This dye fluoresces only when it reaches the lysosomes upon internalization. Cells were allowed to internalize 10 μg/ml antibodies and ADCs for 5 hours and fluorescence signal was measured by Flow at the end of each time point. Each internalization time point represents the geometric mean of signal subtracted from TO time point. HT12-GL antibody and HT12-GL ADC internalized efficiently into dox treated THP-1 cells (FIGS. 5C and 5D).

Internalization of various HT antibody clones was also tested in Molm13 cells (FIG. 5E).

### Example 7—CD123 Antibody Inhibits IL3-Mediated Signaling and Proliferation

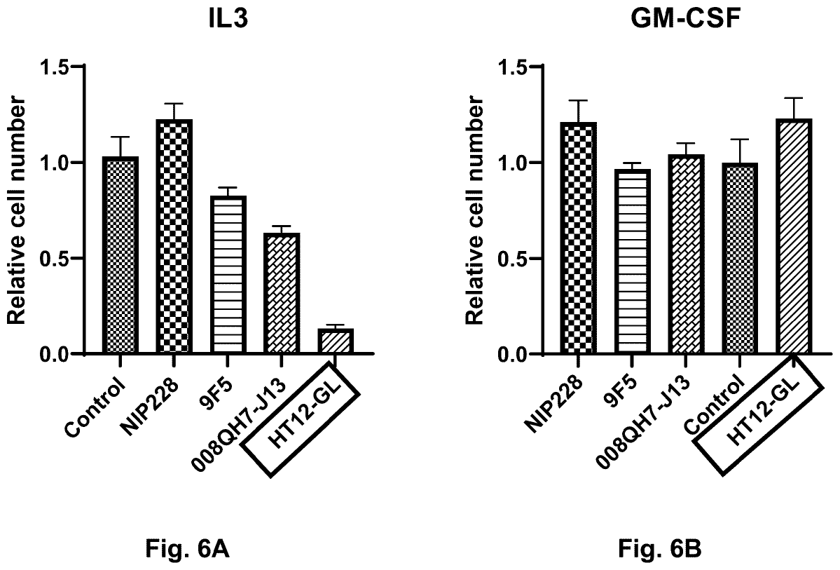

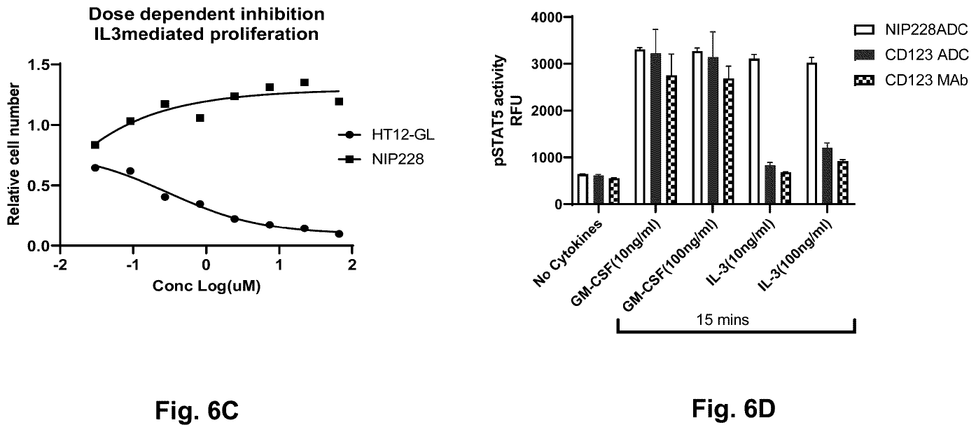

The erythroleukemia cell line TF-1 that can proliferate only in the presence of one of the following growth factors, either IL-3 or GM-CSF was used. TF-1 cells were washed with RPMI and cultured overnight in the complete RPMI media (RPMI-1640, 10% fetal bovine serum) without any growth factor. To block Fc receptors on the cell surface, cells were incubated with human Fc block at RT for 5-10 mins. Cells were spun at 1300 rpm for 5 mins and were plated at 8,000 cells per well in complete media in either the presence or absence of 10 μg/ml of anti CD123 antibodies (HT12 and J13) or control antibodies (NIP228, 9F5). A growth factor, either IL-3 (1 ng/ml) or GM-CSF (2 ng/ml), was added to the cells to initiate proliferation. Cells were incubated at 37° C. in a humidified 5% CO₂ incubator for 3 days. Cell viability was measured by CellTiter-Glo® (CTG) Luminescent cell Viability Assay. CTG reagent was added into the wells at equal volume and the plates were shaken at room temperature for 10 minutes. Then the absorbance was measured by Envision luminescence reader. The relative cell number in each well was calculated by dividing the value of each sample treated with an antibody by the average values of wells with the untreated cells (control). The 9F5 control antibody did not inhibit IL-3 dependent proliferation. HT12-GL antibodies, and to a lesser extent J13 antibodies, inhibited IL-3 dependent proliferation of TF-1 (FIG. 6A) but not GM-CSF mediated proliferation (FIG. 6B). Isotype antibody had no effect on cell proliferation.

To measure the concentration of antibody needed for IL3 inhibition, TF-1 cells were seeded at 8,000 cells per well and antibodies were added at a 10 point dilution starting at 10 μg/ml. Then IL-3 was added at a concentration of 1 ng/ml. The final antibody concentration ranged from 66 M to 0.03 M. Cells were incubated at 37° C. for 3 days. The relative cell number in each well was determined by Cell Titre Glo as described above. The relative cell number plotted against the antibody concentration showed that HT12-GL antibodies dose dependently inhibited TH-1 cell proliferation in the presence of IL-3 (FIG. 6C).

The effect of CD123 antibodies and CD123 ADC (HT12-GL-SG3932) on the IL3 mediated JAK-STAT5 signaling pathway was measured. TF-1 cells were cultured in RPMI and cultured in complete RPMI media (RPMI-1640, 10% FBS) with 2 ng/ml GM-CSF. Cells were washed in Stem Span H3000 Serum Free media and treated with isotype ADC, CD123 ADC (HT12-GL-SG3932), or CD123 antibody (premade in Stem Span H3000 SFM media) for 2 hrs at 37° C. in an incubator. Cells were washed and plated in 96 well U bottom plate at 2,000 cells per well density. Cells were stimulated with either 10 ng/ml or 100 ng/ml of GM-CSF or IL3 for 15 minutes or left untreated (no cytokines). After the stimulation period, cells were fixed immediately by adding prewarmed BD Cytofix Fixation buffer in the same well. Fixation was done at 37° C. for 12 minutes. Following fixation, cells were permeabilized by adding chilled Perm Buffer III on ice for 30 minutes. Cells were washed with Stain buffer and stained with pStat5-Bv421 antibody overnight at 4° C. pSTAT5 activation was measured by Flow Cytometry analysis. Geometric mean of live cells positive for pSTAT5 was plotted as pSTAT5 activity RFU. Results showed that both GM-CSF and IL3 can increase pSTAT5 activation upon stimulation for 15 mins when pretreated with isotype ADC alone. Consistent with the earlier findings, both the CD123 antibody and CD123 ADC (HT12-GL-SG3932) inhibited STAT5 signaling pathway mediated by IL3 and not GM-CSF (FIG. 6D).

### Example 8—CD123 ADC Mediated Cytotoxicity

THP-1 cells expressing CD123 under the control of a dox inducible promoter were grown in the presence of 1 μg/ml doxycycline. High CD123 expressor THP-1 cells and non-CD123-expressing Hel92.1.7 cells were plated at 2500 cells in 30 μl per well in 384 well plates for 24, 48, 72 and 144 hours in duplicates for each time point. The cells were treated with either HT12-GL ADC (HT12-GL-SG3932) or isotype NTP228 ADC. The drug treatments were prepared at 240 μg/ml and serially diluted 1:4. Ten μl of treatment was added in triplicate to achieve 60 μg/ml to 0.00001 μg/ml in treatment for all plates and time points. Baseline was established treating with complete growth medium without ADCs. The plates for each time point were read for cytotoxicity and caspase activity using CellTiter-Glo® (CTG) and Caspase Glo® 3/7 reagents from Promega as recommended by vendor. The data was normalized to baseline.

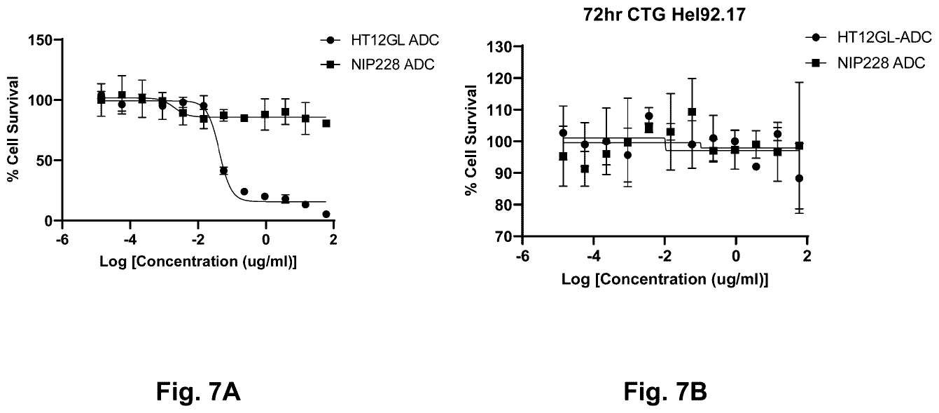

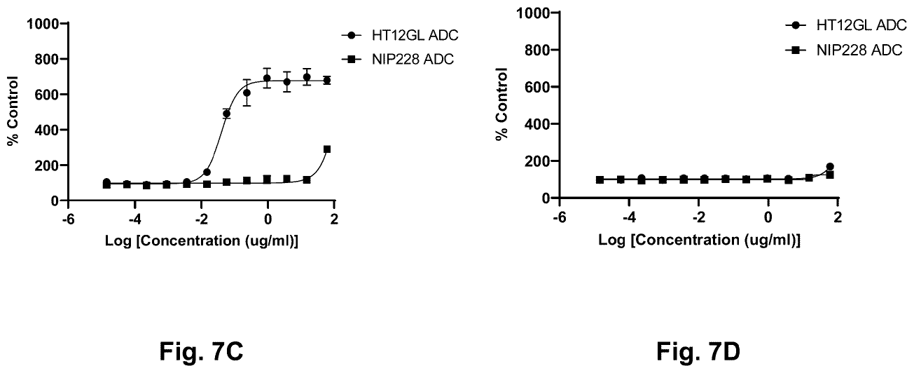

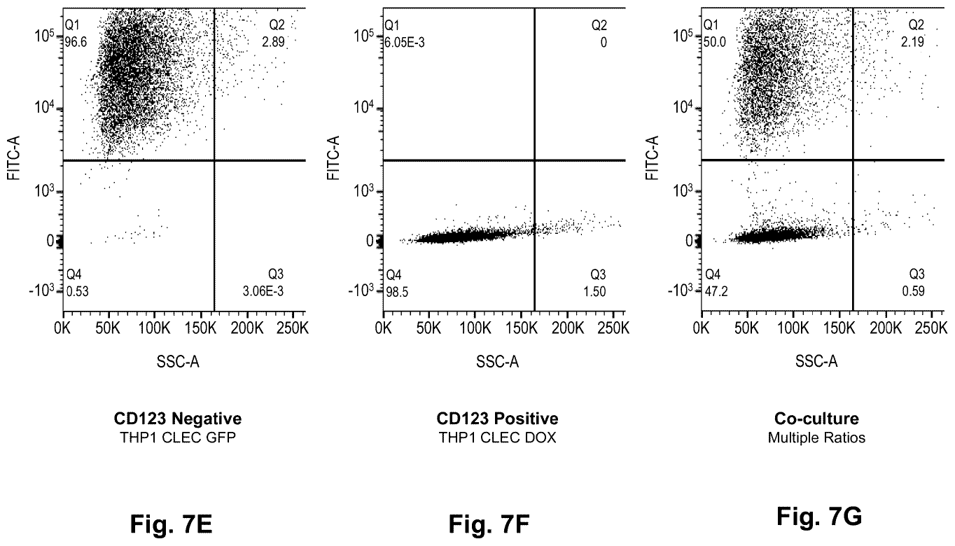

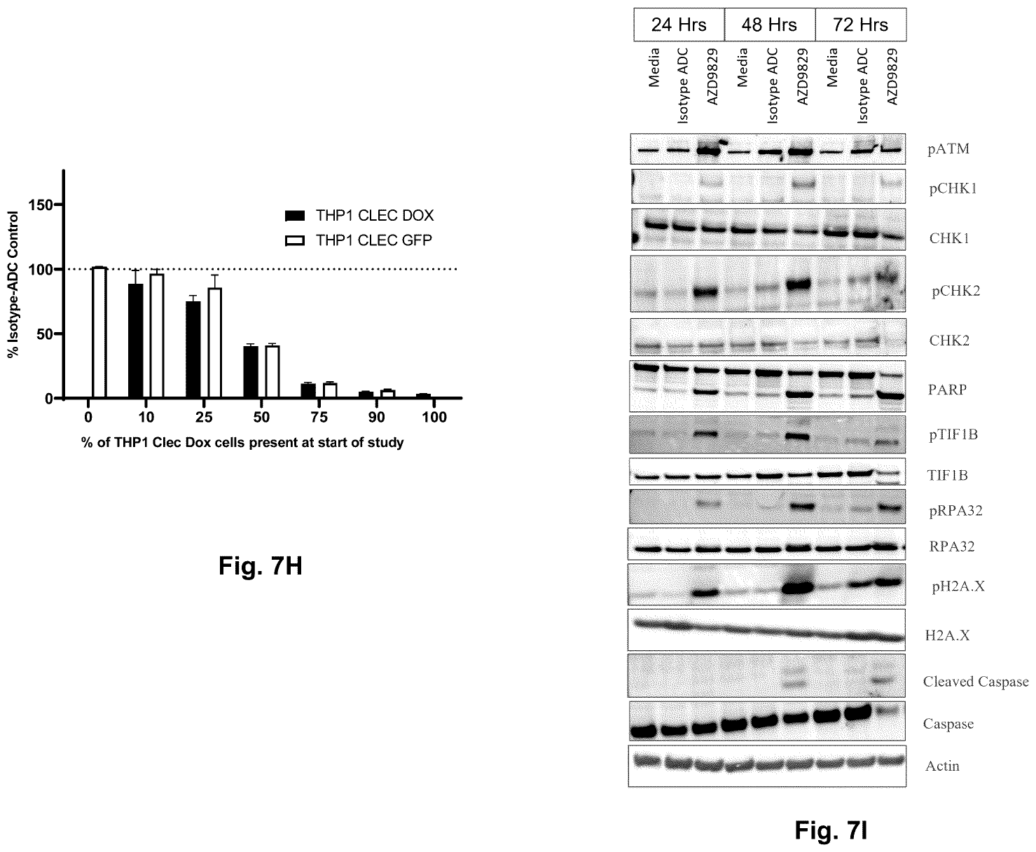

HT12-GL ADC (HT12-GL-SG3932) showed effective killing of CD123+dox THP-1 cells (FIG. 7A), but not CD123-negative Hel92.1.7 cells (FIG. 7B). Furthermore, HT12-GL ADC (HT12-GL-SG3932) showed increased caspase 3/7 activity in CD123-positive dox THP-1 cells (FIG. 7C), but not CD123-negative Hel92.1.7 cells (FIG. 7D).

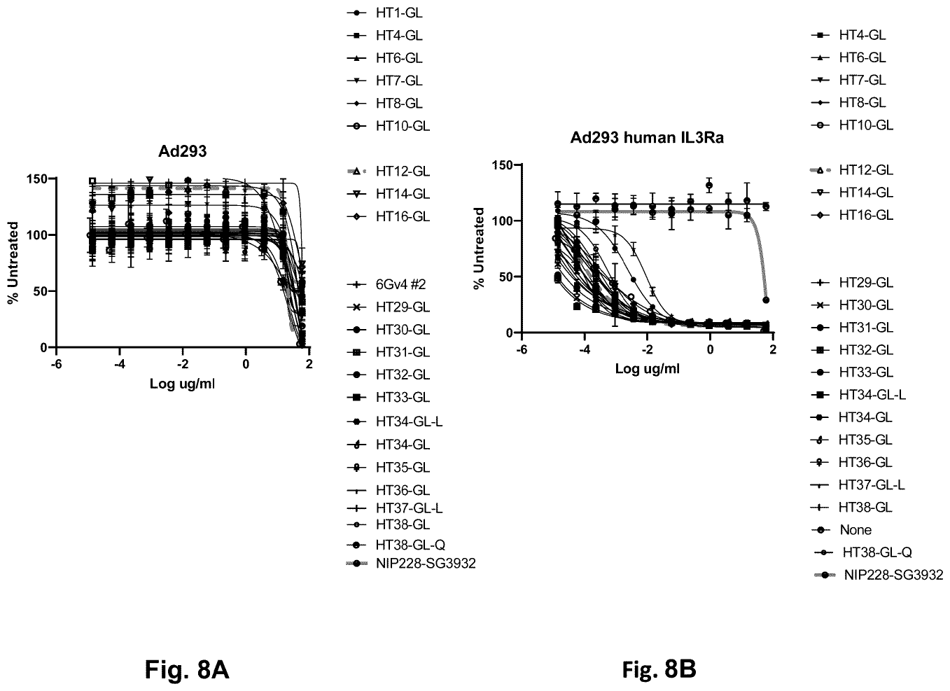

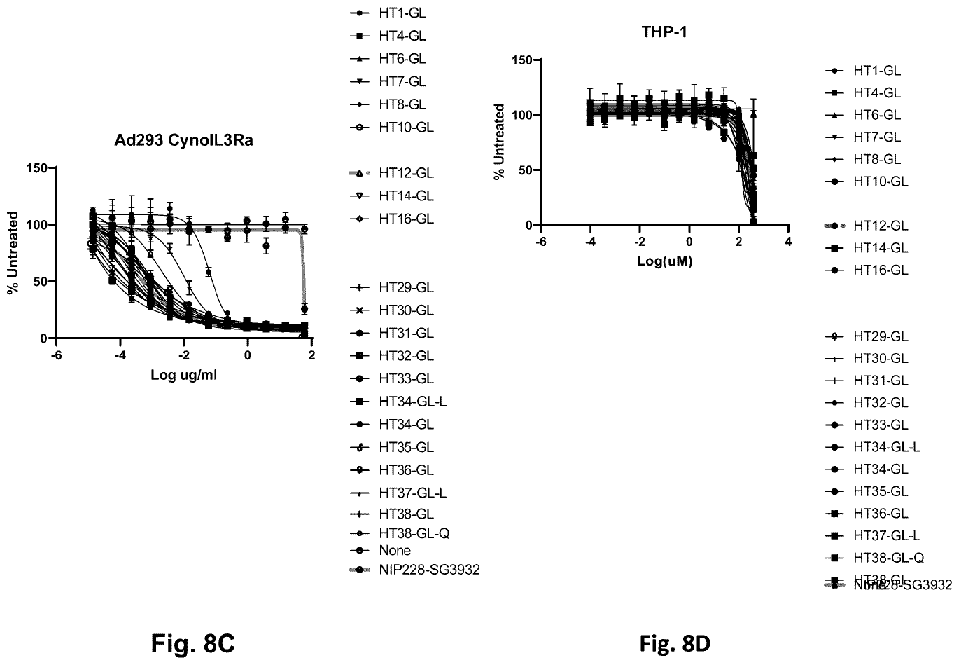

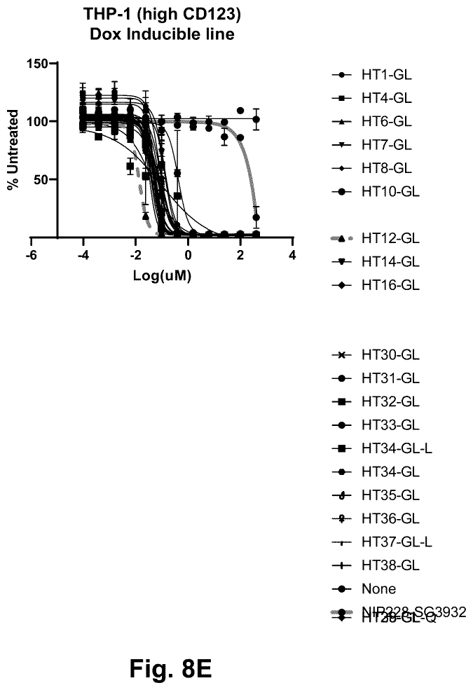

Cell killing was also tested for various HT-SG3932 antibody clones. While toxicity in 293 cells of all tested HT antibody clone ADC was only observed at the highest tested concentrations (FIG. 8A), cytotoxicity was observed for the various HT antibody clone ADC at lower concentrations in human IL3Rα expressing 293 cells (FIG. 8B) and cynomolgus IL3Rα expressing 293 cells (FIG. 8C). Furthermore, HT antibody clone ADC (HT-SG3932) did show toxicity only at the highest tested concentrations in IL3Rα-negative THP-1 cells (FIG. 8D), but showed cytotoxicity at lower concentrations in IL3Rα expressing dox-treated THP-1 cells (FIG. 8E).

### Example 9A—CD123 Antibody Drug Conjugates Induce Bystander Killing

THP-1 cells were maintained in DMEM media containing 10% heat-inactivated FBS and were cultured in a humidified atmosphere of 5% CO₂ and at a temperature of 37° C. Cells were stimulated with 1 μg/ml dox overnight before use or maintained without dox. Unstimulated cells contained GFP marker. Cells were plated in ratio of stimulated to unstimulated cells in 24 well plates at 100/0, 90/10, 75/25, 50/50, 25/75, 10/90 and 0/100% respectively. Two hundred fifty μl of cells at 10×10⁴ cells/ml were added to wells along with 250 μl of media. HT12-GL ADC (HT12-GL-SG3932) or NIP228 (isotype control; NIP228-SG3932) ADCs was added to wells to a final concentration of 100 ng/ml. Plates were incubated as above for 72 hours. Following incubation all cells/media were transferred to deep well 96 well plates. Plates were spun down at 1500 rpm for 3 minutes followed by flick to remove media. One hundred μl of FACS buffer (PBS+2% FBS) was added to the plates and cells resuspended. The cells were then transferred to microtiter plates. Plates were spun down at 1500 rpm for 3 minutes and flicked to remove FACS buffer. Following a final wash 100 μl of FACS buffer containing live/dead DAPI (diamidino-2-phenylindole) stain was added to the plates. Cells were analyzed on a BD Bioscience FACSSYMPHONYA3 by collecting 90 μl of the sample. All cells were counted from the 90 μl. Flow data was analyzed in FlowJo software.

While HT12-GL ADC (HT12-GL-SG3932) did not affect the viability of CD123 negative THP-1 CLEC GFP cells (FIG. 7E), it did kill CD123-positive THP-1 CLEC DOX cells (FIG. 7F). Co-cultures of CD123-negative and CD123-positive THP-1 cells resulted in bystander killing of CD123-negative cells (FIG. 7G). Furthermore, increasing the ratio of CD123-positive dox THP-1 cells increased the bystander killing of CD123-negative THP-1 cells (FIG. 7H).

### Example 9B—CD123 Antibody Drug Conjugates Induce Double Strand Breaks

THP1 cells were maintained in RPMI containing 10% heat-inactivated FBS and were cultured in a humidified atmosphere of 5% CO2 and at a temperature of 37° C. Doxycyclin inducible cells were stimulated with 1 μg/ml dox overnight before use. On the day of plating, cells were spun down at 1500 rpm for 3 minutes, counted, and resuspended to a final concentration of 3×10⁶ cells/ml. One ml of cells were plated in 6 well plates along with 4 ml of media. HT12-GL ADC (e.g., HT12-GL-SG3932) or NIP228-SG3932 (isotype) ADCs were added to cells at a final concentration of 0.05 μg/ml. Cells were incubated with ADCs for 24, 48 and 72 hours. Cells were collected at appropriate time points, spun down at 1500 rpm for 3 minutes. Cells were then lysed with 500 μl of MPER (Mammalian Protein Extraction Reagent) containing Halt protease & phosphatase inhibitor. Lysates were stored at −80° C. until western blot was performed.

Lysates were thawed and BCA was performed to determine protein concentrations. Protein concentrations were adjusted and ˜10 μg of protein was loaded to gels. Gels were run until appropriate molecular weight sizes could be detected. Gels were transferred to nitrocellulose membranes with Iblot system. Blots were blocked with SuperBlock for a minimum of 1 hour. Primary antibody (see list below) was added at 1:1000 dilution to blocking buffer except Actin loading control (1:10000 dilution) and incubated at 4° C. with rocking overnight. Blots were then washed and secondary HRP labeled antibody was added in superblock for 1 hour at room temperature. Following incubation, blots were washed and SuperSignal West Coast Pico was added to the blots. Blots were imaged on ImageQuant 4000 imager. Primary antibodies were pRPA Cell Signaling 4124, pTIF1B Cell Signaling 4127S, pCHK2 Cell Signaling 2197, pCHK1 Cell Signaling 12302S, cCaspase Cell Signaling 9662S, PARP Cell Signaling 9542, and Actin Sigma A1978.

Treatment with CD123 ADC (HT12-GL-SG3932) induced in increase in double strand break related proteins (FIG. 7I).

### Example 10—Potency of CD123 Antibody Drug Conjugate Compared to CD33 Mylotarg ADC

The potency of HT12-GL ADC (HT12-GL-SG3932) was compared to CD33 Mylotarg ADC and isotype ADC in colony forming unit (CFU) assays. For colony forming unit assays, primary healthy donor samples and AML patient samples were subjected to a dose titration of HT12-GL ADC (HT12-GL-SG3932), Isotype ADC, or Mylotarg for 24 hours. Cells were subsequently mixed in Methocult (StemCell) and seeded in 6-well SmartDish's (StemCell). Colonies were counted at 14 days post-seeding using a STEMvision instrument (StemCell). EC50 values were calculated based on the reduction of colonies following drug treatment compared to control groups.

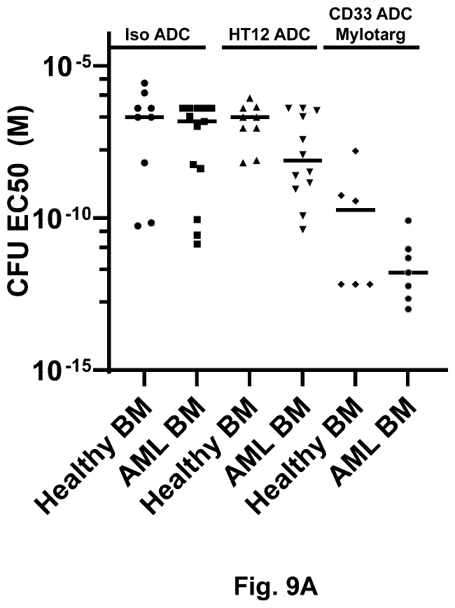

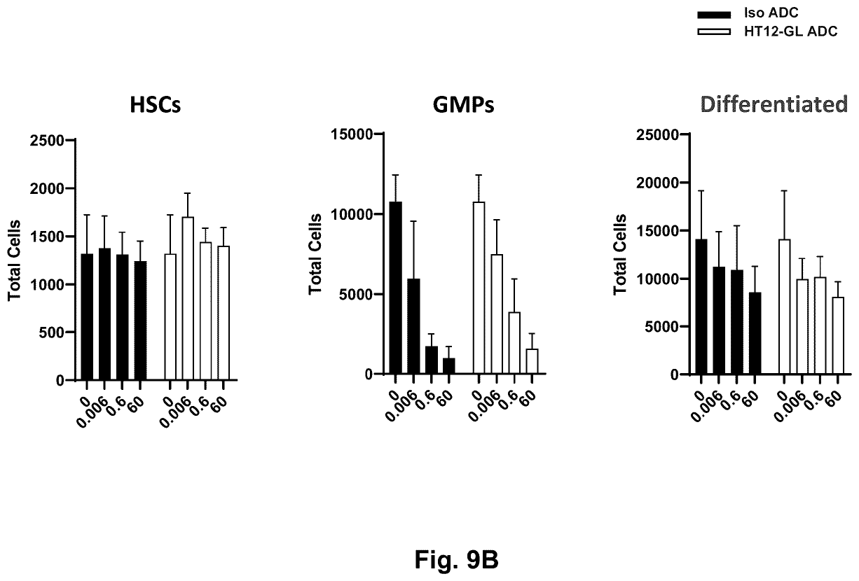

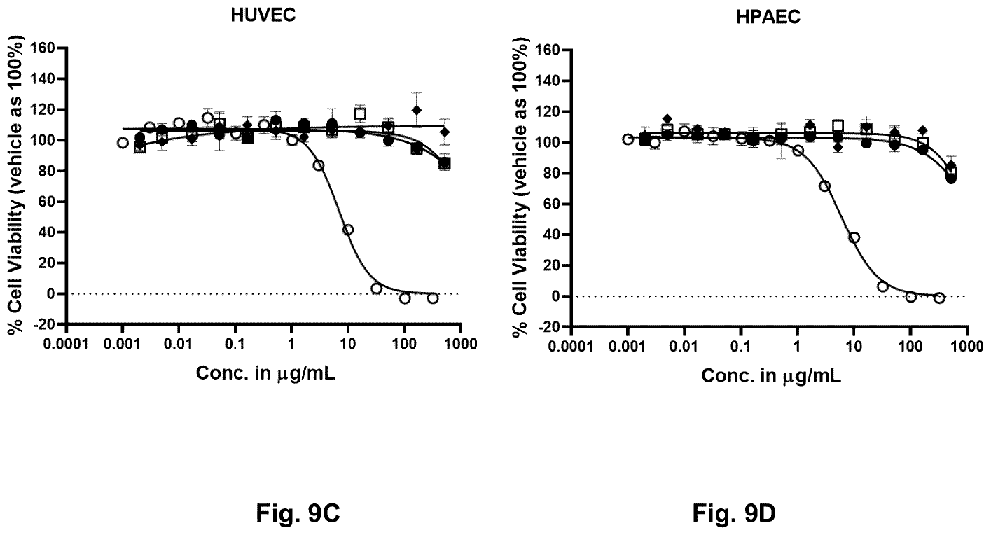

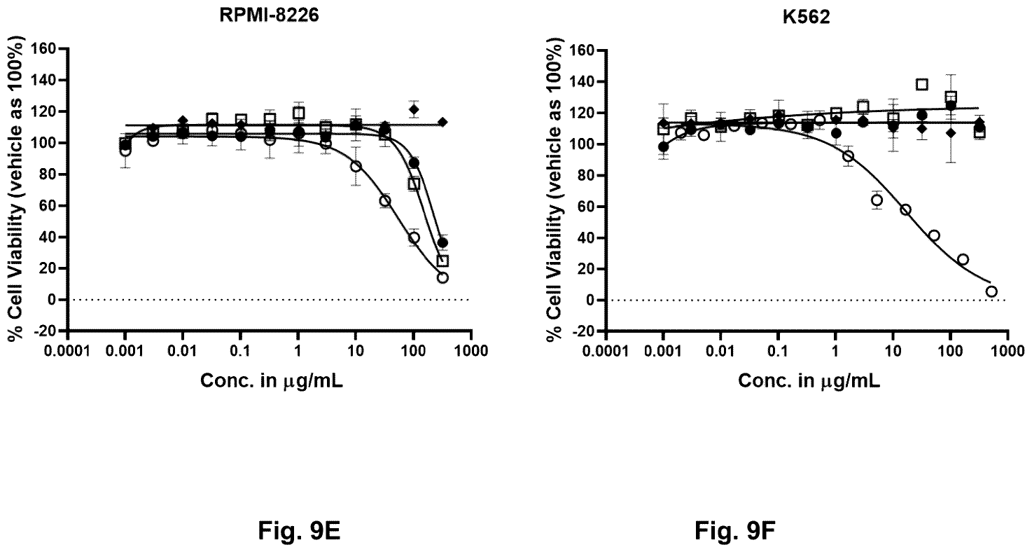

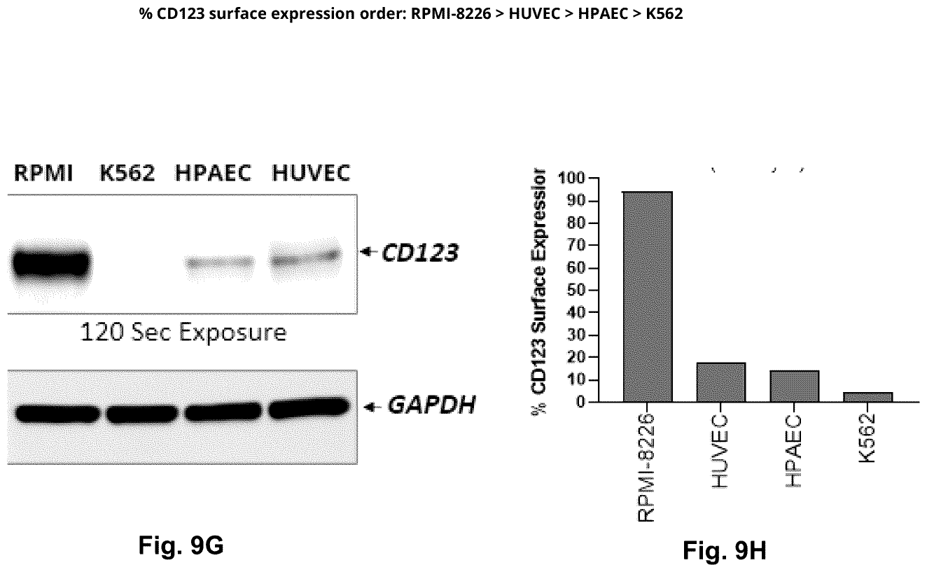

HT12-GL ADC (HT12-GL-SG3932) showed similar potency as CD33 Mylotarg ADC in reducing CFU numbers in AML BMMCs compared to healthy BMMCs, but HT12-GL ADC (HT12-GL-SG3932) lacked CFU toxicity in healthy donor BMMC, while Mylotarg was toxic in healthy BMMC (FIG. 9A).

### Example 11—CD123 ADC Lacks Cytotoxicity Towards HSC, GMP, and Differentiated Cells in CD34+ BMMC Expansion Assays

For CD34⁺ BMMC expansion assays, healthy donor BMMCs were seeded into 24-well plates containing a dose titration of HT12-GL ADC (HT12-GL-SG3932) or isotype ADC along with StemSpan SFEM II (StemCell) supplemented with CD34⁺ cell expansion supplement (StemCell), 1 M UM729 (StemCell), and 500 nM Stemregenin (StemCell). Cells were cultured for 6 days and subsequently subjected to immunophenotypic analysis using flow cytometry. Retention of healthy donor HSCs (Lineage⁻ CD34⁺ CD38⁻ CD90⁺ CD45RA⁻), GMPs (GMP: Lineage⁻ CD34⁺ CD38⁺ CD135⁺ CD45RA⁺) and Differentiated cells (Lineage⁺) were bench marked against control wells.

HT12-GL ADC (HT12-GL-SG3932) had no cytotoxic effects on HSC, GMP or differentiated cells as compared to isotype ADC (FIG. 9B).

### Example 12—CD123 ADC Lacks Cytotoxicity Towards CD123-Expressing HUVEC and HPAEC

The CD123-expressing endothelial cell lines HUVEC and HPAEC and control cell lines RPMI-8226 (high CD123 expressing) and K562 (low expressing or negative for CD123) were cultured and co-incubated with Comparator-1 ADC, NIP228-SG3932 (isotype control for HT12-GL-SG3932) HT12-GL-SG3932, or CD123 mAb (HT12-GL) for 3 days at concentrations ranging from 0.0017 to 520 μg/mL for CD123 mAb, HT12-GL-SG3932, or NIP228-SG3932) or 0.0010 to 320 μg/mL for IMGN-IGN). Cell viability was assessed by CellTiter-Glo Cell Viability Assay and IC₅₀ for viability was calculated (when possible) for each compound for each cell line. Expression of CD123 was assessed by western blot surface expression or by flow cytometry (FIG. 9C-9H).

Results are shown in Table 3. HT12-ADC did not demonstrate enhanced cytotoxicity against CD123 expressing endothelial cell lines compared to isotype control. Comparator-1 ADC showed higher cytotoxicity against all cell lines compared to HT12-GL-SG3932 which was not CD123 dependent.

TABLE 3
Cytotoxicity of HT12-GL TOPO ADC (e.g., HT12-
GL-SG3932) Against CD123 Expressing Endothelial
Cell Lines Compared to Comparator-1 ADC

| | Cytotoxicity (Viability IC50 μg/mL) | | | |
|---|---|---|---|---|
| Compound | HUVEC | HPAEC | RPMI-8226 | K562 |
| Comparator-1 ADC | 6.9 | 6.0 | 53.9 | 10.3 |
| NIP228-SG3932 | >520 | >520 | 248 | >520 |
| HT12-GL-SG3932 | >520 | >520 | 374 | >520 |
| CD123 mAb | >520 | >520 | >520 | >520 |
| Comparator-1 | >75x | >87x | 7x | >50x |
| ADC/HT12-GL- | | | | |
| SG3932 | | | | |

Comparator-1 comprises a CD123-ADC of pivekimab sunirine, which is described in U.S. Pat. Nos. 10,919,969 and 11,332,535. In mice bearing subcutaneous EOL-1 tumors, Comparator-1 ADC induced complete tumor regression at 0.24 mg/kg at a single dose, which is consistent with published data. See Kovtun et al., Blood Adv (2018) 2(8):848-858.

### Example 13—In Vivo Subcutaneous Xenograft Studies

Mv4-11 cells that express about 48,000 CD123/IL3Rα per cell were maintained in the IMDM (Iscove's Modified Dulbecco's Medium) containing 10% heat-inactivated FBS and were cultured in a humidified atmosphere of 5% CO₂ and at a temperature of 37° C. On the day of implantation, cells were spun down at 1500 rpm for 5 minutes, counted, and resuspended to a final concentration of 25×10⁶ cells/mL in a 1:1 PBS: Matrigel® (Corning) mixture. Female CB-17 SCID mice were each injected subcutaneously in the right flank with 5×10⁶ cells in 200 μL volume.

When average tumor volume reached ˜150-250 mm³ on Day 21, mice were randomized into treatment groups using the Matched Distribution method built into the Study Director software. Group designation, dose levels and number of mice per group are presented in Table 3.

Test and control ADC were diluted from a stock solution with vehicle buffer (20 mM histidine, 240 mM sucrose, 0.02% Polysorbate 80, pH 6.0) and were administered as a single intravenous (IV) injection according to body weight. The group destination and dose levels for the Mv4-11 subcutaneous xenograft study are shown in Table 4.

TABLE 4

| | | Dose | | Route of | Number |
|---|---|---|---|---|---|
| | | level | | administration, | of |
| Group | Treatment | (mg/kg) | | dosing regimen | animals |
| 1 | Untreated | NA | | NA | 6 |
| 2 | NIP-228-SG3932 | 10 | mg/kg | IV, single dose | 3 |
| 3 | NIP-228-SG3932 | 8 | mg/kg | IV, single dose | 3 |
| 4 | NIP-228-SG3932 | 6 | mg/kg | IV, single dose | 3 |
| 5 | NIP-228-SG3932 | 4 | mg/kg | IV, single dose | 3 |
| 6 | NIP-228-SG3932 | 2 | mg/kg | IV, single dose | 3 |
| 7 | NIP-228-SG3932 | 1 | mg/kg | IV, single dose | 3 |
| 8 | NIP-228-SG3932 | 0.5 | mg/kg | IV, single dose | 3 |
| 9 | HT12-GL-SG3932 | 10 | mg/kg | IV, single dose | 3 |
| 10 | HT12-GL-SG3932 | 8 | mg/kg | IV, single dose | 3 |
| 11 | HT12-GL-SG3932 | 6 | mg/kg | IV, single dose | 3 |
| 12 | HT12-GL-SG3932 | 4 | mg/kg | IV, single dose | 3 |
| 13 | HT12-GL-SG3932 | 2 | mg/kg | IV, single dose | 3 |
| 14 | HT12-GL-SG3932 | 1 | mg/kg | IV, single dose | 3 |
| 15 | HT12-GL-SG3932 | 0.5 | mg/kg | IV, single dose | 3 |
| 23 | J13-SG3932 | 10 | mg/kg | IV, single dose | 3 |
| 24 | J13-SG3932 | 8 | mg/kg | IV, single dose | 3 |
| 25 | J13-SG3932 | 6 | mg/kg | IV, single dose | 3 |
| 26 | J13-SG3932 | 4 | mg/kg | IV, single dose | 3 |
| 27 | J13-SG3932 | 2 | mg/kg | IV, single dose | 3 |
| 28 | J13-SG3932 | 1 | mg/kg | IV, single dose | 3 |
| 29 | J13-SG3932 | 0.5 | mg/kg | IV, single dose | 3 |

Tumor volume and body weight were measured 1-2 times weekly from Day 15 to Day 98 after Mv4-11 implant. Tumors were measured by caliper and the volumes of tumors were calculated using the following formula:

Tumor Volume=[length (mm)×width (mm)²]/2; where the length and the width are the longest and shortest diameters of the tumor respectively.

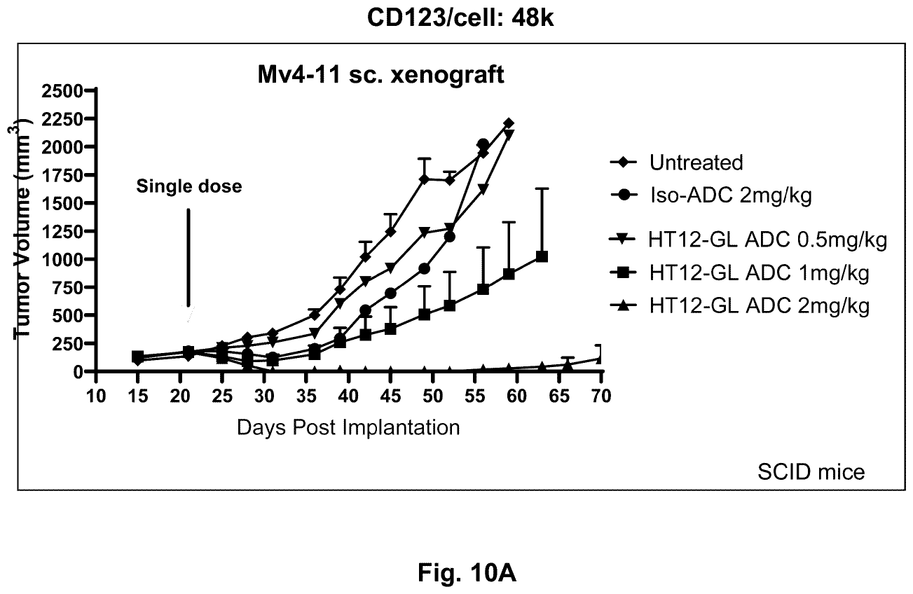

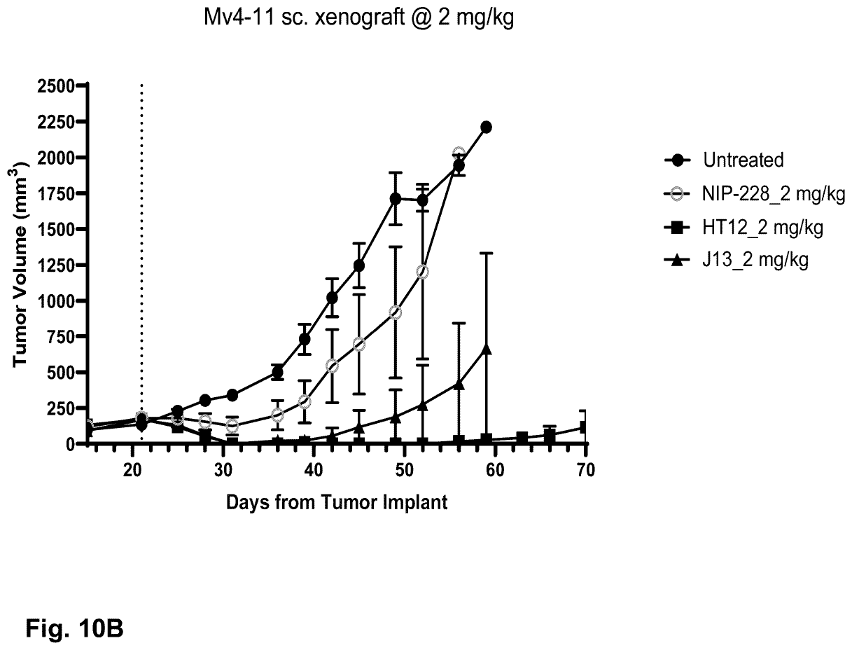

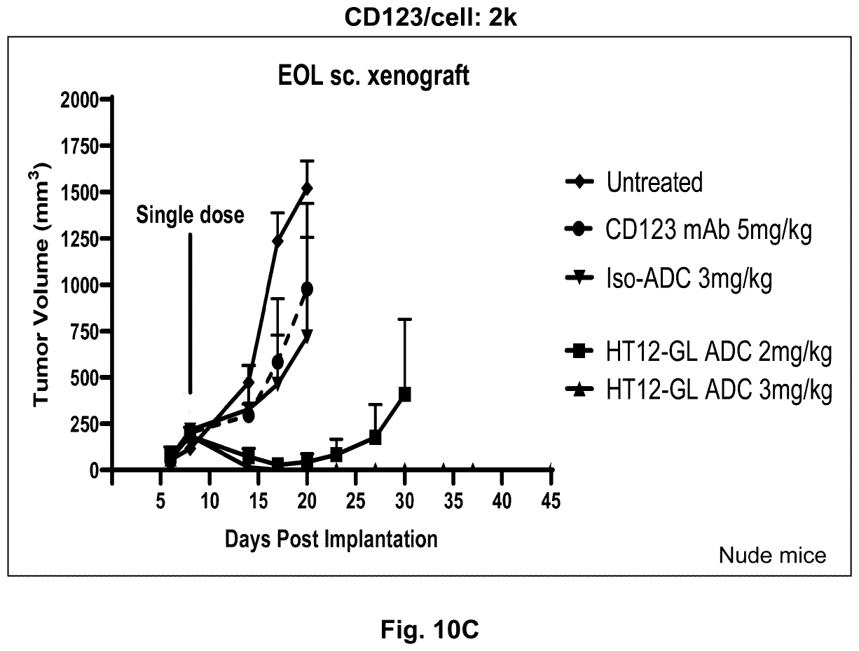

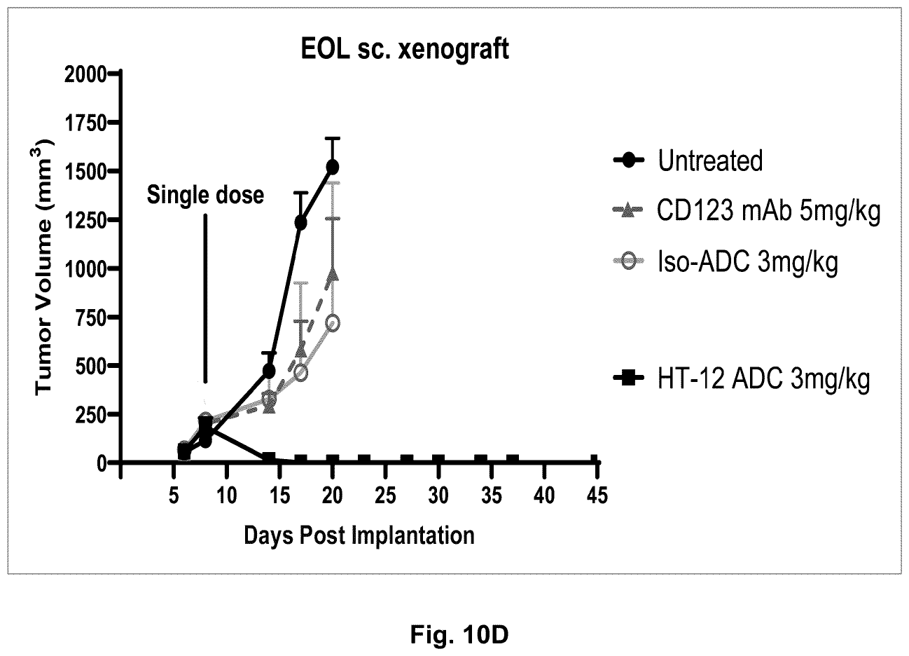

HT12-GL ADC (HT12-GL-SG3932) induced a dose-dependent anti-tumor response at a dose range of 0.5 mg/kg to 2 mg/kg (FIG. 10A). At 2 mg/kg dose level, HT12-GL ADC (HT12-GL-SG3932) induced complete tumor regression, while the control iso ADC (NIP-228-SG3932 ADC) induced minimal anti-tumor effect (FIG. 10A).

At 2 mg/kg dose level, HT12-GL ADC (HT12-GL-SG3932) induced complete tumor regression, and other antibody ADC (J13-SG3932) induced partial tumor growth inhibition, while the control isotype ADC (NIP-228-SG3932) induced minimal anti-tumor effect (FIG. 10B).

EOL-1 cells that express about 2,000 CD123/IL3Rα per cell were maintained in the RPMI-1640 medium containing 10% heat-inactivated FBS and were cultured in a humidified atmosphere of 5% CO₂ and at a temperature of 37° C. On the day of implantation, cells were spun down at 1500 rpm for 5 minutes, counted, and resuspended to a final concentration of 50×10⁶ cells/mL in PBS. Female Nude mice were each injected subcutaneously in the right flank with 10×10⁶ cells in 200 μL volume. When average tumor volume reached ˜150-250 mm³ on Day 9, mice were randomized into treatment groups using the Matched Distribution method built into the Study Director software. Group designation, dose levels and number of mice per group are presented in the following table.

Test and control ADC were diluted from a stock solution with vehicle buffer (20 mM histidine, 240 mM sucrose, 0.02% Polysorbate 80, pH 6.0) and were administered as a single intravenous (IV) injection according to body weight. The group destination and dose levels in the EOL-1 subcutaneous xenograft study are shown in Table 5.

TABLE 5

| | | Dose level | Route of | Number of |
|---|---|---|---|---|
| Group | Treatment | (mg/kg) | administration | animals |
| 1 | Untreated | NA | NA | 6 |
| 2 | NIP-228-SG3932 | 1 mg/kg | IV, single dose | 3 |
| 3 | NIP-228-SG3932 | 2 mg/kg | IV, single dose | 3 |
| 4 | NIP-228-SG3932 | 3 mg/kg | IV, single dose | 3 |
| 5 | NIP-228-SG3932 | 4 mg/kg | IV, single dose | 3 |
| 6 | NIP-228-SG3932 | 5 mg/kg | IV, single dose | 3 |
| 7 | HT12-SG3932 | 1 mg/kg | IV, single dose | 3 |
| 8 | HT12-SG3932 | 2 mg/kg | IV, single dose | 3 |
| 9 | HT12-SG3932 | 3 mg/kg | IV, single dose | 3 |
| 10 | HT12-SG3932 | 4 mg/kg | IV, single dose | 3 |
| 11 | HT12-SG3932 | 5 mg/kg | IV, single dose | 3 |
| 12 | HT12 mAb | 5 mg/kg | IV, single dose | 5 |

Tumor volume and body weight were measured twice weekly from Day 6 to Day 60 after EOL-1 implant. Tumors were measured by caliper and the volumes of tumors were calculated using the following formula:

Tumor Volume=[length (mm)×width (mm)²]/2; where the length and the width are the longest and shortest diameters of the tumor respectively.

HT12-GL ADC induced complete tumor regression at 3 mg/kg and partial tumor growth inhibition at 2 mg/kg, while the control iso ADC (NIP-228 ADC) and naked antibody HT12-GL mAb induced minimal anti-tumor effect (FIGS. 10C and 10D).

### Example 14—In Vivo Disseminated Xenograft Studies

Mv4-11 cells were maintained in the IMDM (Iscove's Modified Dulbecco's Medium) containing 10% heat-inactivated FBS and were cultured in a humidified atmosphere of 5% CO₂ and at a temperature of 37° C. On the day of implantation, cells were spun down at 1500 rpm for 5 minutes, counted, and resuspended to a final concentration of 25×10⁶ cells/mL in PBS. Female NSG mice were each injected intravenously in the tail vein with 5×10⁶ cells in 200 μL volume.

Body weight was measured at day 5 after Mv4-11 cell implant and 4 days after Molm-13 cell implant. Mice were randomized based on body weight into treatment groups using the Matched Distribution method built into the Study Director software. Group designation, dose levels and number of mice per group are presented in the following table.

Test and control articles were diluted from a stock solution with vehicle buffer (20 mM histidine, 240 mM sucrose, 0.02% Polysorbate 80, pH 6.0) and were administered as a single intravenous (IV) injection according to body weight. The group destination and dose levels in Mv4-11 disseminated survival studies are shown in Table 6.

TABLE 6

| | | Dose level | | Route of | Number of |
|---|---|---|---|---|---|
| Group | Treatment | (mg/kg) | | administration | animals |
| 1 | Untreated | NA | | NA | 10 |
| 2 | NIP-228-SG3932 | 10 | mg/kg | IV, single dose | 10 |
| 3 | NIP-228-SG3932 | 5 | mg/kg | IV, single dose | 10 |
| 4 | NIP-228-SG3932 | 2.5 | mg/kg | IV, single dose | 10 |
| 5 | HT12-GL-SG3932 | 10 | mg/kg | IV, single dose | 10 |
| 6 | HT12-GL-SG3932 | 5 | mg/kg | IV, single dose | 10 |
| 7 | HT12-GL-SG3932 | 2.5 | mg/kg | IV, single dose | 10 |
| 11 | J13-SG3932 | 10 | mg/kg | IV, single dose | 10 |
| 12 | J13-SG3932 | 5 | mg/kg | IV, single dose | 10 |
| 13 | J13-SG3932 | 2.5 | mg/kg | IV, single dose | 10 |

Mice were monitored for signs of rough skin/fur and weak hind limbs. When the mice showed hind limb paralysis or incapability of reaching food/water, they were euthanized immediately and the survival days were recorded. Weibull regression was fitted to the survival times, and hazard rate (better survival probability) and median survival were used to compare between groups.

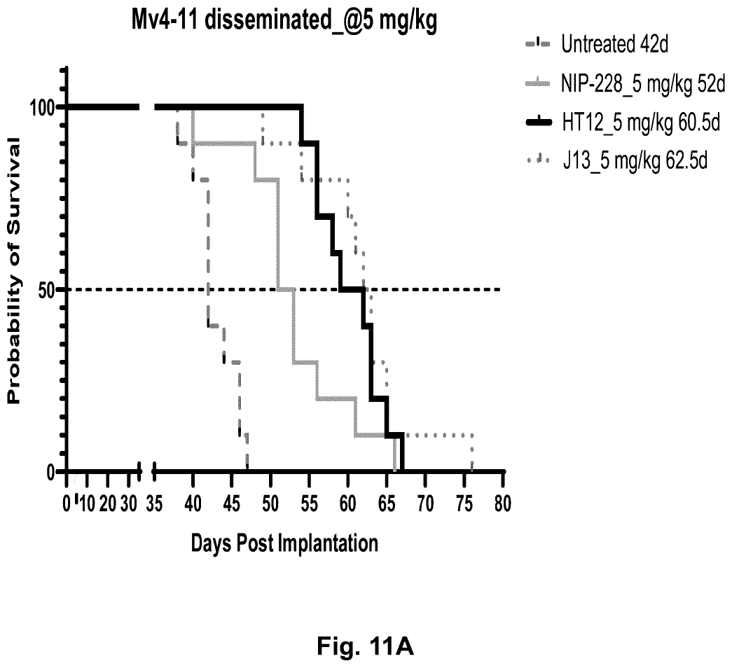

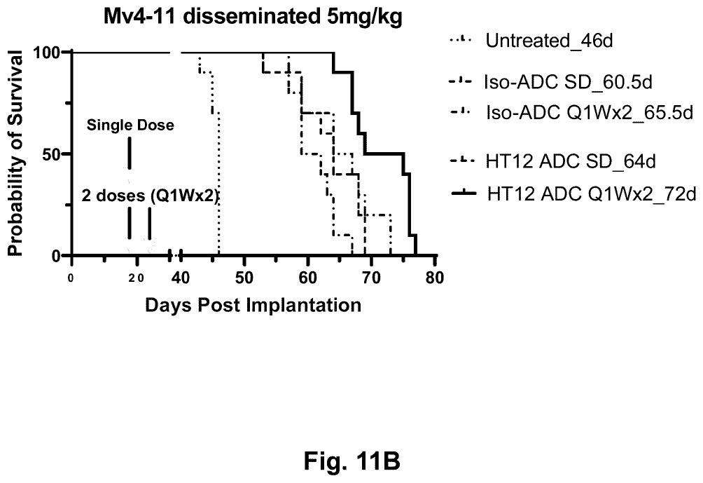

All leads induced survival benefit compared to untreated group in terms of medium survival. (FIG. 11A).

Mv4-11 cell implants were also tested with single and two doses of HT12-GL ADC. Mv4-11 cells were injected intravenously in the tail vein of female NSG mice as described at 5×10⁶ cells in 200 μL volume. Body weight was measured at day 5 after Mv4-11 cell implant. Mice were randomized based on body weight into treatment groups using the Matched Distribution method built into the Study Director software. Group designation, dose levels and number of mice per group are presented in the following table.

Test and control ADC were diluted from a stock solution with vehicle buffer (20 mM histidine, 240 mM sucrose, 0.02% Polysorbate 80, pH 6.0) and were administered as either a single intravenous (IV) injection or two IV injections (Q1W×2) according to body weight. The group destination and dose levels in the MV4-11 disseminated survival study are shown in Table 7.

TABLE 7

| | | | Route of | |
|---|---|---|---|---|
| | | Dose | administration, | Number |
| | | level | dosing regimen, | of |
| Group | Treatment | (mg/kg) | starting day | animals |
| 1 | Untreated | NA | NA | 10 |
| 4 | NIP-228-SG3932 | 5 mg/kg | IV, single doseat 19 | 10 |
| 5 | HT12-GL-SG3932 | 5 mg/kg | IV, single doseat 19 | 10 |
| 8 | NIP-228-SG3932 | 5 mg/kg | IV, Q1Wx2 at D19 | 10 |
| | | | and D26 | |
| 9 | HT12-GL-SG3932 | 5 mg/kg | IV, Q1Wx2 at D19 | 10 |
| | | | and D26 | |

Mice were monitored for signs of rough skin/fur and weak hind limbs. When the mice showed hind limb paralysis or incapability of reaching food/water, they were euthanized immediately and the survival days were recorded.

Weibull regression was fitted to the survival times, and hazard rate (better survival probability) and median survival were used to compare between groups.

Both single dosing and Q1W×2 dosing regimens of HT12-GL ADC induced modest survival benefit compared to isotype ADC-treated groups in terms of hazard rate (FIG. 11B).

### Example 15—In Vivo Patient Xenografts

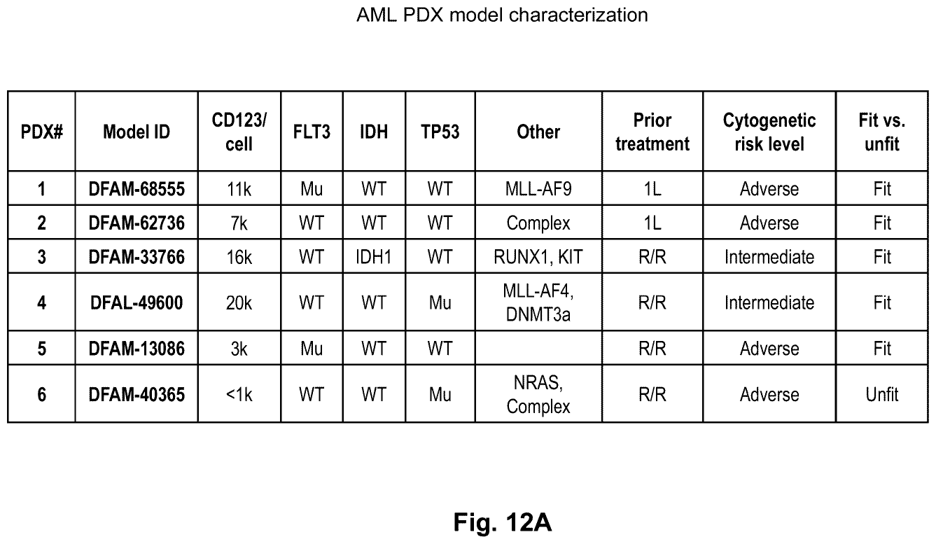

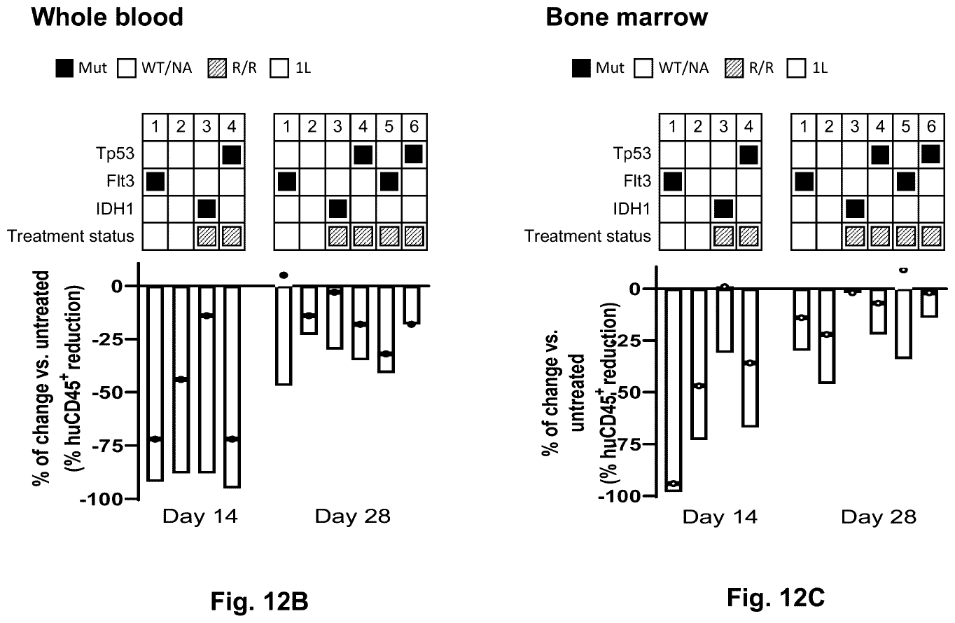

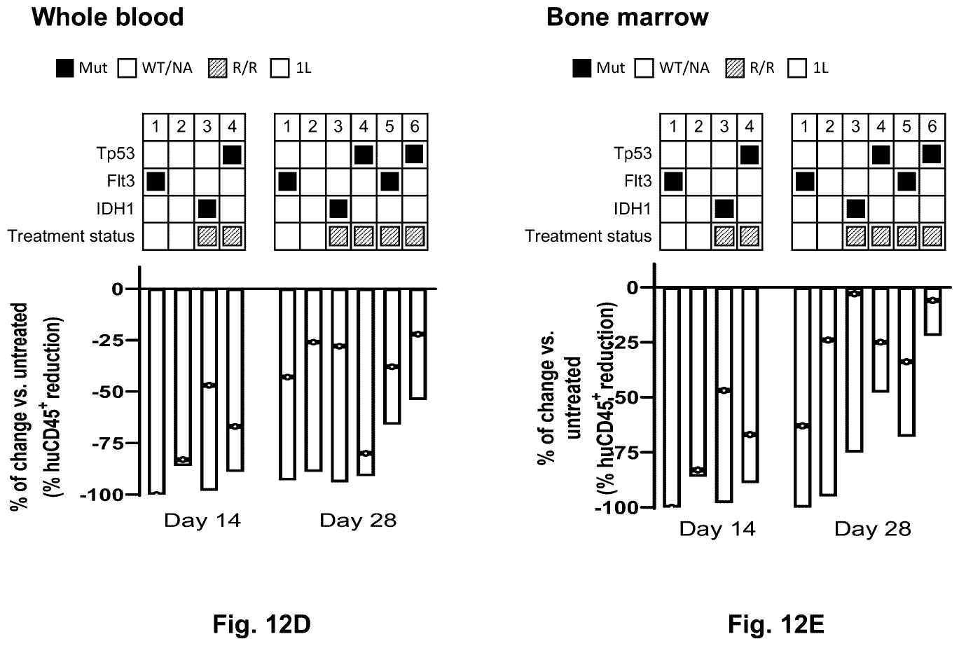

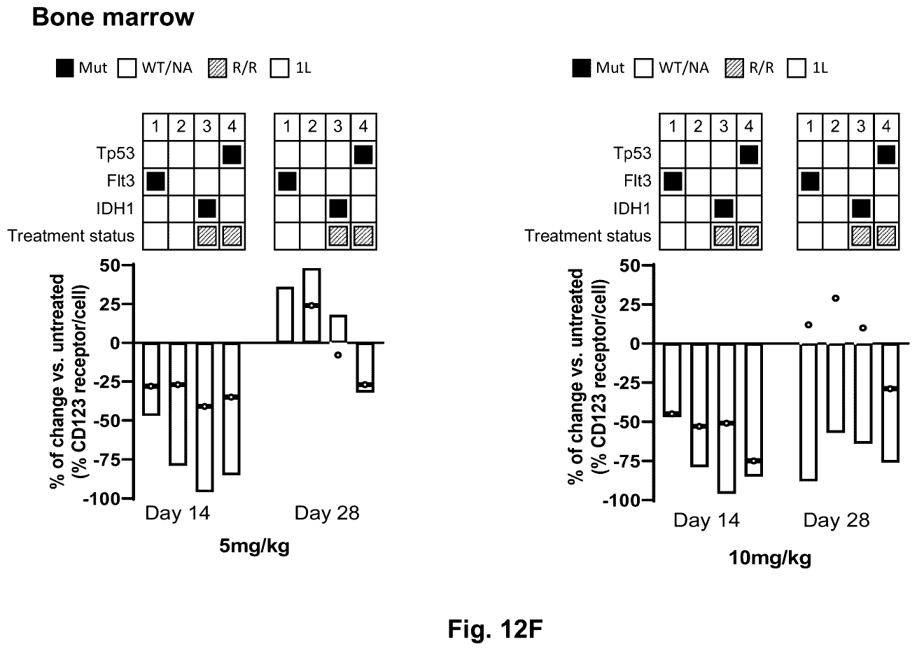

AML patient samples were used for Patient Derived Transplant Models (PDX). The AML PDX models characterization including the number of CD123 receptors per cell for each AML-PDX model (68555, 62736, 33766, 49600, 13086, and 40365) are shown in FIG. 12A.

For each AML-PDX model (68555, 49600, 33766, 62736, 13086, and 40365), cells were thawed and counted. Between 0.73 million cells and 1.4 million cells were intravenously implanted to NSG/NSG-SGM3 mice. Peripheral blood engraftment (% of huCD45) was sporadically checked by flow cytometric analysis from the whole cohort or subgroup of mice in order to determine the dosing stage. Either the peripheral blood engraftment or the body weight was used to randomize the mice using the Matched Distribution method built into the StudyLog software. Group designation, dose levels and number of mice per group are presented in Tables 7 and 8.

Test and control ADC were diluted from a stock solution with vehicle buffer (20 mM histidine, 240 mM sucrose, 0.02% Polysorbate 80, pH 6.0). All treated mice were given two intravenous (IV) injections according to body weight. The group designation and dose levels in AML-PDX disseminated end-point study models 68555, 62736, 49600, and 33766 are shown in Table 8.

TABLE 8

| | | Dose | | Route of | Number | End-point |
|---|---|---|---|---|---|---|
| | | level | | administration, | of | termination |
| Group | Treatment | (mg/kg) | | dosing regimen | animals | day |
| 1 | Untreated | NA | | NA | 5 | Day 14 post 1^{st} dose |
| 2 | NIP-228-SG3932 | 5 | mg/kg | IV, Q1Wx2 | 5 | Day 14 post 1^{st} dose |
| 3 | NIP-228-SG3932 | 10 | mg/kg | IV, Q1Wx2 | 5 | Day 14 post 1^{st} dose |
| 4 | HT12-GL-SG3932 | 5 | mg/kg | IV, Q1Wx2 | 5 | Day 14 post 1^{st} dose |
| 5 | HT12-GL-SG3932 | 10 | mg/kg | IV, Q1Wx2 | 5 | Day 14 post 1^{st} dose |
| 6 | Untreated | NA | | NA | 5 | Day 28 post 1^{st} dose |
| 7 | NIP-228-SG3932 | 5 | mg/kg | IV, Q1Wx2 | 5 | Day 28 post 1^{st} dose |
| 8 | NIP-228-SG3932 | 10 | mg/kg | IV, Q1Wx2 | 5 | Day 28 post 1^{st} dose |
| 9 | HT12-SG3932 | 5 | mg/kg | IV, Q1Wx2 | 5 | Day 28 post 1^{st} dose |
| 10 | HT12-GL-SG3932 | 10 | mg/kg | IV, Q1Wx2 | 5 | Day 28 post 1^{st} dose |

The day of 1st dose was defined as Day 0 in the study phase. Body weight was measured at least once per week. On Day 14, mice from group 1, 2, 3, 4, and 5 were euthanized. On Day 28, mice from group 6, 7, 8, 9, and 10 were euthanized.

The group designation and dose levels in AML-PDX disseminated end-point study models 13086 and 40365 are shown in Table 9.

TABLE 9

| | | Dose | | Route of | Number | End-point |
|---|---|---|---|---|---|---|
| | | level | | administration, | of | termination |
| Group | Treatment | (mg/kg) | | dosing regimen | animals | day |
| 1 | Untreated | NA | | NA | 5 | Day 28 post 1^{st} dose |
| 2 | NIP-228-SG3932 | 5 | mg/kg | IV, Q1Wx2 | 5 | Day 28 post 1^{st} dose |
| 3 | NIP-228-SG3932 | 6.5 | mg/kg | IV, Q1Wx2 | 5 | Day 28 post 1^{st} dose |
| 4 | NIP-228-SG3932 | 8 | mg/kg | IV, Q1Wx2 | 5 | Day 28 post 1^{st} dose |
| 5 | NIP-228-SG3932 | 10 | mg/kg | IV, Q1Wx2 | 5 | Day 28 post 1^{st} dose |
| 6 | HT12-GL-SG3932 | 5 | mg/kg | IV, Q1Wx2 | 5 | Day 28 post 1^{st} dose |
| 7 | HT12-GL-SG3932 | 6.5 | mg/kg | IV, Q1Wx2 | 5 | Day 28 post 1^{st} dose |
| 8 | HT12-GL-SG3932 | 8 | mg/kg | IV, Q1Wx2 | 5 | Day 28 post 1^{st} dose |
| 9 | HT12-GL-SG3932 | 10 | mg/kg | IV, Q1Wx2 | 5 | Day 28 post 1^{st} dose |

The day of 1st dose was defined as Day 0 in the study phase. Body weight was measured at least once per week. For models 13086 and 40365, on Day 28, mice from all groups were euthanized.

For all PDX models, the whole blood and bone marrow samples from individual mice were collected, processed, and stained for flow cytometric analysis. Percent of huCD45 was used to define anti-leukemic efficacy. CD123 receptor density was also assessed in bone marrow samples using flow cytometric analysis. Percentage change of each treated group was calculated by normalizing to time-point matched untreated group, except for model AML-PDX 68555, which was calculated by normalizing to day 14 untreated group. Waterfall was plotted for all AML-PDX models based on tissue matrix and dose level.

In whole blood at the dose level of 5 mg/kg, HT12-GL ADC induced significant anti-leukemic efficacy in 4/4 models at day 14 and 3/6 models at day 28 compared to untreated models (FIG. 12B). In bone marrow, HT12-GL ADC induced significant anti-leukemic efficacy at 5 mg/kg in 3/4 models at day 14 and 2/6 models at day 28 compared to untreated models (FIG. 12C).

In whole blood at the dose level of 10 mg/kg, HT12-GL ADC induced significant anti-leukemic efficacy in 4/4 models at day 14 and 6/6 models at day 28 compared to untreated models (FIG. 12D). In bone marrow, HT12-GL ADC induced significant anti-leukemic efficacy at 10 mg/kg in 4/4 models at day 14 and 4/6 models at day 28 compared to untreated models (FIG. 12E).

HT12-GL ADC also induced a robust decrease in the CD123 receptor density on the bone marrow samples at day 14 at 5 mg/kg and 10 mg/kg, suggesting a strong antigen-targeted mechanism (FIG. 12F). At day 28, CD123 receptor density was recovered at the lower dose of 5 mg/kg but not 10 mg/kg (FIG. 12F).

These results indicate a robust anti-leukemic efficacy of HT12-GL ADC across various AML-PDX models with different genetic mutations, treatment status and cytogenetic risk levels.

### Example 16—HT12-GL ADC Antibody and Payload Stability in CB17-SCID Mice

CB17-SCID mice were dosed with HT12-GL ADC at 5 mg/kg and plasma was analyzed by immunocapture of the antibody followed by either ELISA based detection of the human antibody or LCMS-enabled measurement of the heavy chain, light chain, and warhead components of the ADC.

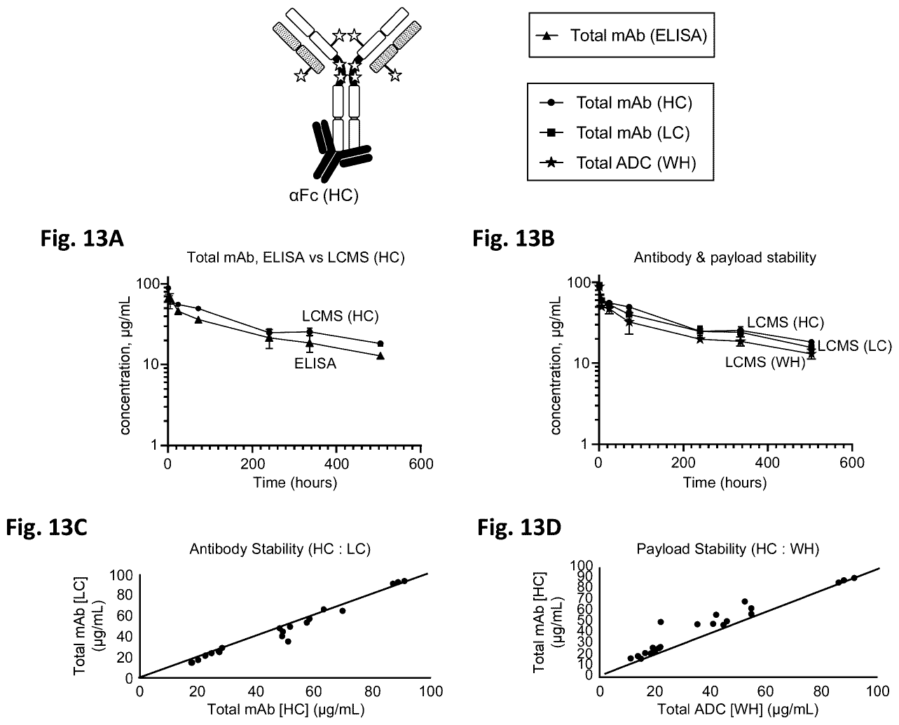

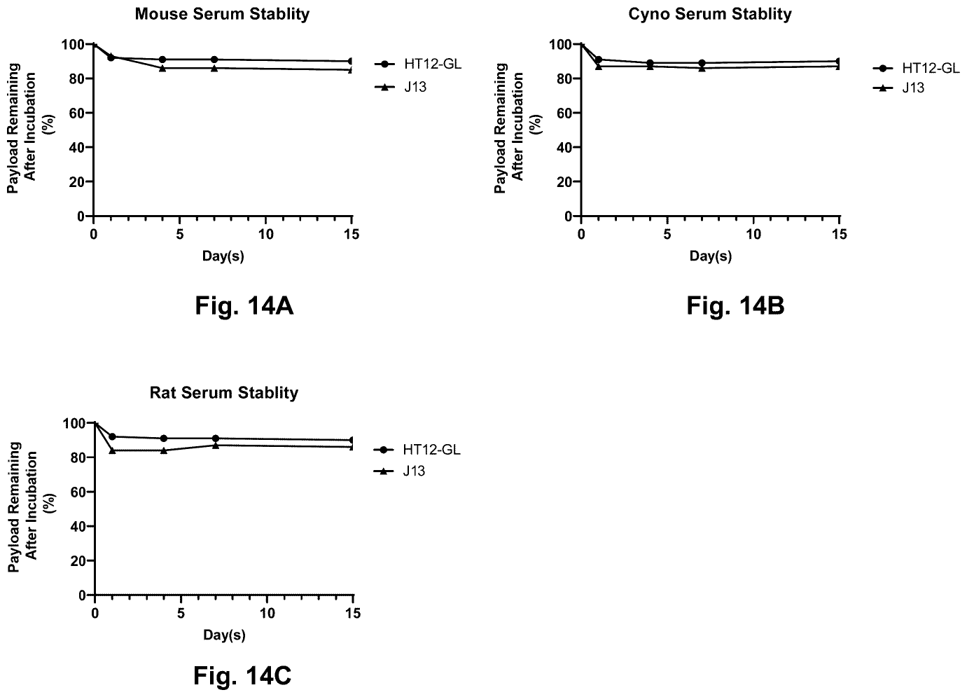

Exposure over time demonstrated antibody-like behavior of HT12-GL ADC, with PK parameters as follows: AUCinf of 821 (μg·days/mL), clearance of 6.1 mL/day/kg, and half-life of 14 days, based on ELISA results (FIG. 13A). Immunocapture of the HT12-GL ADC followed by trypsin digestion to produce a heavy chain peptide (HC) and a light chain peptide (LC) and papain digestion to release the warhead (WH) enabled targeted LC-MS/MS assessment of antibody and payload stability (FIG. 13B). Comparison of exposure between surrogate analytes suggested that the HT12-GL ADC was stable in vivo (FIGS. 13C and 13D). HT12-GL ADC was also stable in mouse serum (FIG. 14A), cynomolgus serum (FIG. 14B), and rat serum (FIG. 14C).

### Example 17—Synthesis of Topoisomerase I Inhibitors

### General Information

Flash chromatography was performed using a Biotage® Isolera™ and fractions checked for purity using thin-layer chromatography (TLC). TLC was performed using Merck Kieselgel 60 F254 silica gel, with fluorescent indicator on aluminium plates. Visualisation of TLC was achieved with UV light.

Extraction and chromatography solvents were bought and used without further purification from VWR U.K.

All fine chemicals were purchased from Sigma-Aldrich unless otherwise stated. Pegylated reagents were obtained from Quanta biodesign US via Stratech UK.

### LC/MS Conditions

### Method A

Positive mode electrospray mass spectrometry was performed using a Waters Aquity H-class SQD2. Mobile phases used were solvent A (water with 0.1% formic acid) and solvent B (acetonitrile with 0.1% formic acid). Initial composition 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes. Flow rate was 0.8 mL/minute. Detection was at 254 nm. Columns: Waters Acquity UPLC® BEH Shield RP18 1.7 μm 2.1×50 mm at 50° C. fitted with Waters Acquity UPLC® BEH Shield RP18 VanGuard Pre-column, 130A, 1.7 μm, 2.1 mm×5 mm.

### Method B

The HPLC (Waters Alliance 2695) was run using a mobile phase of water (A) (formic acid 0.1%) and acetonitrile (B) (formic acid 0.1%). Initial composition 5% B held over 25 seconds, then increased from 5% B to 100% B over a 1 minute 35 seconds' period. The composition was held for 50 seconds at 100% B, then returned to 5% B in 5 seconds and held there for 5 seconds. The total duration of the gradient run was 3.0 minutes. Flow rate was 0.8 mL/minute. Wavelength detection range: 190 to 800 nm. Columns: Waters Acquity UPLC® BEH Shield RP18 1.7 μm 2.1×50 mm at 50° C. fitted with Waters Acquity UPLC® BEH Shield RP18 VanGuard Pre-column, 130A, 1.7 μm, 2.1 mm×5 mm.

### Method C

The HPLC (Waters Alliance 2695) was run using a mobile phase of water (A) (formic acid 0.1%) and acetonitrile (B) (formic acid 0.1%).

Initial composition 5% B held over 1 min, then increase from 5% B to 100% B over a 9 min period. The composition was held for 2 min at 100% B, then returned to 5% B in 0.10 minutes and hold there for 3 min. Total gradient run time equals 15 min. Flow rate 0.6 mL/min. Wavelength detection range: 190 to 800 nm. Oven temperature: 50° C. Column: ACE Excel 2 C18-AR, 2μ, 3.0×100 mm.

HPLC conditions

Reverse-phase ultra-fast high-performance liquid chromatography (UFLC) was carried out on a Shimadzu Prominence™ machine using a Phenomenex™ Gemini NX 5μ C18 column (at 50° C.) dimensions: 150×21.2 mm. Eluents used were solvent A (H₂O with 0.1% formic acid) and solvent B (CH₃CN with 0.1% formic acid). All UFLC experiments were performed with gradient conditions: Initial composition 13% B increased to 30% B over a 3 minutes period, then increased to 45% B over 8 minutes and again to 100% over 6 minutes before retunning to 13% over 2 min and hold for 1 min. The total duration of the gradient run was 20.0 minutes. Flow rate was 20.0 mL/minute and detection was at 254 and 223 nm.

### NMR Method

Proton NMR chemical shift values were measured on the delta scale at 400 MHz using a Bruker AV400. The following abbreviations have been used: s, singlet; d, doublet; t, triplet; q, quartet; quin, quintet; m, multiplet; br, broad. Coupling constants are reported in Hz.

### Synthesis of Key Intermediates:

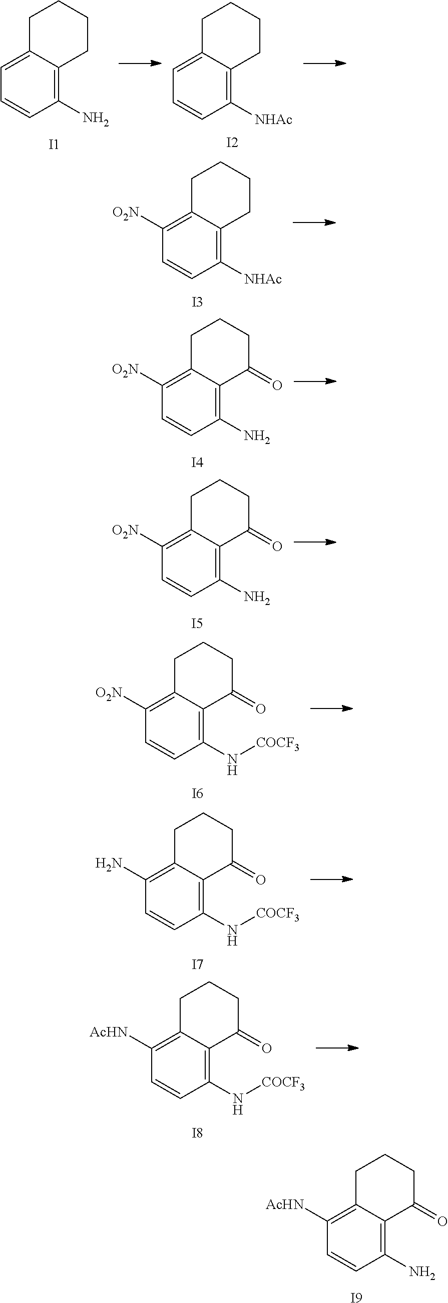

### a) N-(5,6,7,8-tetrahydronaphthalen-1-yl)acetamide (I2)

5,6,7,8-tetrahydronaphthalen-1-amine I1 (8.54 g, 58.0 mmol) was dissolved in dichloromethane (80 mL). Triethylamine (18 mL, 129 mmol) was added and the mixture cooled to 0° C. Dropwise, acetic anhydride (11.5 mL, 122 mmol) was added, upon completion of the addition, the reaction mixture was warmed to rt and stirred for 45 min, whereupon LCMS indicated the reaction was complete. The mixture was diluted with CH₂Cl₂, washed with H₂O, sat. NaHCO₃, 10% citric acid, the organic phase dried over MgSO₄ and concentrated in vacuo. The off-white solid was triturated with 1:3 Et₂O/isohexane to afford 12 (10.8 g, 57.1 mmol, 98% Yield) as a white solid which was used without further purification. LC/MS (method A): retention time 1.44 mins (ES+) m/z 190 [M+H]⁺

### b) N-(4-nitro-5,6,7,8-tetrahydronaphthalen-1-yl)acetamide (I3)

N-(5,6,7,8-tetrahydronaphthalen-1-yl)acetamide I2 (1.00 g, 5.2840 mmol) was added portion-wise to sulfuric acid (15 mL, 281 mmol) at −5° C. Sodium nitrate (450 mg, 5.2945 mmol) was added portion-wise to the reaction mixture and stirred for 30 min at −5° C. whereupon LCMS indicated no further reaction progress. The reaction mixture was poured onto ice with external cooling, the aqueous mixture extracted with CH₂Cl₂, the organic phase dried over MgSO₄ and purified by Isolera (10-80% EtOAc in isohexane) to afford a mixture of N-(4-nitro-5,6,7,8-tetrahydronaphthalen-1-yl)acetamide 13 and N-(2-nitro-5,6,7,8-tetrahydronaphthalen-1-yl)acetamide (956 mg, 4.0811 mmol, 77% Yield) as a white/yellow solid. LC/MS (method A): retention time 1.53 mins (ES+) m/z 235 [M+H]⁺.

### c) N-(4-nitro-8-oxo-5,6,7,8-tetrahydronaphthalen-1-yl)acetamide (I4)

N-(4-nitro-5,6,7,8-tetrahydronaphthalen-1-yl)acetamide I3 (1.01 g, 4.31 mmol) was dissolved in acetone (30 mL). Magnesium sulfate in water (3.9 mL, 5.9 mmol, 1.5 mol/L) was added and the mixture was cooled to 0° C. Potassium permanganate (2.07 g, 13.0 mmol) was added portionwise to the reaction mixture and the mixture warmed to rt and stirred for 50 min, whereupon TLC indicated the reaction was complete. The reaction mixture was filtered through Celite, the solids washed with CHCl₃ and the resulting organic mixture washed with H₂O, brine, dried over MgSO₄ and purified by isolera (20-50% EtOAc in isohexane) to afford a mixture of N-(4-nitro-8-oxo-5,6,7,8-tetrahydronaphthalen-1-yl)acetamide 14 and N-(2-nitro-8-oxo-5,6,7,8-tetrahydronaphthalen-1-yl)acetamide (709 mg, 2.86 mmol, 66%) as a white/yellow solid. LC/MS (method A): retention time 1.44 mins (ES+) m/z 190 [M+H]⁺

### d) 8-amino-5-nitro-3,4-dihydronaphthalen-1(2H)-one (I5)

A mixture of N-(4-nitro-8-oxo-5,6,7,8-tetrahydronaphthalen-1-yl)acetamide 14 and N-(2-nitro-8-oxo-5,6,7,8-tetrahydronaphthalen-1-yl)acetamide (709 mg, 2.8561 mmol) and 6N hydrochloric acid (7 mL) were stirred at 80° C. for 2.5 h, whereupon LCMS indicated the reaction was complete. The reaction mixture was cooled in an ice bath and 6N NaOH solution was added until the pH was basic. The aqueous mixture was extracted with CH₂Cl₂, the organic phase dried over MgSO₄ and concentrated in vacuo. Isolera (0-50% EtOAc in isohexane) afforded 8-amino-5-nitro-3,4-dihydronaphthalen-1(2H)-one 15 (320 mg, 1.552 mmol, 54% Yield) as a yellow/orange solid. LC/MS (method A): retention time 1.54 mins (ES+) m/z 207 [M+H]⁺

### e) 2,2,2-trifluoro-N-(4-nitro-8-oxo-5,6,7,8-tetrahydronaphthalen-1-yl)acetamide (I6)

8-amino-5-nitro-3,4-dihydronaphthalen-1(2H)-one 15 (430 mg, 2.0854 mmol) was dissolved in dichloromethane (20 mL). Pyridine (340 μL, 4.20 mmol) was added and the mixture cooled to 0° C. Trifluoroacetic anhydride (590 μL, 4.197 mmol) was added and stirred for 30 min, whereupon LCMS indicated the reaction was complete. The mixture was diluted with CH₂Cl₂, washed with H₂O, the organic phase dried over MgSO₄ and concentrated in vacuo to afford 2,2,2-trifluoro-N-(4-nitro-8-oxo-5,6,7,8-tetrahydronaphthalen-1-yl)acetamide 16 (630 mg, 2.0846 mmol, >99% Yield) as a yellow solid, which was used without further purification. LC/MS (method A): retention time 1.86 min (ES+) m/z 301X [M−H]⁻

### f) N-(4-amino-8-oxo-5,6,7,8-tetrahydronaphthalen-1-yl)-2,2,2-trifluoroacetamide (I7)

Zinc (2.73 g, 41.7 mmol) was suspended in methanol (80 mL), formic acid (4 mL) and water (4 mL) and the mixture cooled to 0° C. 2,2,2-trifluoro-N-(4-nitro-8-oxo-5,6,7,8-tetrahydronaphthalen-1-yl)acetamide 16 (568 mg, 2.0865 mmol) was added portion-wise and the mixture stirred at 0° C. for 30 min, whereupon LCMS indicated the reaction was complete. The reaction mixture was filtered, the filtrate diluted with EtOAc and washed with sat NaHCO₃. The organic phase was dried over MgSO₄ and concentrated in vacuo to afford N-(4-amino-8-oxo-5,6,7,8-tetrahydronaphthalen-1-yl)-2,2,2-trifluoroacetamide 17 (568 mg, 2.0865 mmol, >99% Yield) as a yellow solid, which was used without further purification. LC/MS (method A): retention time 1.65 min (ES+) m/z 273 [M+H]⁺

### g) N-(4-acetamido-8-oxo-5,6,7,8-tetrahydronaphthalen-1-yl)-2,2,2-trifluoroacetamide (I8)

N-(8-amino-4-oxo-tetralin-5-yl)-2,2,2-trifluoro-acetamide 17 (568 mg, 2.0865 mmol) was dissolved in dichloromethane (20 mL). Triethylamine (580 μL, 4.16 mmol) then acetyl chloride (297 L, 4.173 mmol) were added and the mixture stirred for 30 min, whereupon LCMS indicated the reaction was complete. The reaction mixture was diluted with CH₂Cl₂, washed with H₂O, the organic phase dried over MgSO₄ and concentrated in vacuo to afford N-(8-acetamido-4-oxo-tetralin-5-yl)-2,2,2-trifluoro-acetamide 18 (655 mg, 2.084 mmol, >99% yield) as a yellow solid, which was used without further purification. LC/MS (method A): retention time 1.55 min (ES+) m/z 315 [M+H]⁺.

### h) N-(4-amino-5-oxo-5,6,7,8-tetrahydronaphthalen-1-yl)acetamide (I9)

N-(8-acetamido-4-oxo-tetralin-5-yl)-2,2,2-trifluoro-acetamide 18 (2.77 g, 8.81 mmol) was dissolved in methanol (240 mL) and water (17 mL). Potassium carbonate (4.88 g, 35.3 mmol) was added and the mixture stirred for 1.5 h at 50° C., whereupon LCMS indicated the reaction was complete. The reaction mixture was cooled, concentrated in vacuo, dissolved in 10% MeOH in CH₂Cl₂ and washed with H₂O. The organic phase was dried over MgSO₄ and purified by isolera chromatography (2-15% MeOH in CH₂Cl₂) to afford N-(8-amino-1-oxo-tetralin-5-yl)acetamide 19 (1.20 g, 5.50 mmol, 62.3% Yield) as a yellow solid. LC/MS (method A): retention time 0.98 min (ES+) m/z 219 [M+H]⁺.

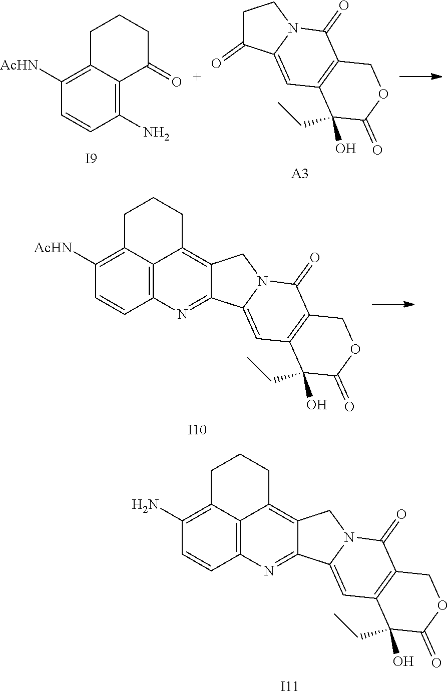

### i) (S)—N-(9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)acetamide (I10)

N-(8-amino-1-oxo-tetralin-5-yl)acetamide 19 (641 mg, 2.94 mmol, 1.0 eq.), (S)-4-ethyl-4-hydroxy-7,8-dihydro-1H-pyrano[3,4-f]indolizine-3,6,10(4H)-trione A3 (840 mg, 3.19 mmol, 1.1 eq.) and PPTS (740 mg, 2.95 mmol, 1.0 eq.) were dissolved in toluene (60 mL) and stirred at reflux for 3 h, whereupon LCMS indicated 19 had been consumed. The reaction mixture was cooled and concentrated in vacuo. The resulting solids were triturated with acetonitrile, then acetone to afford 110 as a brown solid with minor TsOH contamination (1.26 g, 96%). LC/MS (method A): retention time 1.32 mins (ES+) m/z 447 [M+H]⁺.

### j) (S)-4-amino-9-ethyl-9-hydroxy-1,2,3,9,12,15-hexahydro-10H,13H-benzo[de]pyrano[3′,4′:6,7]indolizino[1,2-b]quinoline-10,13-dione (I11)

(S)—N-(9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)acetamide (I10) (1.26 g, 2.83 mmol, 1.0 eq.) was dissolved in hydrochloric acid (6 mol/L) in H₂O (12 mL) and the mixture stirred for 5 h at 80° C., whereupon LCMS indicated 110 had been consumed. The reaction mixture was diluted with H₂O and concentrated in vacuo to afford (S)-4-amino-9-ethyl-9-hydroxy-1,2,3,9,12,15-hexahydro-10H,13H-benzo[de]pyrano[3′,4′:6,7]indolizino[1,2-b]quinoline-10,13-dione I11 (1.51 g, 2.85 mmol, 90 mass %, 101% Yield) as a red crystalline solid. LC/MS (method A): retention time 1.36 mins (ES+) m/z 405 [M+H]⁺.

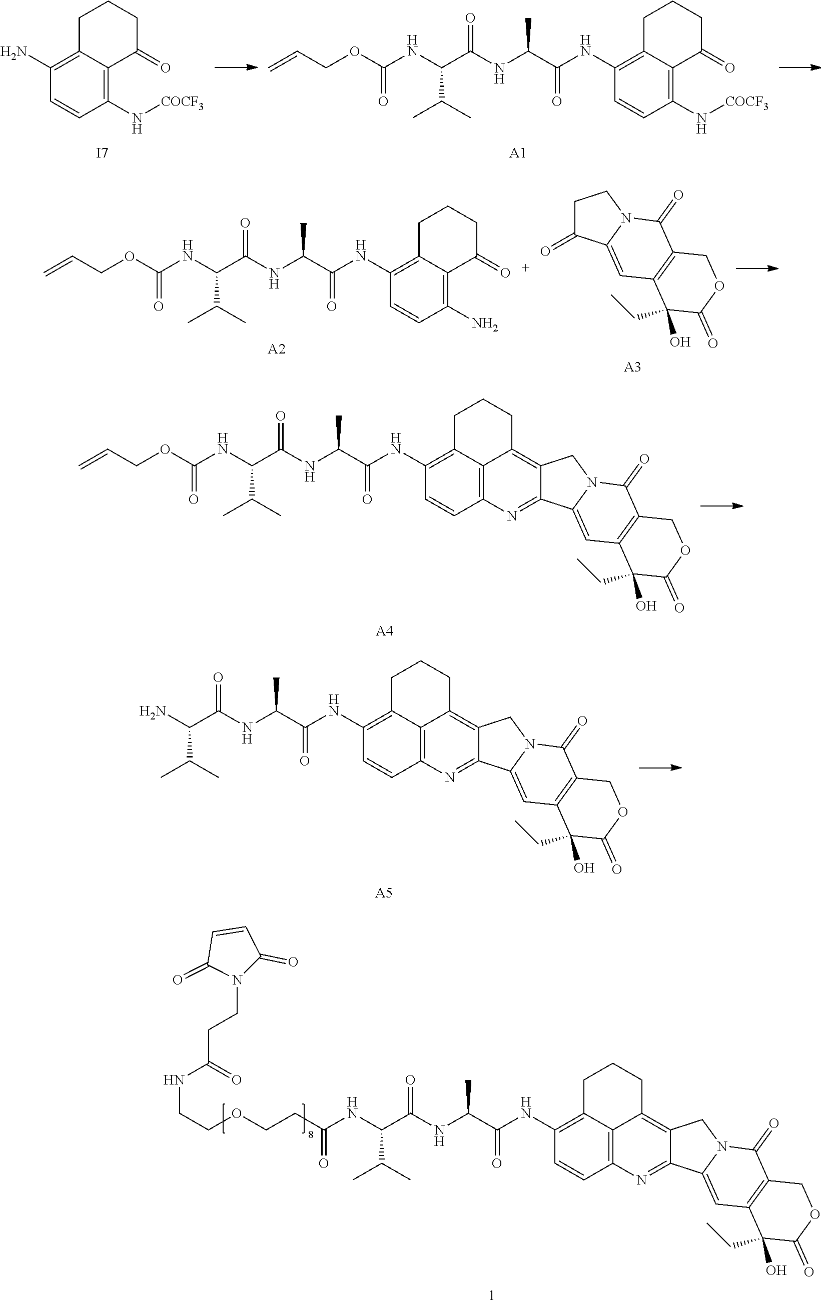

### a) Allyl ((S)-3-methyl-1-oxo-1-(((S)-1-oxo-1-((5-oxo-4-(2,2,2-trifluoroacetamido)-5,6,7,8-tetrahydronaphthalen-1-yl)amino)propan-2-yl)amino)butan-2-yl)carbamate (A1)

DCC (6.54 g, 31.7 mMol) and HOPO (3.36 g, 30.2 mMol) were added to a solution of alloc-Val-Ala-OH (9.09 g, 31.7 mmol) and 17 (7.85 g, 28.8 mMol) in CH₂Cl₂ (300 mL) at 25° C. The resulting mixture was left to stir overnight. The white solid that formed during the reaction was filtered out and washed with cold CH₂Cl₂. The filtrate was washed with water (150 mL) and brine (150 mL). The organic layer was dried over MgSO₄, filtered and evaporated. The crude product was purified by silica gel chromatography (Hex/EtOAc, 60:40). Product A1 isolated was contaminated with co-eluting DCU (21.1 g, 140% yield). LC/MS (Method B): ES⁺=1.81 min, m/z 527.6 [M+H]⁺.

### b) Allyl ((S)-1-(((S)-1-((4-amino-5-oxo-5,6,7,8-tetrahydronaphthalen-1-yl)amino)-1-oxopropan-2-yl)amino)-3-methyl-1-oxobutan-2-yl)carbamate (A2)

Protected aniline A1 (18 g, 34.19 mMol) was solubilised in a mixture of MeOH and H₂O 10:1 (165 mL) and K₂CO₃ was added (10 g, 72.36 mMol). The mixture was stirred at 50° C. until complete. The mixture was vacced down to almost dryness and the residue was taken up with CH₂Cl₂ and washed with H₂O and brine, before being dried over MgSO₄, filtered and evaporated. The crude product was purified by silica gel chromatography (CHCl₃/MeOH, 100% to 7:3). The isolated product A2 was contaminated with a co-eluting impurity (10.71 g, 73% yield). LC/MS (Method B): ES⁺=1.46 min, m/z 431.7 [M+H]⁺.

### c) Allyl ((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methylbutan-2-yl)carbamate (A4)

Aniline A2 (450 mg, 1.045 mMol), lactone A3 (280 mg, 1.064 mMol) and pyridinium p-toluenesulfonate (273 mg. 1,086 mMol) were solubilised in toluene (20 mL) and the mixture was heated to 130° C. (high reflux). Every now and then a few drops of MeOH is added to help solubilise the mixture. After 7 h the crude reaction was vacced down to dryness. The crude product was purified by silica gel chromatography (CHCl₃/MeOH, 100% to 95:5) to give product A4 (360 mg, 52.3% yield). LC/MS (Method B): ES⁺=1.51 min, m/z 658.8 [M+H]⁺.

### d) Allyl (S)-2-amino-N—((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)-3-methylbutanamide (A5)

Excess piperidine was added (642 μL) to a solution of A4 (543 mg, 0.82 mMol) and PdP(Ph₃)₄ (89 mg, 0.08 mMol) in CH₂Cl₂ (15 mL). The mixture was allowed to stir at room temperature for 20 min, at which point the reaction had gone to completion (as monitored by LC/MS). The reaction mixture was diluted with CH₂Cl₂ (25 mL) and the organic phase was washed with H₂O (25 mL) and brine (25 mL). The organic phase was dried over MgSO₄, filtered and excess solvent removed by rotary evaporation under reduced pressure to afford crude product A5 which was used as such in the next step. LC/MS (Method B): ES⁺=1.15 min, m/z 574.6 [M+H]⁺.

### e) 1-(3-(2,5-dioxo-2,5-dihydro-1H-pyrrol-1-yl)propanamido)-N—((S)-1-(((S)-1-(((S)-9-ethyl-9-hydroxy-10,13-dioxo-2,3,9,10,13,15-hexahydro-1H,12H-benzo[de]pyrano[3′,4′:6,7]indolizino[1,2-b]quinolin-4-yl)amino)-1-oxopropan-2-yl)amino)-3-methyl-1-oxobutan-2-yl)-3,6,9,12,15,18,21,24-octaoxaheptacosan-27-amide (1)

Pyridine (83 μL, 1.03 mMol) and Mal-dPEG₈-OTFP (767 mg, 1.03 mMol) were added to a solution of crude A5 (assumed 1.03 mMol) in dry CH₂Cl₂ (50 mL) under an argon atmosphere. The reaction was stirred overnight and as the reaction was not complete 0.5 eq. of Mal-dPEG₈-OTFP was added to try to push the reaction. The reaction was diluted with CH₂Cl₂(25 mL) and the organic phase was washed with H₂O (2×50 mL) and brine before being dried over MgSO₄, filtered and excess solvent removed by rotary evaporation under reduced pressure by rotary evaporation under reduced pressure. The crude was purified by reverse phase HPLC (gradient of H₂O/CH₃CN+0.05% FA) and freeze dried to give 1 (1.189 g, 31% yield over 2 steps). LC/MS (Method B): ES⁺=1.43 min, m/z 1149.3 [M+H]⁺. LC/MS (Method C): ES⁺=5.37 min, m/z 1149.4 [M+H]⁺.

### Example 18—HT12-GL-SG3932 Stability in Human Serum

ADCs bearing drug linked to cysteines via a thiosuccinimide are known to exhibit some drug loss in physiological milieu due to the retro-Michael reaction. This process regenerates the cysteine used for conjugation and the maleimide-bearing drug, thus reducing the DAR of the ADC over time. This deconjugation process is a known property of ADCs containing drug linked to antibodies through thiosuccinimides.

Serum stability assays were conducted by incubation of HT12-GL-SG3932 in human serum for 15 days at 37° C. to approximate physiological conditions. All antibody species, including conjugated and unconjugated ADC, were recovered following serum incubation by immunocapture using anti-human Fc sepharose resin. Captured species were eluted from resin and analyzed by rLC/MS to determine the relative amounts of conjugated and unconjugated species from peak heights in the mass spectra as previously described (Wiggens et al, 2015, Vallier-Douglas et al, 2012, Xu et al, 2011). Amount of conjugated drug at given timepoints was converted to a total DAR value by combining the amount of drug on antibody light-chain and heavy-chain species.

HT12-GL-SG3932 (187 μL of 3.2 mg/mL stock) was added to 3 mL serum and the mixture was sterilized by passing through a 0.2 μm filter. An aliquot (600 μL) of this solution was removed and frozen as the time=0 sample. Remaining ADC-serum solution was sealed in a vial and incubated at 37° C. with gentle mixing. Aliquots (600 μL) were subsequently removed on days 1,4,7, and 15 and frozen at −80° C. until MS analysis. The ADC was recovered from serum by immunocapture using 5F12G3, an anti-HT12 anti-paratope antibody, coupled to Dynabeads™ Myone™ Streptavidin T1 beads.

The 5F12G3 anti-paratope antibody was generated by immunizing Blab C mice with the Fab region of the HT12 antibody and generating a hybridoma clones from the mice B cells. These clones were screened for specific binding to HT12-GL and HT12-GL-SG3932 and clone 5F12G3 was identified to specifically bind with high affinity to HT12-GL and HT12-GL-SG3932. 5F12G3 was biotinylated, following manufacture recommendations, (ThermoFisher, CA) and coupled to Dynabeads™ MyOne™ Streptavidin T1 beads (ThermoFisher, CA). The 5F12G3 magnetic Dynabeads were rinsed three times with PBS pH 7.2, once with 0.1 M glycine pH 3.5, and then twice more with PBS pH 7.2 prior to use. ADC-serum samples were then combined with 5F12G3 magnetic Dynabeads (100 μL of ADC-serum mixture, 50 μL slurry) and mixed for 60 minutes at room temperature. The 5F12G3 magnetic Dynabeads were sedimented using a magnetic stand and then washed three times with PBS pH 7.2. Washed Dynabeads were resuspended in 100 μL 0.1 M glycine pH 3.5 and further incubated for 5 minutes at room temperature. The Dynabeads were sedimented using a magnetic stand and elutant removed and added to 10 μL of 1 M Tris-HCl (ThermoFisher, Waltham, MA USA). Recovered human antibody solutions (45 μL) were then reduced with DTT (Pierce, Rockford, IL USA) (5 μL) and analyzed by LC/MS.

rLC/MS analysis was conducted on an Agilent 1290 series HPLC coupled to an Agilent 6520 Accurate-Mass TOF LC/MS (Santa Clara, CA USA) with an electrospray ionization source. Approximately 2 μg (45 μL of volume) of reduced serum stability eluants were loaded onto a Poroshell 300SB-C3 column (2.1×75 mm, 255) (Agilent, Santa Clara, CA USA) and eluted at a flow rate of 0.4 mL/minute using a step gradient of solvent A to 60% solvent B after 6 minutes (solvent A: 0.1% formic acid in water; solvent B: 0.1% formic acid in acetonitrile) (J. T. Baker, Radnor, PA USA). The total mass spectrum was obtained by integrating the entire peak from the chromatogram, which contained both light-chain and heavy-chain antibody species. The total mass spectrum was then deconvoluted using Agilent MassHunter data acquisition and chromatogram processing software (Agilent, Santa Clara, CA USA).

Peak height intensities of conjugated and unconjugated species in deconvoluted mass spectra were used to calculate the DAR for each sample. Hydrolyzed thiosuccinimide linkages (+18 amu) were included as “conjugated species” in the analysis. The following equations were used:

$$Calculation of light chain DAR \qquad Equation 1$$

$${DAR}_{LC} = [ ( LC + 1 ) + ( LC + 1 hydrolyzed ) ] /$$

$$[ LC + ( LC + 1 ) + ( LC + 1 hydrolyzed ) ]$$

$$LC = peak height of light chain$$

$$LC + 1 = peak height of light chain + AZ 14170133$$

$$LC + 1 hydrolyzed = peak height of light chain +$$

$$AZ 14170133 + hydrolyzed thiosuccinimide species$$

$$Calculation of heavy chain DAR \qquad Equation 2$$

$${DAR}_{HC} = [ ( HC + 1 ) + ( HC + 1 hydrolyzed ) + 2 \times ( ( HC + 2 ) + 2 \times$$

$$( HC + 2 hydrolyzed ) + 3 \times ( HC + 3 ) + 3 \times ( ( HC + 3 hydrolyzed ) ] /$$

$$[ HC + ( HC + 1 ) + ( HC + 1 hydrolyzed ) + 2 \times ( HC + 2 ) + 2 \times$$

$$( HC + 2 hydrolyzed ) + 3 \times ( HC + 3 ) + 3 \times ( HC + 3 hydrolyzed ) ]$$

$$HC = peak height of heavy chain$$

$$HC + 1 = peak height of heavy chain + AZ 14170133$$

$$HC + 1 hydrolyzed = peak height of heavy chain +$$

$$AZ 14170133 + hydrolyzed thiosuccinimide species$$

$$HC + 2 = peak height of heavy chain + 2 AZ 14170133$$

$$HC + 2 hydrolyzed = peak height of heavy chain +$$

$$2 AZ 14170133 + hydrolyzed thiosuccinimide species$$

$$HC + 3 = peak height of heavy chain + 3 AZ 14170133$$

$$HC + 3 hydrolyzed = peak height of heavy chain +$$

$$3 AZ 14170133 + hydrolyzed thiosuccinimide species$$

$$Calculation of Total DAR \\ {DAR}_{total} = 2 \times ( {DAR}_{LC} + {DAR}_{HC} ) \qquad Equation 3$$

Drug loss occurred for HT12-GL-SG3932 over time following incubation in human at 37° C. compared to the time zero sample, evidenced by appearance of unconjugated antibody light chain and heavy chain peaks in reduced MS analysis. Drug loss through linker cleavage was not observed, which would result from enzyme activity. However, unmodified antibody peaks (i.e. regeneration of unmodified cysteine residues via loss of the entire AZ14170133 drug-linker) increased over time. These two observations confirm that drug loss in human serum occurred through a retroMichael reaction.

Less than 18.5% drug loss occurs from HT12-GL-SG3932 after 15 days incubation in Human serum. The mechanism of drug release is deconjugation through the retro-Michael reaction and not linker cleavage. This observation is consistent with other ADCs prepared by maleimide conjugation to cysteine amino acids involved with interchain disulfides.

### Example 19—HT12-GL-SG3932 Combination Therapy with Standard of Care (HMA and Venetoclax) or with Cytarabine in AML Cell Lines

The effects of HT12-GL-SG3932 ADC (AZD9829) combination therapy with the standard of care (SoC) combination of DNA hypomethylating agent (HMA) and BCL2 inhibitor Venetoclax (VEN), and HT12-GL-SG3932 ADC combination therapy with Cytarabine (Cytosine arabinoside) was investigated in AML cell lines in vitro.

AML cell lines were cultured in vitro for 6 days with one of the following treatments groups: (1) HT12-GL-SG3932, (2) VEN, (3) HMA (Decitabine), (4) Cytarabine, (5) a double combination of HT12-GL-SG3932 and VEN, (6) a double combination of HT12-GL-SG3932 and HMA, (7) a double combination of HT12-GL- and Cytarabine, or (8) a triple combination of HT12-GL-SG3932 with VEN and HMA. Seven different CD123-expressing AML cell lines with a variety of molecular alterations (i.e., MOLM-13, EOL-1, KASUMI-1, KG-1, THP-1, NOMO-1, OCI-AML3, and OCI-AML5 cells) were tested and untreated cells of each cell line were used as controls. 20 uL media (RPMI+10% FBS, IMDM+10% FBS, or αMEM+20% FBS) was prefilled into each well of 384-well plates using a MultiDrop Combi dispenser. Test agents were then added to each plate using the Plate Reformat program on an ECH0655 or ECH0550 acoustic dispenser. HT12-GL-SG3932 (in aqueous buffer, 40-250 nL/well), venetoclax (in DMSO, 40 nL/well), and/or decitabine (in DMSO, 40 nL/well), Cytarabine (in DMSO, 40 nL/well) were added in triplicate according to a 12*6 dose matrix. Drug concentrations were chosen for each cell line individually to focus on doses which display moderate potency to allow combination benefit to be assessed. Cell suspension (20 uL/well) was then added into treated plates using a multichannel pipette. Plates were then incubated for 6 days. At the end of the assay, CellTiter-Glo2.0 (Promega G9242, 40 uL/well) was added to each well of plates using multichannel pipette or MultiDrop Combi dispenser. Plates were incubated at room temperature in the dark for 10⁻¹⁵ minutes, then luminescence was read using an Envision plate reader to assess cell viability. Data was analyzed in Rstudio and combination benefit was assessed using the Synergyfinder package in R. Bliss scores≥5 concurrent with <50% cell viability was interpreted to represent meaningful synergy.

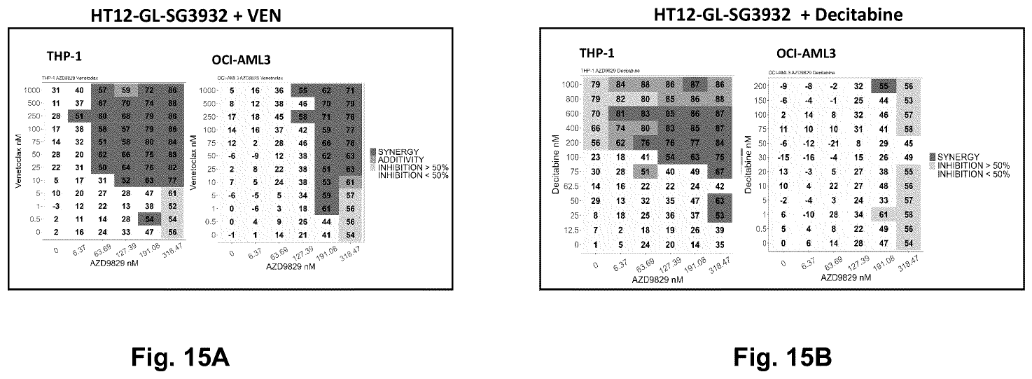

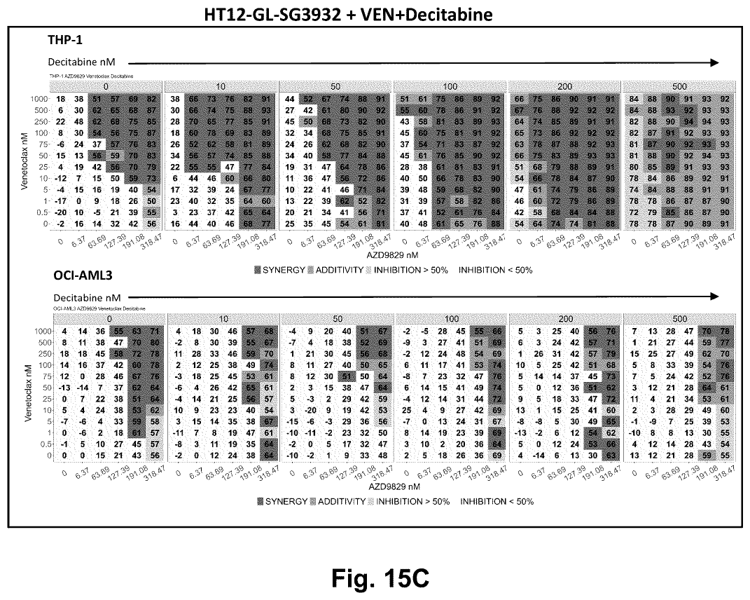

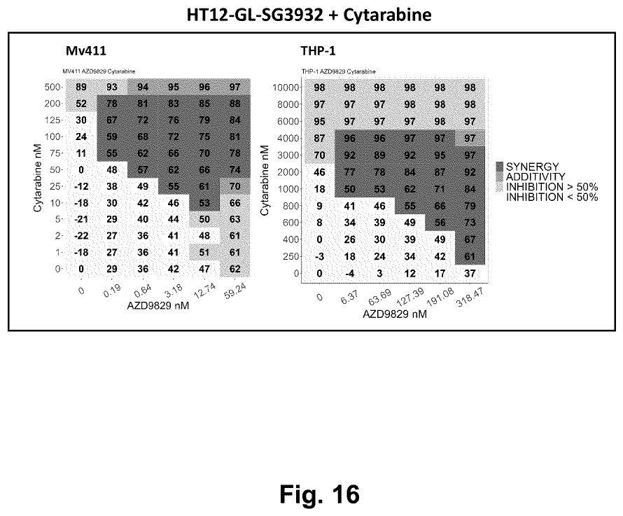

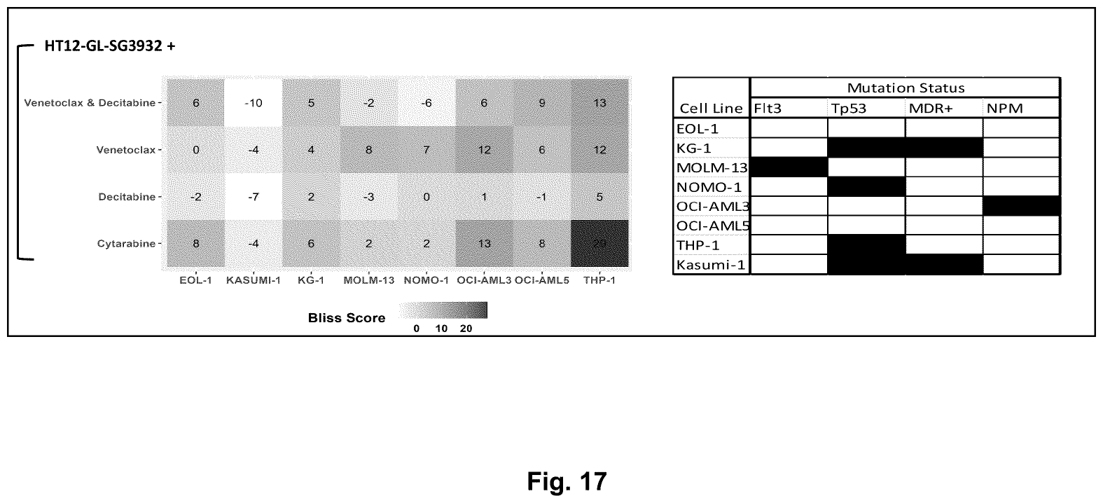

After the 6 day culture, cell viability data shows cytotoxicity synergy between HT12-GL-SG3932 and VEN (Group 5) with Bliss scores>5 in 5 of the 7 cell lines tested at doses ranging from 10 nM to 1 μM VEN and 0.1 nM-0.3 μM HT12-GL-SG3932 in THP-1 cells (FIGS. 15A and 17). HT12-GL-SG3932 and HMA (Group 6) showed cytoxic synergy with Bliss scores>5 in 1 of the 7 cell lines tested at doses ranging from 100 nM to 1 μM HMA and 0.1 nM-0.3 μM HT12-GL-SG3932 in THP-1 cells (FIGS. 15B and 17). The triple combination of HT12-GL-SG3932 with SoC (VEN and HMA) (Group 8) showed synergy with Bliss scores>5 in 5 of the 7 cell lines at doses ranging from 10 nM to 1 μM VEN, 10 nM to 0.5 μM HMA, and 0.1 nM-0.3 μM HT12-GL-SG3932 in THP-1 cells (FIGS. 15C and 17). HT12-GL-SG3932 and cytarabine (Group 7) showed cytoxic synergy with Bliss scores>5 in 5 of the 7 cell lines tested at doses ranging from 50 nM to 0.5 μM cytarabine and 0.1 nM-0.3 μM HT12-GL-SG3932 in THP-1 cells (FIGS. 16 and 17). Interestingly, all of treatment groups showed synergy in THP-1 cells, but none of the treatment groups demonstrated synergy in KASUMI-1 cells (FIG. 17).

These results demonstrate that treatment with HT12-GL-SG3932 synergizes with VEN, HMA, cytarabine or SoC (VEN and HMA) treatment in AML cell lines with diverse molecular alterations. As such, these preclinical findings suggest the potential of using combination therapy of HT12-GL-SG3932 and SoC (VEN and AZA) or cytarabine in the clinic to improve long-term outcomes for AML patients.

### Example 20—HT12-GL-SG3932 Combination Therapy with Standard of Care (HMA and Venetoclax) in AML Patient Samples

The effects of HT12-GL-SG3932 ADC (AZD9829) combination therapy with the standard of care (SoC) combination of DNA hypomethylating agent (HMA) and BCL2 inhibitor Venetoclax (VEN), was tested in primary AML patient samples ex vivo.

Primary AML patient sample cells from 11 patients were thawed and seeded at 0.5×10⁶ cells/ml in RPMI1640 medium with 10% of heat-inactivated FCS and a cytokine cocktail composed of FLT3-L (50 ng/ml), IL3(10 ng/ml), IL6 (10 ng/ml) and SCF (50 ng/ml) to support the cell survival. Cells were incubated at 37° C. in 5% CO2 atmosphere for 4 days (D4) and 7 days (D7). Twelve hours after thawing, “at Day 0 (D0)”, cells were treated with HT12-GL-SG3932 (125-500 nM) as a single agent or in combination with VEN (0.1 uM) or SoC (VEN and HMA (Azacitidine, 0.9 uM)). Cells were also treated with DMSO or NIP228-ADC (NIP228-SG3932) (125-500 nM) to serve as controls. Cytotoxic effects induced by treatments were assessed by flow cytometry at Day 4 and Day 7 post-treatment. The cytotoxic effect for all treatments was measured based on changes in the absolute number of blasts reported as mean % blast reduction (BR).

For flow cytometry, cells were washed and incubated with a viability marker for 15 minutes at 4° C. After a washing step, cells were incubated with Fc Blocker (BD) for 10 minutes as blocking step. Then, cell-surface staining was performed according to supplier's instructions and cells were incubated for 20 minutes at 4° C. Cells were then fixed and permeabilized using the Fixation/Permeabilization Solution Kit from BD. From this step, all washes were performed using the Perm/Wash solution. Nuclear membranes of cells were permeabilized using Permeabilization Buffer Plus from BD. To finish, intracellular staining was performed according to supplier's instructions and cells were incubated for 20 minutes at 4° C. Data were analyzed using Cytoflex cytometer (Beckman Coulter, Brea, CA, USA). The Gating Strategy is as described: First, contaminating events and dead cells were removed on histogram using live/dead viability marker. Single cells were plotted on FSC-A versus FSC-H to remove doublets. Blast and lymphocytes cells were identified by CD45/SSC gating: CD45 expression was moderate/dim for blasts and bright for lymphocytes. Blasts were characterized using CD33, CD34, CD38, CD117 and CD123 cell surface markers.

To assess the cytotoxic effect of molecules after 4 days and 7 days of treatment on the absolute number of blasts, for each condition, the relative number of cells and viability was measured using Trypan blue exclusion assay. Then, we analyzed the percentage of viable blasts for each condition using flow cytometry. Based on these percentages and Trypan blue exclusion assay, absolute cell numbers of each individual subpopulation were calculated using the formula: The relative number of cells of interest=total viable cell count*% of cells of interest.

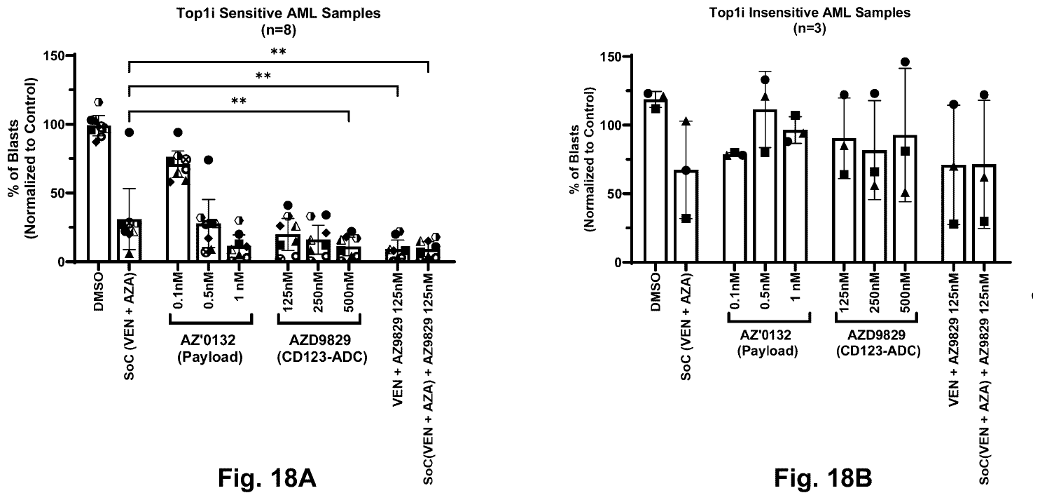

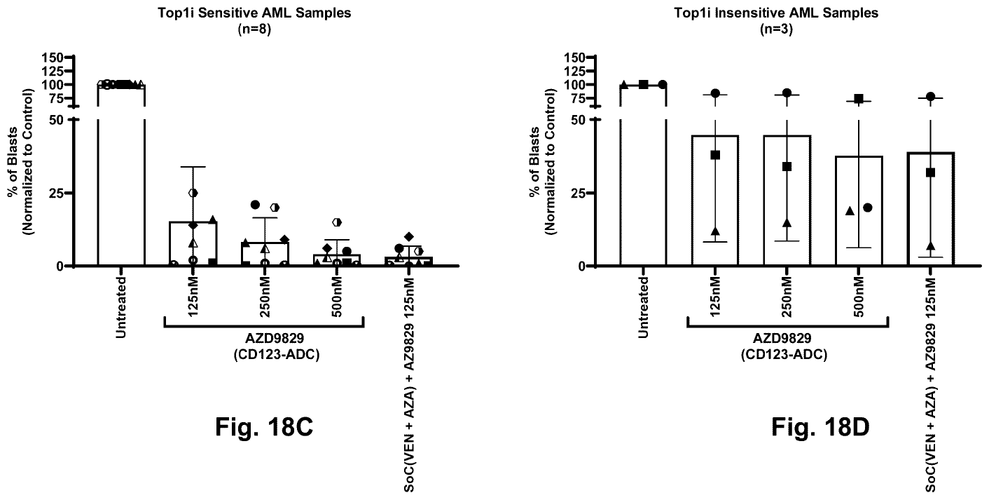

Eight of the eleven AML patient samples (i.e., Top1i sensitive samples, FIG. 18A (4 days) and 18C (7 days)) demonstrated significant blast reduction (BR) with 500 nM HT12-GL-SG3932 (89% BR) or with 1 nM Top1i payload alone (89% BR) compared to SoC (69% BR) The addition of 125 nM HT12-GL-SG3932 to SoC improved cell killing over SoC (91% vs. 69% BR, respectively). Although statistically insignificant, a greater percent blast reduction was observed with triple combination therapy compared to 125 nM HT12-GL-SG3932 as a single agent (91% vs. 80% BR, respectively) (FIG. 18A). Three patient samples did not respond to HT12-GL-SG3932 or Top1i payload (i.e., Top1i insensitive samples, FIGS. 18B (4 days) and 18D (7 days)).

These ex vivo results using primary AML patient samples demonstrate that treatment with HT12-GL-SG3932 as a single agent, and in combination with SoC, is more effective than SoC in AML patient samples with diverse molecular alterations. Therefore, these preclinical findings provide support to further investigate combination therapy of HT12-GL-SG3932 and SoC (VEN and AZA) in the clinic to improve long-term AML patient outcomes.

### Example 21—HT12-GL-SG3932 Combination Therapy with Standard of Care (HMA and Venetoclax) in AML PDX Model

The effects of HT12-GL-SG3932 ADC (AZD9829) combination therapy with the standard of care (SoC) combination of DNA hypomethylating agent (HMA) and BCL2 inhibitor Venetoclax (VEN), was tested in primary AML patient sample derived xenotransplant models (AML PDX models) in vivo.

Two AML PDX models (DFAM-68555 and DFAL-49600) were used to test the potential combo benefit of HT12-GL-SG3932 and SoC (VEN+AZA). AML DFAM-68555 is characterized by a Flt3 mutation and AML DFAM-49600 is characterized by a Tp53 mutation. AML PDX cells were thawed and counted, and one million (10⁶) cells were intravenously implanted to NSG mice. Peripheral blood engraftment (% of huCD45) was sporadically checked by flow cytometric analysis from the whole cohort or a subgroup of mice in order to determine the dosing stage. Body weight was used to randomize the mice using the Matched Distribution method built into the StudyLog software. Venetoclax was given orally at 50 mg/kg or 100 mg/kg daily for 14 days. Azacitidine was given intraperitoneally at 1.25 mg/kg or 2.5 mg/kg daily for 5 days. HT12-GL-SG3932 was given intravenously at 2 mg/kg or 4 mg/kg weekly for 2 weeks. Human IgG was given intraperitoneally at 100 mg/kg 24 hrs prior to HT12-GL-SG3932 treatment.

Mice were closely monitored for clinical signs of AML, body weight loss and hind limb paralysis. Moribund mice were euthanized promptly, and the survival days were recorded.

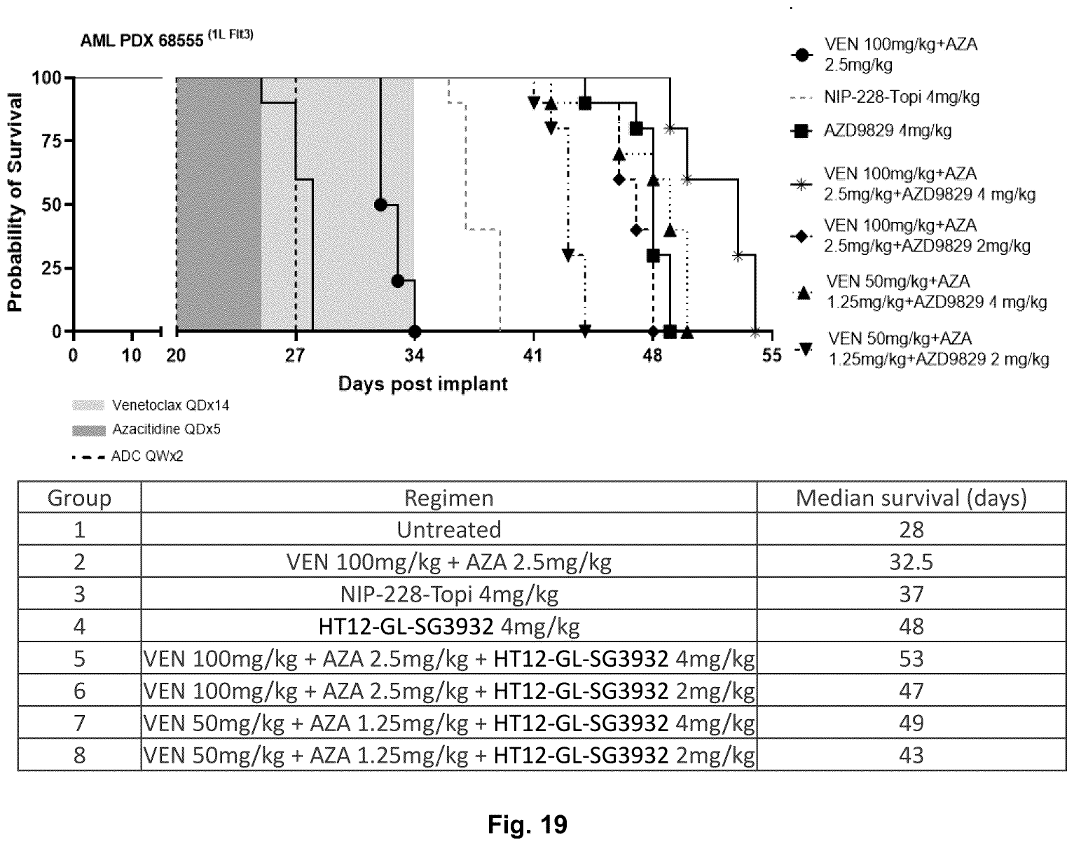

In a SoC-insensitive AML PDX (DFAM-68555) model, HT12-GL-SG3932 as a single agent significantly prolonged the median survival (48 days) compared to the untreated mice (28 days) and SoC-treated mice (32.5 days) (FIG. 19). The triple combination of HT12-GL-SG3932 with the SoC (Ven and AZA) further prolonged the median survival to 53 days (FIG. 19). Lower dose combination groups (Groups 6-8) had inferior or comparable survival benefit to HT12-GL-SG3932 monotherapy (FIG. 19).

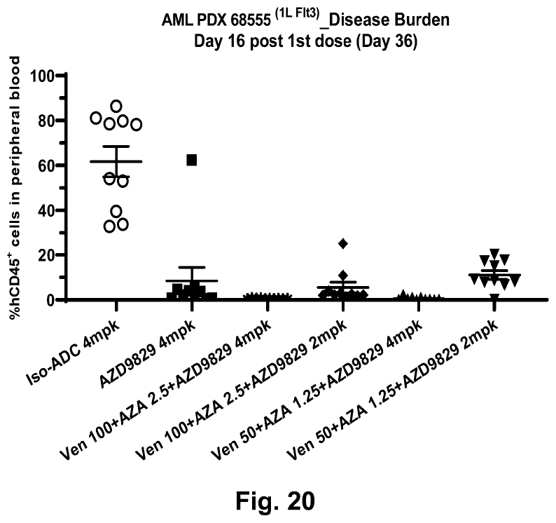

On Day 36 post-implant, peripheral blood were collected from all the remaining AML DFAM-68555 PDX mice. Blood samples were processed and stained for flow cytometric analysis to measure the percentage of human CD45%/live-dead. Both HT12-GL-SG3932 monotherapy group and all the combination groups significantly decreased the AML disease burden (human CD45%) in the blood (FIG. 20).

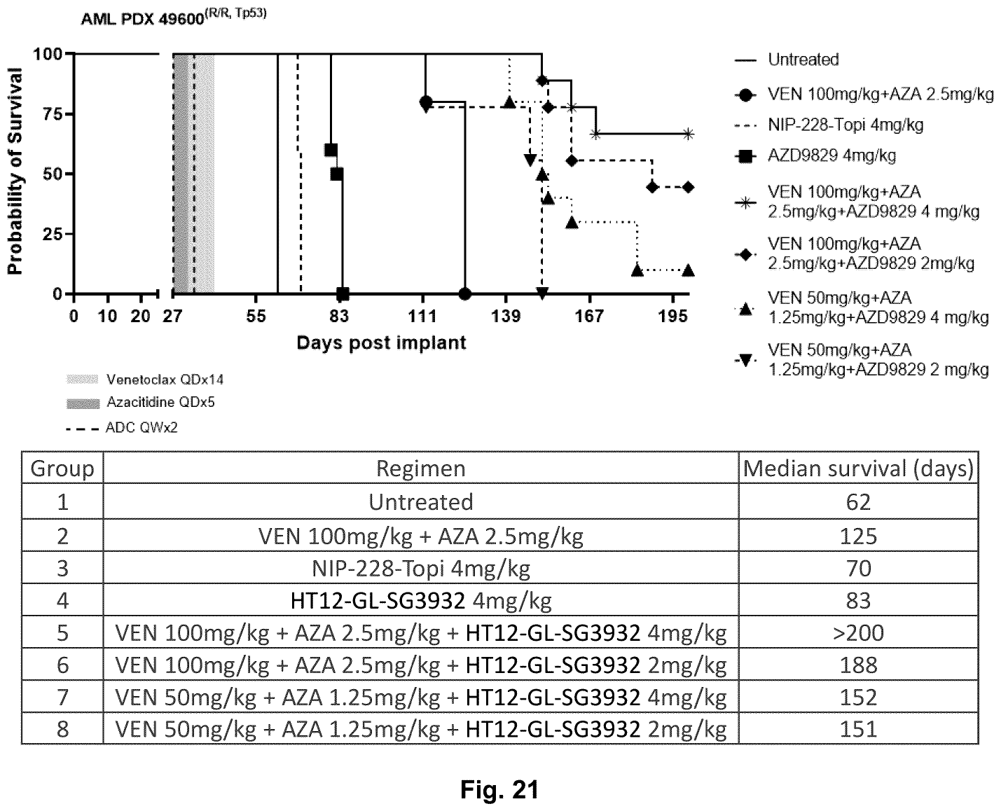

In a SoC-responsive AML PX (DFAL-49600) model, SoC prolonged the median survival to 125 days compared to the untreated mice (62 days) (FIG. 21). HT12-GL-SG3932 as a single agent also significantly prolonged the median survival (83 days) (FIG. 21). The triple combination was remarkably superior (>200 days) to both HT12-GL-SG3932 monotherapy and SoC (FIG. 21). All lower dose combination groups (Groups 6-8) had significantly prolonged median survival compared to either SoC or HT12-GL-SG3932 monotherapy (FIG. 21).

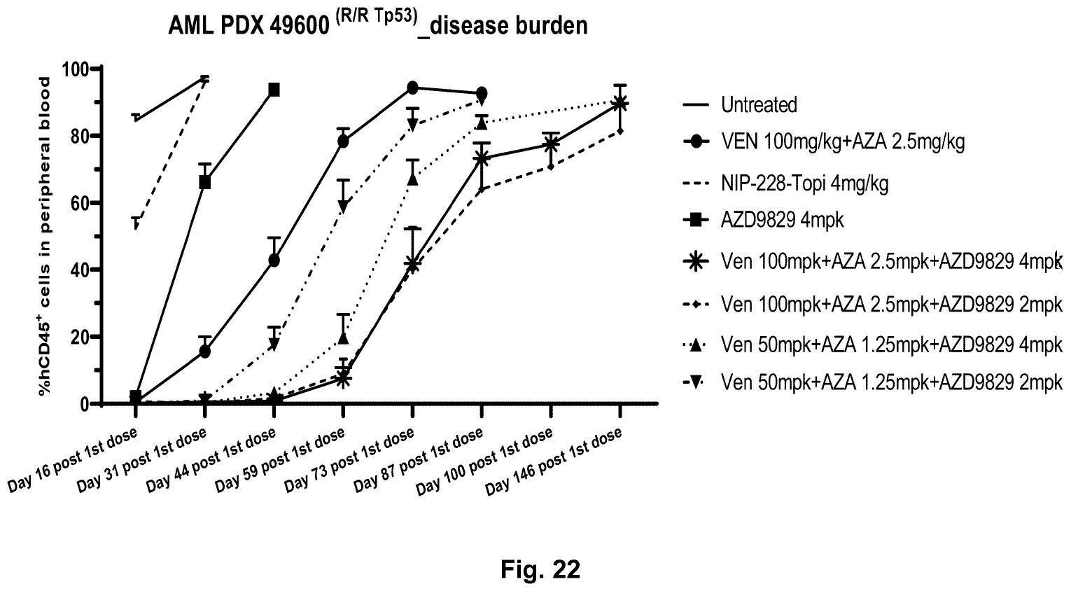

On Day 42, 57, 70, 85, 99, 113, 126, and 172 post-implant, peripheral blood were collected from all the remaining AML DFAL-49600 PDX mice. Blood samples were processed and stained for flow cytometric analysis to measure the percentage of human CD45%/live-dead. HT12-GL-SG3932 monotherapy group, SoC, and all the combination groups significantly decreased the AML disease burden (human CD45%) in the blood at the early time-points (FIG. 22).

These in vivo AML PDX model results demonstrate that treatment with HT12-GL-SG3932 as a single agent is effective in AML PDX models established with primary AML patient samples with a variety of molecular alternations, however combining HT12-GL-SG3932 with standard of care (SoC) prolongs the survival benefit. As such, these preclinical findings offer justification for exploring combination therapy of HT12-GL-SG3932 and SoC (VEN and AZA) in the clinic to improve long-term AML patient outcomes.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. An antibody that specifically binds CD123 comprising an antigen binding domain that comprises:
(i) a variable heavy chain region (VH) comprising a VH complementarity determining region (CDR) 1 comprising an amino acid sequence set forth in SEQ ID NO: 1, a VH-CDR2 comprising an amino acid sequence set forth in SEQ ID NO: 2, and a VH-CDR3 comprising an amino acid sequence set forth in SEQ ID NO: 3; and
(ii) a variable light chain region (VL) comprising a VL-CDR1 comprising an amino acid sequence set forth in SEQ ID NO: 4, a VL-CDR2 comprising an amino acid sequence set forth in SEQ ID NO: 5, and a VL-CDR3 comprising an amino acid sequence set forth in SEQ ID NO: 6.

2. (canceled)

3. (canceled)

4. (canceled)

5. (canceled)

6. The antibody of claim 1, wherein the antigen binding domain comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs:7.

7. (canceled)

8. The antibody of claim 1, wherein the antigen binding domain comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NO: 8.

9. (canceled)

10. (canceled)

11. The antibody of claim 1, wherein the antigen binding domain comprises:
(a) a VH comprising an amino acid sequence set forth in SEQ ID NO: 7 and a VL comprising an amino acid sequence set forth in SEQ ID NO: 8.

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A polynucleotide encoding the antibody of claim 11.

38. A vector comprising the polynucleotide of claim 37 operably linked to a promoter.

39. A cell comprising the polynucleotide of claim 37.

40. A conjugate of formula I:
A-(D^{L})ₚ (I)
or a pharmaceutically acceptable salt or solvate thereof, wherein A is an antibody of claim 1, D^{L} is a Drug Linker unit that is of formula II: 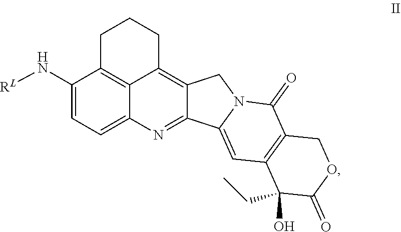
wherein R^{L} is of formula IIa: 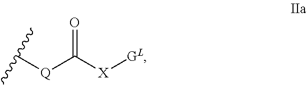
wherein
Q is: 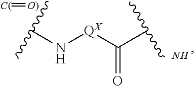
where Q^{X} is such that Q is an amino acid residue, a dipeptide residue, a tripeptide residue or a tetrapeptide residue, wherein the superscripted labels ^{C(═O)} and ^{NH} indicate the group to which the atoms are bound;
X is: 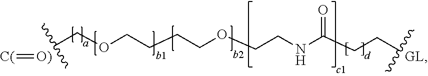
wherein a=0 to 5, b1=0 to 16, b2=0 to 16, c1=0 or 1, d=0 to 5; and GL is a linker for connecting to the antibody or antigen binding fragment thereof; and
wherein p is an integer of from 1 to 20.

41. The conjugate of claim 42, wherein Q is:
(a) an amino acid residue selected from: Phe, Lys, Val, Ala, Cit, Leu, Ile, Arg, and Trp; or
(b) a dipeptide residue selected from:
NH-Phe-Lys-C═O,
NH-Val-Ala-C═O,
NH-Val-Lys-C═O,
NH-Ala-Lys-C═O,
NH-Val-Cit-C═O,
NH-Phe-Cit-C═O,
NH-Leu-Cit-C═O,
NH-Ile-Cit-C═O,
NH-Phe-Arg-C═O,
NH-Trp-Cit-C═O, and
NH-Gly-Val-C═O; or
(c) a tripeptide residue selected from:
NH-Glu-Val-Ala-C═O,
NH-Glu-Val-Cit-C═O,
NH-αGlu-Val-Ala-C═O, and
NH-αGlu-Val-Cit-C═O; or
(d) a tetrapeptide residue selected from:
NH-Gly-Gly-Phe-Gly-C═O; and
NH-Gly-Phe-Gly-Gly-C═O, and
wherein NH represents the N-terminus, and C═O represents the C-terminus of the residue.

42. The conjugate of claim 40, wherein a is:
(a) 0 to 3; or
(b) 0 or 1; or
(c) 0;
wherein b1 is
(a) 0 to 8; or
(b) 0; or
(c) 2; or
(d) 3; or
(e) 4; or
(f) 5; or
(g) 8,
wherein b2 is
(a) 0 to 8; or
(b) 0; or
(c) 2; or
(d) 3; or
(e) 4; or
(f) 5; or
wherein c1 is
(a) 0; or
(b) 1; or
(c) 2; and
wherein d is
(a) 0 to 3; or
(b) 1 or 2; or
(c) 2; or
(d) 0.

43. (canceled)

44. (canceled)

45. (canceled)

46. (canceled)

47. The conjugate of claim 41, wherein:
(a) a is 0, b1 is 0, c1 is 1, d is 2, and b2 is 0, 2, 3, 4, 5 or 8; or
(b) a is 1, b2 is 0, c1 is 0, d is 0, and b1 is 0, 2, 3, 4, 5 or 8; or
(c) a is 0, b1 is 0, c1 is 0, d is 1, and b2 is 0, 2, 3, 4, 5 or 8; or
(d) b1 is 0, b2 is 0, c1 is 0, one of a and d is 0, and the other of a and d is 1 or 5; or
(e) a is 1, b2 is 0, c1 is 0, d is 2, and b1 is 0, 2, 3, 4, 5 or 8; or
(f) a is 0, b1 is 0, b2 is 8, c1 is 1, and d is 0.

48. The conjugate of claim 47, wherein GL is

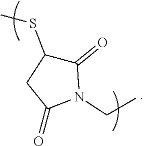

49. The conjugate of claim 48, wherein Q is NH-Val-Ala-C═O; wherein a is 1 or 0; wherein b1 is 0; wherein b2 is 8; wherein c1 is 1; and wherein d is 1 or 0.

50. (canceled)

51. (canceled)

52. (canceled)

53. (canceled)

54. (canceled)

55. The conjugate of claim 49, wherein R^{L} is:

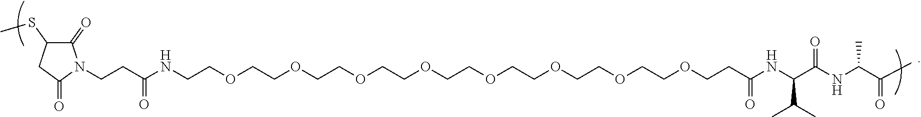

56. The conjugate of claim 55, wherein the antibody is an IgG antibody.

57. The conjugate of claim 55, wherein at least one D^{L} unit is bound to a hinge region of the IgG antibody.

58. The conjugate of claim 57, wherein 1 to 3 D^{L} units are bound to a heavy chain of the IgG antibody.

59. The conjugate of claim 58, wherein one D^{L} unit is bound to a light chain of the IgG antibody.

60. The conjugate of claim 59, wherein 1 to 3 D^{L} units are bound to each heavy chain of the IgG antibody and one D^{L} unit is bound to each light chain of the IgG antibody.

61. The conjugate of claim 60, wherein formula II is a single enantiomer or in an enantiomerically enriched form.

62. The conjugate of claim 61, wherein p is an integer of from 1 to about 10.

63. The conjugate of claim 62, wherein p is 8.

64. A conjugate comprising the antibody of claim 1, wherein the antibody or antigen binding fragment thereof is conjugated to a Drug Linker unit of:

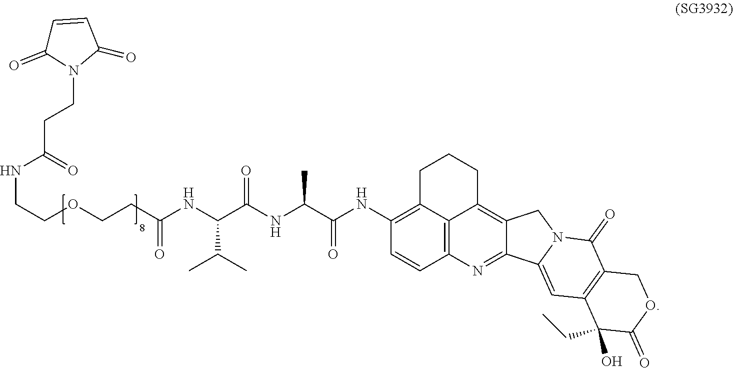

65. A mixture of conjugates comprising a conjugate of claim 64, wherein an average p in the mixture is from about 1 to about 10.

66. (canceled)

67. (canceled)

68. (canceled)

69. (canceled)

70. A method of treating a proliferative disease, the method comprising administering to a subject in need thereof the conjugate of claim 64.

71. The method of claim 70, wherein the proliferative disease is cancer.

72. The method of claim 71, wherein the cancer is a hematologic cancer.

73. The method of claim 72, wherein the hematologic cancer is a leukemia or lymphoma.

74. The method of any one of claim 71, wherein the cancer is acute myeloid leukemia, acute lymphoid leukemia, myelodysplastic syndrome, refractory anemia with excess blasts, DLBCL non-Hodgkin lymphoma, marginal zone non-Hodgkin lymphoma, mantle zone non-Hodgkin lymphoma, follicular non-Hodgkin lymphoma, Hodgkin lymphoma, or minimal residual disease.

75. (canceled)

76. (canceled)

77. (canceled)

78. (canceled)

79. An antibody-drug conjugate (ADC) comprising:
(i) an antibody which binds to CD123 comprising: a HCDR1 comprising the amino acid sequence as set forth in SEQ ID NO: 1; a HCDR2 comprising the amino acid sequence as set forth in SEQ ID NO: 2; a HCDR3 comprising the amino acid sequence as set forth in SEQ ID NO: 3; and a LCDR1 comprising the amino acid sequence as set forth in SEQ ID NO: 4; a LCDR2 comprising the amino acid sequence as set forth in SEQ ID NO: 5; and a LCDR3 comprising the amino acid sequence as set forth in SEQ ID NO: 6;
(ii) wherein one or more cysteine residues of the antibody or antigen binding fragment are conjugated to: 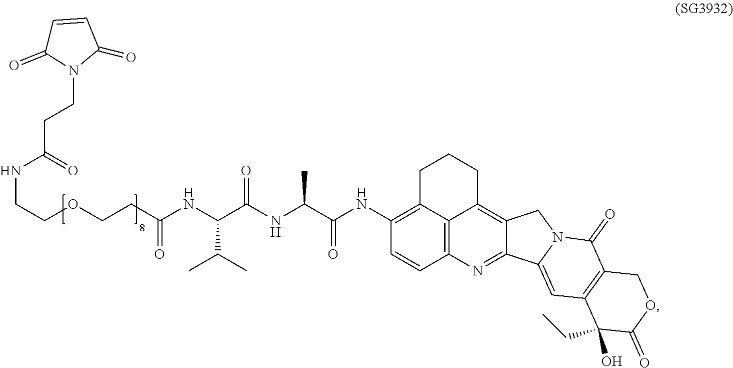
and wherein the ADC has a drug to antibody ratio (DAR) of about 8.

80. The ADC of claim 79, wherein the antibody comprises a variable heavy (VH) chain comprising the amino acid sequence as set forth in SEQ ID NO: 7 and a variable light (VL) chain comprising the amino acid sequence as set forth in SEQ ID NO: 8.

81. The ADC of claim 80, comprising a heavy chain (HC) comprising the amino acid sequence of SEQ ID NO: 116, and a light chain (LC) comprising the amino acid sequence of SEQ ID NO: 117.

82. (canceled)

83. A method of treating a cancer comprising administering the ADC of claim 81 to a patient.

84. The method of claim 83, wherein the cancer is acute myeloid leukemia or myelodysplastic syndrome.

85. (canceled)

86. (canceled)

87. The method of claim 83, further comprising administering venetoclax and/or a hypomethylating agent, wherein the hypomethylating agent is 5-azacytidine or decitabine.
